(19) United States
(12) Patent Application Publication
(10) Pub. No.: US 2022/0073525 A1
(43) Pub. Date: Mar. 10, 2022

(54) COMPOSITIONS FOR INHIBITING UBIQUITIN SPECIFIC PROTEASE 1

(71) Applicant: FORMA Therapeutics, Inc., Watertown, MA (US)

(72) Inventors: Alexandre Joseph Buckmelter, Acton, MA (US); Justin Andrew Caravella, Cambridge, MA (US); Jian Lin, Acton, MA (US); Edward L. Fritzen, Niantic, CT (US)

(73) Assignee: FORMA Therapeutics, Inc., Watertown, MA (US)

(21) Appl. No.: 17/417,805

(22) PCT Filed: Dec. 27, 2019
(86) PCT No.: PCT/US19/68648

§ 371 Date: Jun. 24, 2021

(51) Int. Cl. C07D 487/04

(52) U.S. Cl. CPC C07D 487/04

(57) ABSTRACT

This disclosure relates to modulating ubiquitin specific protease 1 (USP1) and provides novel chemical compounds useful as inhibitors of USP1, as well as various uses of these compounds. USP1 inhibiting compounds are useful in the treatment of diseases and disorders associated with USP1, such as cancer.

### RELATED APPLICATIONS

The present application claims priority to U.S. Provisional Application No. 62/785,733, filed Dec. 28, 2018, the entire contents of which are hereby incorporated by reference.

### TECHNICAL FIELD

This disclosure relates to novel chemical compositions for inhibiting ubiquitin specific protease 1.

### BACKGROUND

Ubiquitination is a reversible process which involves a family of deubiquitinating enzymes (DUBs) that regulate a variety of cellular processes by deconjugating ubiquitin from the substrate. DUBs are encoded by approximately 100 human genes and are classified into six families, with the largest family being the ubiquitin-specific proteases (USPs). Ubiquitin-specific protease 1 (USP1) is a cysteine isopeptidase of the USP subfamily of DUBs. USP1 deubiquitinates a variety of cellular targets involved in different processes related to cancer.

In particular, USP1 is known to promote tumor stem cell maintenance and radioresistance in glioblastoma via stabilization of ID1 and CHEK1. Further, USP1 is known to play a role in regulating cell proliferation and differentiation through deubiquitinating and stabilizing inhibitors of DNA binding (IDs) that antagonize basic helix-loop-helix (bHLH) transcription factors. shRNA knockdown of USP1 in U2OS cells induces cell cycle arrest via ID proteins and shRNA knockdown of USP1 in 143B human osteosarcoma xenografts inhibits tumor growth result. Inhibition of USP1, therefore, is useful for treating for diseases and disorders associated with modulation of USP1.

### SUMMARY

The present disclosure provides technologies useful for inhibiting USP1. In some embodiments, provided technologies are useful for, among other things, treating certain diseases and disorders associated with USP1. In some embodiments, provided technologies are useful for, among other things, treating cancer.

In some embodiments, the present disclosure provides compounds of Formula I:

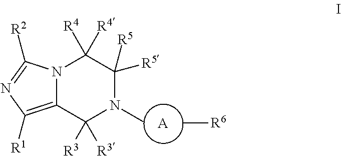

or a pharmaceutically acceptable salt thereof, wherein:
Ring A is —C₃-C₁₂ cycloalkyl, 3- to 14-membered heterocyclyl having 1 to 4 heteroatoms selected from oxygen, nitrogen, and sulfur, —C₆-C₁₀ aryl, or 5- to 14-membered heteroaryl having 1 to 4 heteroatoms selected from oxygen, nitrogen, and sulfur,
   wherein each cycloalkyl, heterocyclyl, aryl, or heteroaryl of Ring A is optionally substituted with one or more substituents selected from the group consisting of halogen, —C₁-C₆ alkyl, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, and —S(O)₂NR₂;
R¹ is —C₆-C₁₀ aryl or 5- to 14-membered heteroaryl having 1 to 4 heteroatoms selected from oxygen, nitrogen, and sulfur,
   wherein each aryl or heteoraryl of R¹ is optionally substituted with one or more R^{1a};
each R^{1a} is independently halogen, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, —C₁-C₆ alkyl, —C₂-C₆ alkenyl, —C₂-C₆ alkynyl, —C₃-C₁₂ cycloalkyl, —C₄-C₁₂ cycloalkenyl, 3- to 14-membered heterocyclyl having 1 to 4 heteroatoms selected from oxygen, nitrogen, or sulfur, —C₆-C₁₀ aryl, or 5- to 14-membered heteroaryl having 1 to 4 heteroatoms selected from oxygen, nitrogen, or sulfur;
R² is —H, halogen, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, —C₁-C₆ alkyl, —C₂-C₆ alkenyl, —C₂-C₆ alkynyl, —C₃-C₁₂ cycloalkyl, —C₄-C₁₂ cycloalkenyl, 3- to 14-membered heterocyclyl having 1 to 4 heteroatoms selected from oxygen, nitrogen, or sulfur, —C₆-C₁₀ aryl, or 5- to 14-membered heteroaryl having 1 to 4 heteroatoms selected from oxygen, nitrogen, or sulfur,
   wherein each alkyl, alkenyl, alkynyl, cycloalkyl, cycloalkenyl, heterocyclyl, aryl, or heteroaryl of R² is optionally substituted with one or more halogen, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, —C₁-C₆ alkyl, —C₃-C₁₂ cycloalkyl, 3- to 14-membered heterocyclyl having 1 to 4 heteroatoms selected from oxygen, nitrogen, or sulfur, —C₆-C₁₀ aryl, or 5- to 14-membered heteroaryl having 1 to 4 heteroatoms selected from oxygen, nitrogen, or sulfur;
R³, R^{3′}, R⁴, R^{4′}, R⁵, and R^{5′} are each independently selected from the group consisting of —H, —C₁-C₆ alkyl, —C₂-C₆ alkenyl, —C₂-C₆ alkynyl, and —C₃-C₇ cycloalkyl; or each of (R³ and R^{3′}), or (R⁴ and R^{4′}), or (R⁵ and R^{5′}) can combine with the atom to which they are attached to form a —C₃-C₁₂ cycloalkyl ring or 3- to 14-membered heterocyclyl ring having 1 to 4 heteroatoms selected from oxygen, nitrogen, or sulfur;
R⁶ is —H, halogen, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, —C₁-C₆ alkyl, —C₂-C₆ alkenyl, —C₂-C₆ alkynyl, —C₃-C₁₂ cycloalkyl, —C₄-C₁₂ cycloalkenyl, 3- to 14-membered heterocyclyl having 1 to 4 heteroatoms selected from oxygen, nitrogen, or sulfur, —C₆-C₁₀ aryl, or 5- to 14-membered heteroaryl having 1 to 4 heteroatoms selected from oxygen, nitrogen, or sulfur,
   wherein each alkyl, alkenyl, alkynyl, cycloalkyl, cycloalkenyl, heterocyclyl, aryl or heteroaryl of R⁶ is optionally substituted with one or more R^{6a};
each R^{6a} is independently selected from the group consisting of halogen, oxo, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, —C₁-C₆ alkyl, —C₂-C₆ alkenyl, —C₂-C₆ alkynyl, —C₃-C₁₂ cycloalkyl, —C₄-C₁₂ cycloalkenyl, 3- to 14-membered heterocyclyl having 1 to 4 heteroatoms selected from oxygen, nitrogen, or sulfur, —C₆-C₁₀ aryl, and 5- to 14-membered heteroaryl having 1 to 4 heteroatoms selected from oxygen, nitrogen, or sulfur,
   wherein each alkyl, alkenyl, alkynyl, cycloalkyl, cycloalkenyl, heterocyclyl, aryl, or heteroaryl of R^{6a} is optionally substituted with one or more halogen, —C₁-C₆ alkyl, or —OR;
each R is independently selected from the group consisting of —H, —C₁-C₆ alkyl, —C₀-C₆ alkylene-C₆-C₁₀ aryl, —C₃-C₁₂ cycloalkyl, 3- to 14-membered heterocyclyl having 1 to 4 heteroatoms selected from oxygen, nitrogen, or sulfur, or 5- to 14-membered heteroaryl having 1 to 4 heteroatoms selected from oxygen, nitrogen, or sulfur,
   wherein each alkyl, aryl, cycloalkyl, heterocyclyl, or heteroaryl of R is optionally substituted with one or more R^{a};
or two R groups can combine with the atom to which they are attached to form a 5- to 6-membered heterocyclyl having 1 to 4 heteroatoms selected from oxygen, nitrogen, or sulfur, optionally substituted with —C₁-C₆ alkyl;
each R^{a} is independently halogen, —O(C₁-C₆ alkyl), —NH(C₁-C₆ alkyl), —N(C₁-C₆ alkyl)₂, —C₃-C₆ cycloalkyl, 5- to 6-membered heterocyclyl having 1 to 4 heteroatoms selected from oxygen, nitrogen, or sulfur, or 5- to 6-membered heteroaryl having 1 to 4 heteroatoms selected from oxygen, nitrogen, or sulfur,
   wherein each cycloalkyl, heterocyclyl, or heteroaryl of R^{a} is optionally substituted with —C₁-C₆ alkyl or —C₁-C₆ alkyl substituted with one or more halogen; and
each R′ is independently selected from the group consisting of —C₁-C₆ alkyl, —C₂-C₆ alkenyl, —C₂-C₆ alkynyl, —C₃-C₁₂ cycloalkyl, —C₄-C₁₂ cycloalkenyl, 3- to 14-membered heterocyclyl having 1 to 4 heteroatoms selected from oxygen, nitrogen, or sulfur, —C₆-C₁₀ aryl, and 5- to 14-membered heteroaryl having 1 to 4 heteroatoms selected from oxygen, nitrogen, or sulfur.

In some embodiments, the present disclosure provides compounds of Formula I that are USP1 Inhibitors. In some embodiments, a USP1 Inhibitor is a compound of Formula I that has an IC₅₀ value of ≤1 μM in the Biochemical Assay of Example 1. In some embodiments, a USP1 Inhibitor is a compound of Formula I that has an IC₅₀ value of ≤10 μM in the Biochemical Assay of Example 1. In some embodiments, a USP1 Inhibitor is a compound of Formula I that has an IC₅₀ value of ≤25 μM in the Biochemical Assay of Example 1.

### DETAILED DESCRIPTION

### USP1 Inhibition and Associated Disorders

Ubiquitination is a reversible process which involves a family of deubiquitinating enzymes (DUBs) that regulate a variety of cellular processes by deconjugating ubiquitin from the substrate. DUBs and their substrate proteins are often deregulated in cancers, suggesting that targeting specific DUB family members will result in antitumor activity through enhancing the ubiquitination and subsequent degradation of oncogenic substrates and the activity of other key proteins involved in tumor growth, survival, differentiation and maintenance of the tumor microenvironment.

Ubiquitin-specific protease 1 (USP1) is a cysteine isopeptidase of the USP subfamily of DUBs. Full-length human USP1 is composed of 785-amino acids, including a catalytic triad composed of Cys90, His593 and Asp751. USP1 deubiquitinates a variety of cellular targets involved in different processes related to cancer, including, for example, PCNA (proliferating cell nuclear antigen) and FANCD2 (Fanconi anemia group complementation group D2), both of which are involved in DNA damage response (DDR) pathways. These DNA damage response (DDR) pathways are essential for repair of DNA damage induced by DNA cross-linking agents such as cisplatin, mitomycin C, diepoxybutane, ionizing radiation and ultraviolet radiation.

In vivo studies in mouse embryonic fibroblasts (MEFs) from USP1-deficient mice show increased levels of Ub-PCNA and Ub-FANCD2 in chromatin, demonstrate impaired FANCD2 foci assembly and are defective in homologous recombination repair. Disruption of the USP1 gene in chicken cells (DT40) has been shown to result in DNA crosslinker hypersensitivity. Moreover, depletion of USP1 in human cell lines by siRNA results in elevated Ub-PCNA levels with increased recruitment of DNA polymerases specialized for translesion synthesis.

In addition to regulating protein dynamics in DDR pathways, USP1 promotes tumor stem cell maintenance and radioresistance in glioblastoma via stabilization of ID1 and CHEK1 and plays a role in regulating proliferation and differentiation through deubiquitinating and stabilizing inhibitors of DNA binding (IDs) that antagonize basic helix-loop-helix (bHLH) transcription factors. shRNA knockdown of USP1 in U2OS cells induces cell cycle arrest via ID proteins and shRNA knockdown of USP1 in 143B human osteosarcoma xenografts inhibits tumor growth result.

Accordingly, the present disclosure provides certain compounds and/or compositions that act as USP1 inhibitor agents, and technologies related thereto.

### Compounds

In some embodiments, the present disclosure provides a compound of Formula I.

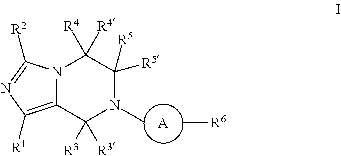

or a pharmaceutically acceptable salt thereof,
wherein Ring A, R¹, R², R³, R^{3′}, R⁴, R^{4′}, R⁵, R^{5′}, and R⁶ are as defined above and described in classes and subclasses herein, both singly and in combination.

In some embodiments, a compound of Formula I, or a pharmaceutically acceptable salt thereof, is provided wherein:
Ring A is —C₆-C₁₀ aryl or 5- to 14-membered heteroaryl having 1 to 4 heteroatoms selected from oxygen, nitrogen, and sulfur, wherein each aryl or heteroaryl of Ring A is optionally substituted with one or more substituents selected from the group consisting of halogen or —C₁-C₆ alkyl;
R¹ is —C₆-C₁₀ aryl or 5- to 14-membered heteroaryl having 1 to 4 heteroatoms selected from oxygen, nitrogen, and sulfur,
   wherein each aryl or heteoraryl of R¹ is substituted with 0, 1, 2, 3, or 4 R^{1a};
each R^{1a} is independently —OR or —C₁-C₆ alkyl;
R² is —H, —OR, —C₁-C₆ alkyl, or —C₃-C₁₂ cycloalkyl, wherein each alkyl of R² is optionally substituted with one or more halogen;
R³, R^{3′}, R⁴, R^{4′}, R⁵, and R^{5′} are each independently selected from the group consisting of —H or —C₁-C₆ alkyl;
R⁶ is —H, halogen, —OR, —NR₂, —NRC(O)R′, —CN, —C(O)NR₂, —C₁-C₆ alkyl, 3- to 14-membered heterocyclyl having 1 to 4 heteroatoms selected from oxygen, nitrogen, or sulfur, or 5- to 14-membered heteroaryl having 1 to 4 heteroatoms selected from oxygen, nitrogen, or sulfur,
   wherein each alkyl, heterocyclyl, or heteroaryl of R⁶ is substituted with 0, 1, 2, 3, or 4 R^{6a};
each R^{6a} is independently selected from the group consisting of halogen, oxo, —OR, —C₁-C₆ alkyl, —C₃-C₁₂ cycloalkyl, or 3- to 14-membered heterocyclyl having 1 to 4 heteroatoms selected from oxygen, nitrogen, or sulfur,
   wherein each alkyl, cycloalkyl, or heterocyclyl of R^{6a} is optionally substituted with one or more halogen, —C₁-C₆ alkyl, or —OR;
each R is independently selected from the group consisting of —H, —C₁-C₆ alkyl, —C₀-C₆ alkylene-C₆-C₁₀ aryl, or —C₃-C₁₂ cycloalkyl,
   wherein each alkyl, aryl, or cycloalkyl of R is substituted with 0, 1, 2, 3, or 4 R^{a};
or two R groups can combine with the atom to which they are attached to form a 5- to 6-membered heterocyclyl having 1 to 4 heteroatoms selected from oxygen, nitrogen, or sulfur, optionally substituted with —C₁-C₆ alkyl;
each R^{a} is independently halogen, —C₃-C₆ cycloalkyl, 5- to 6-membered heterocyclyl having 1 to 4 heteroatoms selected from oxygen, nitrogen, or sulfur, or 5- to 6-membered heteroaryl having 1 to 4 heteroatoms selected from oxygen, nitrogen, or sulfur,
   wherein each cycloalkyl, heterocyclyl, or heteroaryl of R^{a} is optionally substituted with —C₁-C₆ alkyl substituted with one or more halogen; and
each R′ is independently selected from the group consisting of —C₃-C₁₂ cycloalkyl.

In some embodiments, a compound of Formula I, or a pharmaceutically acceptable salt thereof, is provided wherein:
Ring A is phenyl, pyridyl, pyrazinyl, pyrimidinyl, or indolyl,
   wherein each phenyl of Ring A is optionally substituted with one or more substituents selected from the group consisting of fluoro and methyl;
R¹ is phenyl or pyridyl,
   wherein each phenyl or pyridyl of R¹ is substituted with 0, 1, 2, 3, or 4 R^{1a};
each R^{1a} is independently —OCH(CH₃)₂, —OCHF₂, —OCH₂CF₃, or isopropyl;
R² is —H, —OCH₃, methyl, or cyclopropyl,
   wherein each methyl of R² is optionally substituted with one or more fluoro;
R³, R^{3′}, R⁴, R^{4′}, R⁵, and R^{5′} are each independently selected from the group consisting of —H and methyl;
R⁶ is —H, fluoro, —OR,—NMe₂, —NHC(O)R′, —CN, —C(O)NR₂, methyl, a heterocyclyl selected from the group consisting of dihydroisoxazolyl, pyrrolidinyl, piperidinyl, oxazolidinyl, morpholinyl, and oxetanyl, or a heteroaryl selected from the group consisting of imidazolyl, pyrazolyl, oxazolyl, pyrimidinyl, tetrazolyl, triazolyl, and oxadiazolyl, wherein each heterocyclyl or heteroaryl of R⁶ is substituted with 0, 1, 2, 3, or 4 R^{6a};
each R^{6a} is independently selected from the group consisting of chloro, oxo, —OH, methyl, ethyl, isobutyl, cyclopropyl, azetidinyl, oxetanyl, or tetrahydropyranyl,
   wherein each methyl, ethyl, or azetidinyl of R^{6a} is optionally substituted with one or more fluoro, methyl, —OCH₃, or —OH;
each R is independently selected from the group consisting of —H, methyl, ethyl, isopropyl, —CH₂-phenyl, or cyclopropyl,
   wherein each methyl, ethyl, or phenyl of R is substituted with 0, 1, 2, 3, or 4 R^{a};
or two R groups can combine with the nitrogen to which they are attached to form a pyrrolidine, optionally substituted with methyl;
each R^{a} is independently fluoro, cyclopropyl, morpholinyl, or pyrazolyl,
   wherein each pyrazolyl of R^{a} is optionally substituted with —CF₃; and
each R′ is cyclopropyl.

In some embodiments, a compound of Formula IIa or IIb is provided:

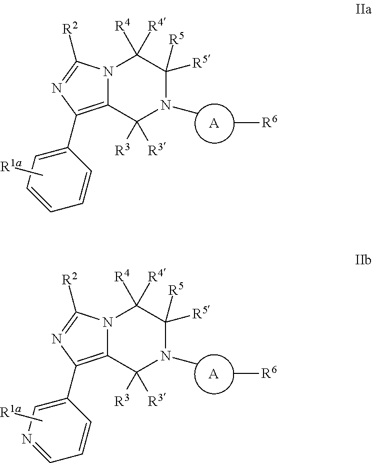

or a pharmaceutically acceptable salt thereof,
wherein Ring A, R^{1a}, R², R³, R^{3′}, R⁴, R^{4′}, R⁵, R^{5′}, and R⁶ are as defined above and described in classes and subclasses herein, both singly and in combination.

In some embodiments, a compound of Formula IIIa or IIIb is provided:

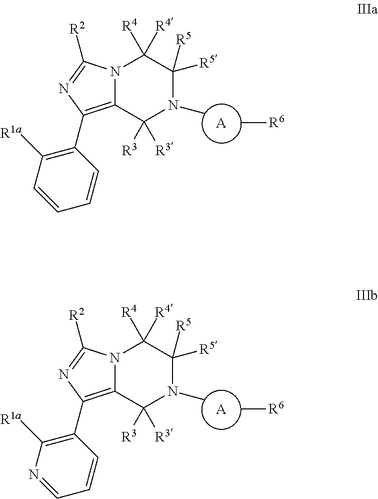

or a pharmaceutically acceptable salt thereof,
wherein Ring A, R^{1a}, R², R³, R^{3′}, R⁴, R^{4′}, R⁵, R^{5′}, and R⁶ are as defined above and described in classes and subclasses herein, both singly and in combination.

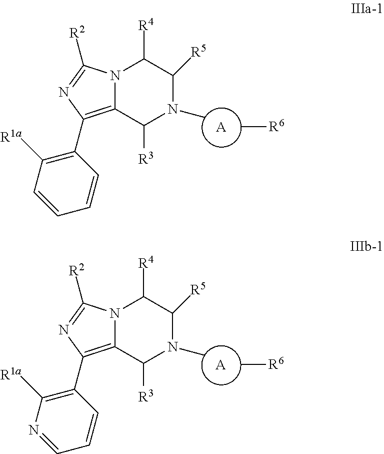

or a pharmaceutically acceptable salt thereof,
wherein Ring A, R^{1a}, R², R³, R⁴, R⁵, and R⁶ are as defined above and described in classes and subclasses herein, both singly and in combination.

In some embodiments, a compound of Formula IIIa-1 or IIIb-1, or a pharmaceutically acceptable salt thereof, is provided wherein:
Ring A is —C₆-C₁₀ aryl or 5- to 14-membered heteroaryl having 1 to 4 heteroatoms selected from oxygen, nitrogen, and sulfur,
   wherein each aryl or heteroaryl of Ring A is optionally substituted with one or more halogen;
each R^{1a} is independently —OR or —C₁-C₆ alkyl;
R² is —H, —OR, —C₁-C₆ alkyl, or —C₃-C₁₂ cycloalkyl,
   wherein each alkyl or cycloalkyl of R² is optionally substituted with one or more halogen;
R³, R⁴, and R⁵ are each independently selected from the group consisting of —H and —C₁-C₆ alkyl;
R⁶ is —OR, —NRC(O)R′, —C(O)NR₂, —C₁-C₆ alkyl, 3- to 14-membered heterocyclyl having 1 to 4 heteroatoms selected from oxygen, nitrogen, or sulfur, or 5- to 14-membered heteroaryl having 1 to 4 heteroatoms selected from oxygen, nitrogen, or sulfur,
   wherein each heterocyclyl or heteroaryl of R⁶ is substituted with 0, 1, 2, 3, or 4 R^{6a};
each R^{6a} is independently selected from the group consisting of halogen, oxo, —OR, —C₁-C₆ alkyl, —C₃-C₁₂ cycloalkyl, or 3- to 14-membered heterocyclyl having 1 to 4 heteroatoms selected from oxygen, nitrogen, or sulfur,
   wherein each alkyl, cycloalkyl, or heterocyclyl of R^{6a} is optionally substituted with one or more halogen, —C₁-C₆ alkyl, or —OR;
each R is independently selected from the group consisting of —H, —C₁-C₆ alkyl or —C₀-C₆ alkylene-C₆-C₁₀ aryl,
   wherein each alkyl or aryl of R is substituted with 0, 1, 2, 3, or 4 R^{a};
or two R groups can combine with the atom to which they are attached to form a 5- to 6-membered heterocyclyl having 1 to 4 heteroatoms selected from oxygen, nitrogen, or sulfur, optionally substituted with —C₁-C₆ alkyl;
each R^{a} is independently halogen, 5- to 6-membered heterocyclyl having 1 to 4 heteroatoms selected from oxygen, nitrogen, or sulfur, or 5- to 6-membered heteroaryl having 1 to 4 heteroatoms selected from oxygen, nitrogen, or sulfur,
   wherein each heterocyclyl or heteroaryl of R^{a} is optionally substituted with —C₁-C₆ alkyl substituted with one or more halogen; and
each R′ is independently —C₃-C₁₂ cycloalkyl.

In some embodiments, a compound of Formula IIIa-1 or IIIb-1, or a pharmaceutically acceptable salt thereof, is provided wherein:
Ring A is phenyl, pyridyl, pyrazinyl, or pyrimidinyl,
   wherein each phenyl of Ring A is optionally substituted with one or more fluoro;
R¹ is phenyl or pyridyl,
   wherein each phenyl or pyridyl of R¹ is substituted with 0, 1, 2, 3, or 4 R^{1a};
each R^{1a} is independently —OCH(CH₃)₂, —OCHF₂, —OCH₂CF₃, or isopropyl;
R² is —H, —OCH₃, methyl, or cyclopropyl,
   wherein each methyl of R² is optionally substituted with one or more fluoro;
R³, R⁴, and R⁵ are each independently selected from the group consisting of —H and methyl;
R⁶ is —OR, —NHC(O)R′, —C(O)NR₂, methyl, a heterocyclyl selected from the group consisting of dihydroisoxazolyl, pyrrolidinyl, and piperidinyl, or a heteroaryl selected from the group consisting of imidazolyl, pyrazolyl, oxazolyl, pyrimidinyl, tetrazolyl, triazolyl, and oxadiazolyl,
   wherein each heterocyclyl or heteroaryl of R⁶ is substituted with 0, 1, 2, 3, or 4 R^{6a}.
each R^{6a} is independently selected from the group consisting of chloro, oxo, —OH, methyl, ethyl, isobutyl, cyclopropyl, azetidinyl, oxetanyl, or tetrahydropyranyl,
   wherein each methyl, ethyl, or azetidinyl of R^{6a} is optionally substituted with one or more fluoro, methyl, —OMe, or —OH;
each R is independently selected from the group consisting of —H, methyl, ethyl, isopropyl, or —CH₂-phenyl,
   wherein each methyl, ethyl, or phenyl of R is substituted with 0, 1, 2, 3, or 4 R^{a};
or two R groups can combine with the nitrogen to which they are attached to form a pyrrolidine, optionally substituted with methyl;
each R^{a} is independently fluoro, morpholinyl, or pyrazolyl,
   wherein each pyrazolyl of R^{a} is optionally substituted with —CF₃; and
each R′ is cyclopropyl.

In some embodiments, a compound of Formula IIIa-2 or IIIb-2 is provided:

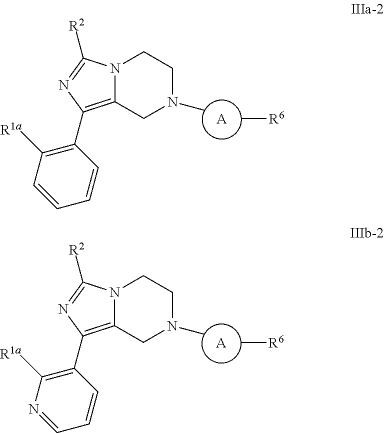

or a pharmaceutically acceptable salt thereof,
wherein Ring A, R^{1a}, R², and R⁶ are as defined above and described in classes and subclasses herein, both singly and in combination.

In some embodiments, a compound of Formula IVa or IVb is provided:

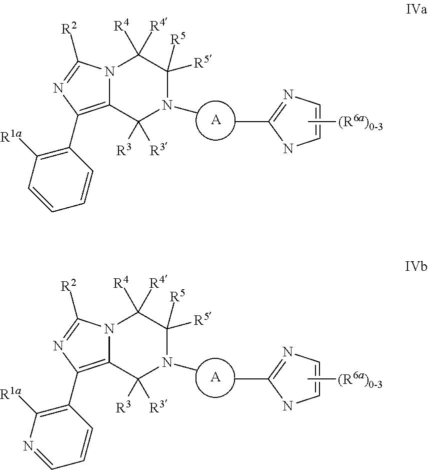

or a pharmaceutically acceptable salt thereof,
wherein Ring A, R^{1a}, R², R³, R³, R⁴, R^{4′}, R⁵, R^{5′}, and R^{6a} are as defined above and described in classes and subclasses herein, both singly and in combination.

In some embodiments, a compound of Formula IVa-1 or IVb-1 is provided:

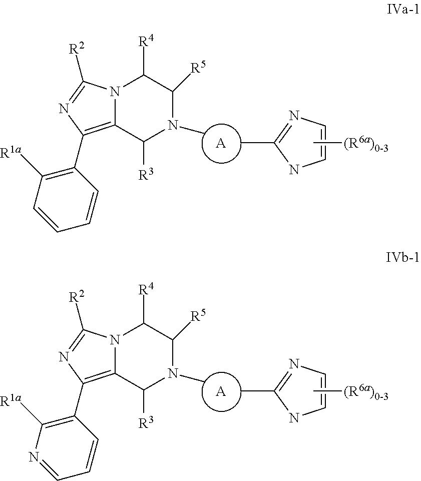

or a pharmaceutically acceptable salt thereof,
wherein Ring A, R^{1a}, R², R³, R⁴, R⁵, and R^{6a} are as defined above and described in classes and subclasses herein, both singly and in combination.

In some embodiments, a compound of Formula IVa-2 or IVb-2 is provided:

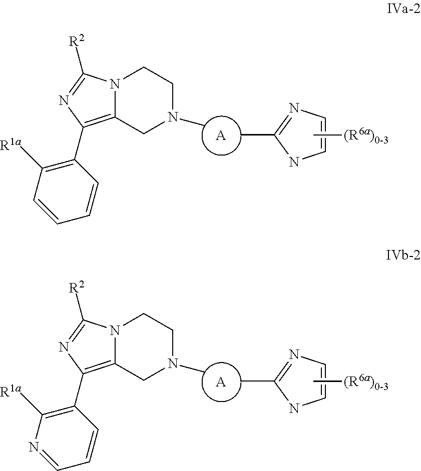

or a pharmaceutically acceptable salt thereof,
wherein Ring A, R^{1a}, R², and R^{6a} are as defined above and described in classes and subclasses herein, both singly and in combination.

In some embodiments, a compound of Formula Va or Vb is provided:

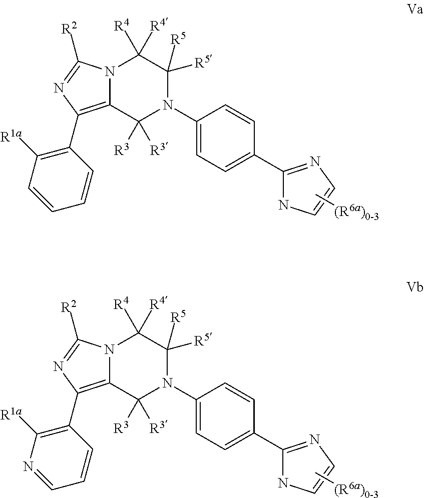

or a pharmaceutically acceptable salt thereof,
wherein R^{1a}, R², R³, R^{3′}, R⁴, R^{4′}, R⁵, R^{5′}, and R^{6a} are as defined above and described in classes and subclasses herein, both singly and in combination.

In some embodiments, a compound of Formula Va-1 or Vb-1 is provided:

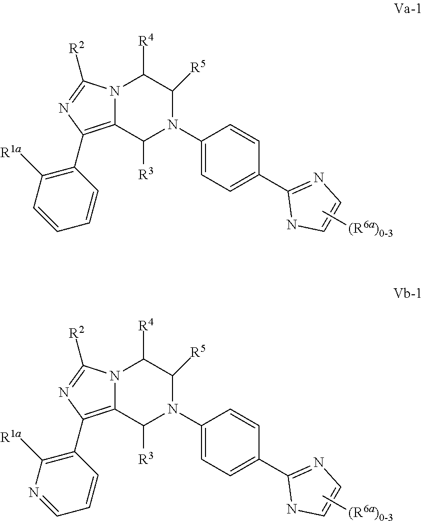

or a pharmaceutically acceptable salt thereof,
wherein R^{1a}, R², R³, R⁴, R⁵, and R^{6a} are as defined above and described in classes and subclasses herein, both singly and in combination.

In some embodiments, a compound of Formula Va-2 or Vb-2 is provided:

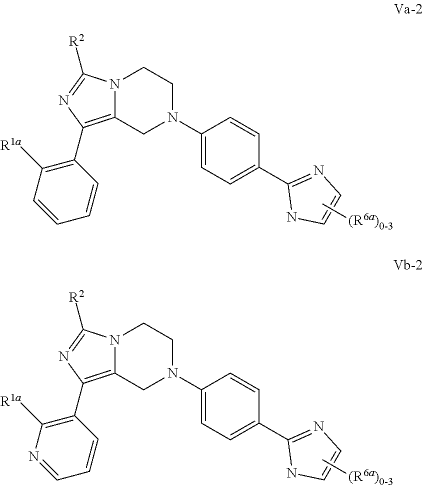

or a pharmaceutically acceptable salt thereof,
wherein R^{1a}, R², and R^{6a} are as defined above and described in classes and subclasses herein, both singly and in combination.

In some embodiments of any of Formulas I, IIa, IIb, IIIa, IIIb, IIIa-1, IIIb-1, IIIa-2, IIIb-2, IVa, IVb, IVa-1, IVb-1, IVa-2, and IVb-2, and as defined generally above, Ring A is —C₃-C₁₂ cycloalkyl, 3- to 14-membered heterocyclyl having 1 to 4 heteroatoms selected from oxygen, nitrogen, and sulfur, —C₆-C₁₀ aryl, or 5- to 14-membered heteroaryl having 1 to 4 heteroatoms selected from oxygen, nitrogen, and sulfur, wherein each cycloalkyl, heterocyclyl, aryl, or heteroaryl of Ring A is optionally substituted with one or more substituents selected from the group consisting of halogen, —C₁-C₆ alkyl, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, and —S(O)₂NR₂. In some embodiments, Ring A is —C₆-C₁₀ aryl or 5- to 14-membered heteroaryl having 1 to 4 heteroatoms selected from oxygen, nitrogen, and sulfur, wherein each aryl or heteroaryl of Ring A is optionally substituted with one or more substituents selected from the group consisting of halogen, —C₁-C₆ alkyl, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, and —S(O)₂NR₂.

In some embodiments, Ring A is —C₃-C₁₂ cycloalkyl. In some embodiments, Ring A is 3- to 14-membered heterocyclyl having 1 to 4 heteroatoms selected from oxygen, nitrogen, and sulfur.

In some embodiments, Ring A is —C₆-C₁₀ aryl optionally substituted with one or more substituents selected from the group consisting of halogen, —C₁-C₆ alkyl, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, and —S(O)₂NR₂. In some embodiments, Ring A is —C₆-C₁₀ aryl optionally substituted with one or more substituents selected from the group consisting of halogen or —C₁-C₆ alkyl.

In some embodiments, Ring A is —C₆-C₁₀ aryl optionally substituted with one or more halogen. In some embodiments, Ring A is —C₆-C₁₀ aryl optionally substituted with one or more substituents selected from bromo, chloro, or fluoro. In some embodiments, Ring A is —C₆-C₁₀ aryl optionally substituted with one or more fluoro.

In some embodiments, Ring A is —C₆ aryl optionally substituted with one or more substituents selected from the group consisting of halogen or —C₁-C₆ alkyl. In some embodiments, Ring A is phenyl optionally substituted with one or more substituents selected from the group consisting of halogen or —C₁-C₆ alkyl. In some embodiments, Ring A is phenyl optionally substituted with one or more substituents selected from halogen. In some embodiments, Ring A is phenyl optionally substituted with one or more substituents selected from bromo, chloro, or fluoro. In some embodiments, Ring A is phenyl optionally substituted with one or more fluoro.

In some embodiments, Ring A is —C₆-C₁₀ aryl optionally substituted with one or more substituents selected from —C₁-C₆ alkyl. In some embodiments, Ring A is —C₆-C₁₀ aryl optionally substituted with one or more substituents selected from —C₁-C₄ alkyl. In some embodiments, Ring A is —C₆-C₁₀ aryl optionally substituted with one or more substituents selected from —C₁-C₂ alkyl. In some embodiments, Ring A is —C₆-C₁₀ aryl optionally substituted with one or more substituents selected from methyl.

In some embodiments, Ring A is phenyl optionally substituted with one or more —C₁-C₆ alkyl. In some embodiments, Ring A is phenyl optionally substituted with one or more methyl.

In some embodiments, Ring A is 5- to 14-membered heteroaryl having 1 to 4 heteroatoms selected from oxygen, nitrogen, and sulfur optionally substituted with one or more substituents selected from the group consisting of halogen, —C₁-C₆ alkyl, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, and —S(O)₂NR₂.

In some embodiments, Ring A is a 6-membered heteroaryl having 1 to 4 heteroatoms selected from oxygen, nitrogen, and sulfur optionally substituted with one or more substituents selected from the group consisting of halogen or —C₁-C₆ alkyl. In some embodiments, Ring A is pyridyl, pyrimidyl, or pyrazinyl optionally optionally substituted with one or more substituents selected from the group consisting of halogen or —C₁-C₆ alkyl. In some embodiments, Ring A is pyridyl optionally substituted with one or more substituents selected from the group consisting of halogen or —C₁-C₆ alkyl. In some embodiments, Ring A is pyrimidinyl optionally substituted with one or more substituents selected from the group consisting of halogen or —C₁-C₆ alkyl. In some embodiments, Ring A is pyrazinyl optionally substituted with one or more substituents selected from the group consisting of halogen or —C₁-C₆ alkyl.

In some embodiments, Ring A is phenyl, pyridyl, pyrazinyl, or pyrimidinyl, wherein each phenyl, pyridyl, pyrazinyl, or pyrimidinyl of Ring A is optionally substituted with one or more substituents selected from the group consisting of halogen, —C₁-C₆ alkyl, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, and —S(O)₂NR₂.

In some embodiments, Ring A is phenyl, pyridyl, pyrazinyl, or pyrimidinyl, wherein each phenyl, pyridyl, pyrazinyl, or pyrimidinyl of Ring A is optionally substituted with one or more halogen.

In some embodiments, Ring A is phenyl, pyridyl, pyrazinyl, or pyrimidinyl, wherein each phenyl, pyridyl, pyrazinyl, or pyrimidinyl of Ring A is optionally substituted with one or more C₁-C₆ alkyl. In some embodiments, Ring A is phenyl pyridyl, pyrazinyl, or pyrimidinyl, wherein each phenyl, pyridyl, pyrazinyl, or pyrimidinyl of Ring A is optionally substituted with one or more methyl.

In some embodiments, Ring A is phenyl or pyridyl. In some embodiments, Ring A is phenyl. In some embodiments, Ring A is pyridyl.

In some embodiments, Ring A is selected from the group consisting of:

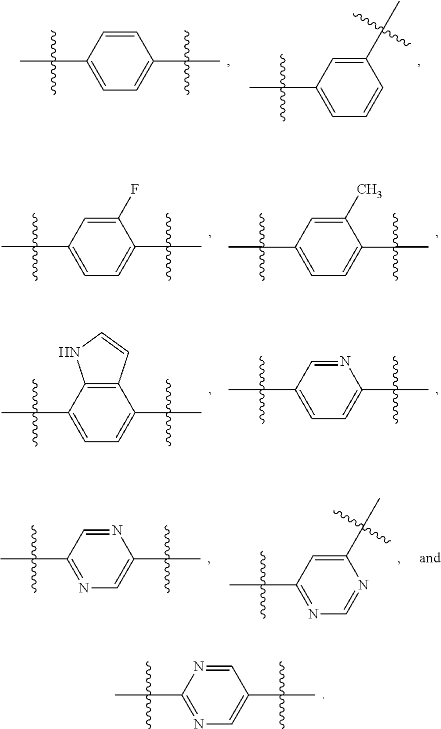

In some embodiments, Ring A is selected from the group consisting of:

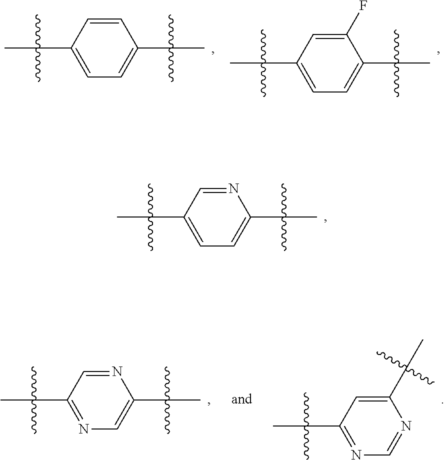 [SMILES: *c1ccc(*)cc1 ; *c1ccc(*)c(F)c1 ; *c1ccc(*)nc1 ; *c1cnc(*)cn1 ; *c1cc(*)ncn1]

In some embodiments, Ring A is selected from the group consisting of:

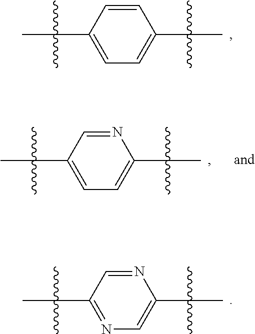 [SMILES: *c1ccc(*)cc1 ; *c1ccc(*)nc1 ; *c1cnc(*)cn1]

In some embodiments of any of Formulas I, IIa, IIb, IIIa, IIIb, IIIa-1, IIIb-1, IIIa-2, IIIb-2, IVa, IVb, IVa-1, IVb-1, IVa-2, IVb-2, Va, Vb, Va-1, Vb-1, Va-2, and Vb-2, and as defined generally above, R¹ is —C₆-C₁₀ aryl or 5- to 14-membered heteroaryl having 1 to 4 heteroatoms selected from oxygen, nitrogen, and sulfur optionally substituted with one or more R^{1a}. In some embodiments, R¹ is —C₆-C₁₀ aryl or 5- to 14-membered heteroaryl having 1 to 4 heteroatoms selected from oxygen, nitrogen, and sulfur optionally substituted with 0, 1, 2, 3, or 4 R^{1a}.

In some embodiments, R¹ is —C₆-C₁₀ aryl substituted with 0, 1, 2, 3, or 4 R^{1a}. In some embodiments R¹ is phenyl substituted with 0, 1, 2, 3, or 4 R^{1a}. In some embodiments, R¹ is phenyl substituted with 1 R^{a}. In some embodiments, R¹ is phenyl substituted with isopropyl.

In some embodiments, R¹ is 5- to 14-membered heteroaryl having 1 to 4 heteroatoms selected from oxygen, nitrogen, and sulfur substituted with 0, 1, 2, 3, or 4 R^{1a}. In some embodiments, R¹ is pyridyl, pyrimidinyl, or pyrazinyl substituted with 0, 1, 2, 3, or 4 R^{1a}. In some embodiments, R¹ is pyridyl substituted with 0, 1, 2, 3, or 4 R^{1a}. In some embodiments, R¹ is pyridyl substituted with 1 R^{1a}. In some embodiments, R¹ is pyridyl substituted with isopropyl. In some embodiments, R¹ is pyridyl substituted with —OCHF₂. In some embodiments, R¹ is pyridyl substituted with —OCH₂CF₃. In some embodiments, R¹ is pyridyl substituted with —OCH(CH₃)₂.

In some embodiments, R¹ is phenyl or 6-membered heteroaryl having 1 to 3 nitrogen atoms substituted with 0, 1, 2, 3, or 4 R^{1a}. In some embodiments, R¹ is phenyl or 6-membered heteroaryl having 1 to 3 nitrogen atoms substituted with 1 R^{1a}. In some embodiments, R¹ is phenyl or 6-membered heteroaryl having 1 to 3 nitrogen atoms substituted with isopropyl. In some embodiments, R¹ is phenyl or 6-membered heteroaryl having 1 to 3 nitrogen atoms substituted with —OCHF₂. In some embodiments, R¹ is phenyl or 6-membered heteroaryl having 1 to 3 nitrogen atoms substituted with —OCH₂CF₃. In some embodiments, R¹ is phenyl or 6-membered heteroaryl having 1 to 3 nitrogen atoms substituted with —OCH(CH₃)₂.

In some embodiments, R¹ is phenyl or pyridyl substituted with 0, 1, 2, 3, or 4 R^{1a}. In some embodiments, R¹ is phenyl or pyridyl substituted with 1 R^{1a}. In some embodiments, R¹ is phenyl or pyridyl substituted with isopropyl. In some embodiments, R¹ is phenyl or pyridyl substituted with —OCHF₂. In some embodiments, R¹ is phenyl or pyridyl substituted with —OCH₂CF₃. In some embodiments, R¹ is phenyl or pyridyl substituted with —OCH(CH₃)₂.

In some embodiments of any Formulas I, IIa, IIb, IIIa, IIIb, IIIa-1, IIIb-1, IIIa-2, IIIb-2, IVa, IVb, IVa-1, IVb-1, IVa-2, IVb-2, Va, Vb, Va-1, Vb-1, Va-2, and Vb-2, and as defined generally above, each R^{1a} is independently halogen, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, —C₁-C₆ alkyl, —C₂-C₆ alkenyl, —C₂-C₆ alkynyl, —C₃-C₁₂ cycloalkyl, —C₄-C₁₂ cycloalkenyl, 3- to 14-membered heterocyclyl having 1 to 4 heteroatoms selected from oxygen, nitrogen, or sulfur,—C₆—C₁₀ aryl, or 5- to 14-membered heteroaryl having 1 to 4 heteroatoms selected from oxygen, nitrogen, or sulfur.

In some embodiments, each R^{1a} is independently halogen, —OR, —NR₂, —CN, —NO₂, —SR, —C₁-C₆ alkyl, —C₃-C₆ cycloalkyl, or 3- to 6-membered heterocyclyl having 1 to 3 heteroatoms selected from oxygen, nitrogen, or sulfur, wherein each R of R^{1a} is independently —C₁-C₆ alkyl optionally substituted with one or more halogen.

In some embodiments, each R^{1a} is independently —OR or —C₁-C₆ alkyl, wherein each R of R^{1a} is independently —C₁-C₆ alkyl optionally substituted with one or more halogen. In some embodiments, each R^{1a} is independently —OR, wherein each R of R^{1a} is independently —C₁-C₆ alkyl optionally substituted with one or more halogen. In some embodiments, each R^{1a} is independently —OR, wherein each R of R^{1a} is independently —C₁-C₆ alkyl optionally substituted with one or more fluoro. In some embodiments, each R^{1a} is independently —OCHF₂, —OCH₂CF₃, or —OCH(CH₃)₂. In some embodiments, each R^{1a} is independently OCHF₂. In some embodiments, each R^{1a} is independently —OCH₂CF₃. In some embodiments, each R^{1a} is independently —OCH(CH₃)₂.

In some embodiments, each R^{1a} is independently —C₁-C₆ alkyl. In some embodiments, each R^{1a} is independently isopropyl.

In some embodiments, each R^{1a} is independently selected from the group consisting of isopropyl, —OCHF₂, —OCH₂CF₃, and —OCH(CH₃)₂.

In some embodiments of any of Formulas I, IIa, IIb, IIIa, IIIb, IIIa-1, IIIb-1, IIIa-2, IIIb-2, IVa, IVb, IVa-1, IVb-1, IVa-2, IVb-2, Va, Vb, Va-1, Vb-1, Va-2, and Vb-2, and as defined generally above, R² is —H, halogen, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, —C₁-C₆ alkyl, —C₂-C₆ alkenyl, —C₂-C₆ alkynyl, —C₃-C₁₂ cycloalkyl, —C₄-C₁₂ cycloalkenyl, 3- to 14-membered heterocyclyl having 1 to 4 heteroatoms selected from oxygen, nitrogen, or sulfur, —C₆-C₁₀ aryl, or 5- to 14-membered heteroaryl having 1 to 4 heteroatoms selected from oxygen, nitrogen, or sulfur, wherein each alkyl, alkenyl, alkynyl, cycloalkyl, cycloalkenyl, heterocyclyl, aryl, or heteroaryl of R² is optionally substituted with one or more halogen, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, —C₁-C₆ alkyl, —C₃-C₁₂ cycloalkyl, 3- to 14-membered heterocyclyl having 1 to 4 heteroatoms selected from oxygen, nitrogen, or sulfur, —C₆-C₁₀ aryl, or 5- to 14-membered heteroaryl having 1 to 4 heteroatoms selected from oxygen, nitrogen, or sulfur.

In some embodiments, R² is —H, halogen, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, —C₁-C₆ alkyl, —C₃-C₆ cycloalkyl, or 3- to 6-membered heterocyclyl having 1 to 3 heteroatoms selected from oxygen, nitrogen, or sulfur, wherein each alkyl, cycloalkyl, or heterocyclyl of R² is optionally substituted with one or more halogen, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, or —S(O)₂NR₂.

In some embodiments, R² is —H, —OR, —C₁-C₆ alkyl, or —C₃-C₁₂ cycloalkyl, wherein each alkyl or cycloalkyl of R² is optionally substituted with one or more halogen.

In some embodiments, R² is —H.

In some embodiments, R² is —OR. In some embodiments, R² is —OCH₃.

In some embodiments, R² is —C₁-C₆ alkyl optionally substituted with one or more halogen. In some embodiments, R² is methyl optionally substituted with one or more halogen. In some embodiments, R² is methyl optionally substituted with one or more fluoro. In some embodiments, R² is —CF₃.

In some embodiments, R² is —C₃-C₁₂ cycloalkyl optionally substituted with one or more halogen. In some embodiments, R² is —C₃-C₆ cycloalkyl optionally substituted with one or more halogen. In some embodiments, R² is cyclopropyl optionally substituted with one or more halogen. In some embodiments, R² is cyclopropyl optionally substituted with one or more fluoro.

In some embodiments, R² is selected from the group consisting of —H, methyl, —CF₃, —OCH₃, and cyclopropyl.

In some embodiments of any of Formulas I, IIa, IIb, IIIa, IIIb, IVa, IVb, Va, and Vb, and as defined generally above, each R³, R^{3′}, R⁴, R^{4′}, R⁵, and R^{5′} are independently selected from the group consisting of —H, —C₁-C₆ alkyl, —C₂-C₆ alkenyl, —C₂-C₆ alkynyl, and —C₃-C₇ cycloalkyl or each of (R³ and R^{3′}), or (R⁴ and R^{4′}), or (R⁵ and R^{5′}) can combine with the atom to which they are attached to form a —C₃-C₁₂ cycloalkyl ring or a 3- to 14-membered heterocyclyl ring having 1 to 4 heteroatoms selected from oxygen, nitrogen, or sulfur. In some embodiments, each R³, R^{3′}, R⁴, R^{4′}, R⁵, and R^{5′} are independently selected from the group consisting of —H, —C₁-C₆ alkyl, —C₂-C₆ alkenyl, —C₂-C₆ alkynyl, and —C₃-C₇ cycloalkyl.

In some embodiments, R³ is selected from the group consisting of —H, —C₁-C₆ alkyl, —C₂-C₆ alkenyl, —C₂-C₆ alkynyl, and —C₃-C₇ cycloalkyl.

In some embodiments, R^{3′} is selected from the group consisting of —H, —C₁-C₆ alkyl, —C₂-C₆ alkenyl, —C₂-C₆ alkynyl, and —C₃-C₇ cycloalkyl.

In some embodiments, R⁴ is selected from the group consisting of —H, —C₁-C₆ alkyl, —C₂-C₆ alkenyl, —C₂-C₆ alkynyl, and —C₃-C₇ cycloalkyl.

In some embodiments, R^{4′} is selected from the group consisting of —H, —C₁-C₆ alkyl, —C₂-C₆ alkenyl, —C₂-C₆ alkynyl, and —C₃-C₇ cycloalkyl.

In some embodiments, R⁵ is selected from the group consisting of —H, —C₁-C₆ alkyl, —C₂-C₆ alkenyl, —C₂-C₆ alkynyl, and —C₃-C₇ cycloalkyl.

In some embodiments, R^{5′} is selected from the group consisting of —H, —C₁-C₆ alkyl, —C₂-C₆ alkenyl, —C₂-C₆ alkynyl, and —C₃-C₇ cycloalkyl.

In some embodiments, each R³, R^{3′}, R⁴, R^{4′}, R⁵, and R^{5′} are independently selected from the group consisting of —H and —C₁-C₆ alkyl, or each of (R³ and R^{3′}), or (R⁴ and R^{4′}), or (R⁵ and R^{5′}) can combine with the atom to which they are attached to form a —C₃-C₆ cycloalkyl ring or a 3- to 6-membered heterocyclyl ring having 1 to 4 heteroatoms selected from oxygen, nitrogen, or sulfur.

In some embodiments, each R³, R^{3′}, R⁴, R^{4′}, R⁵, and R^{5′} are independently selected from the group consisting of —H and —C₁-C₆ alkyl. In some embodiments, R³, R^{3′}, R⁴, R^{4′}, R⁵, and R^{5′} are each —H.

In some embodiments, one of R³, R^{3′}, R⁴, R^{4′}, R⁵, and R^{5′} is methyl, and the others of R³, R^{3′}, R⁴, R^{4′}, R⁵, and R^{5′} are —H. In some embodiments, R³ is methyl, and R^{3′}, R⁴, R^{4′}, R⁵, and R^{5′} are —H. In some embodiments, R⁴ is methyl, and R³, R^{3′}, R^{4′}, R⁵, and R^{5′} are —H. In some embodiments, R⁵ is methyl, and R³, R^{3′}, R⁴, R^{4′}, and R^{5′} are —H.

In some embodiments of any of Formulas IIa-1, IIIb-1, IVa-1, IVb-1, Va-1, and Vb-1, and as defined generally above, R³, R⁴, and R⁵ are each independently selected from the group consisting of —H, —C₁-C₆ alkyl, —C₂-C₆ alkenyl, —C₂-C₆ alkynyl, and —C₃-C₇ cycloalkyl.

In some embodiments, R³ is selected from the group consisting of —H, —C₁-C₆ alkyl, —C₂-C₆ alkenyl, —C₂-C₆ alkynyl, and —C₃-C₇ cycloalkyl.

In some embodiments, R⁴ is selected from the group consisting of —H, —C₁-C₆ alkyl, —C₂-C₆ alkenyl, —C₂-C₆ alkynyl, and —C₃-C₇ cycloalkyl.

In some embodiments, R⁵ is selected from the group consisting of —H, —C₁-C₆ alkyl, —C₂-C₆ alkenyl, —C₂-C₆ alkynyl, and —C₃-C₇ cycloalkyl.

In some embodiments, R³, R⁴, and R⁵ are each independently —H or —C₁-C₆ alkyl. In some embodiments, R³, R⁴, and R⁵ are each —H. In some embodiments, R³ is —C₁-C₆ alkyl (e.g., methyl). In some embodiments, R⁴ is —C₁-C₆ alkyl (e.g., methyl). In some embodiments, R⁵ is —C₁-C₆ alkyl (e.g., methyl). In some embodiments, one of R³, R⁴, and R⁵ is methyl, and the others of R³, R⁴, and R⁵ are —H.

In some embodiments of any of Formulas I, IIa, IIb, IIIa, IIIb, IIIa-1, IIIb-1, IIIa-2, and IIIb-2, and as defined generally above, R⁶ is —H, halogen, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, —C₁-C₆ alkyl, —C₂-C₆ alkenyl, —C₂-C₆ alkynyl, —C₃-C₁₂ cycloalkyl, —C₄-C₁₂ cycloalkenyl, 3- to 14-membered heterocyclyl having 1 to 4 heteroatoms selected from oxygen, nitrogen, or sulfur, —C₆-C₁₀ aryl, or 5- to 14-membered heteroaryl having 1 to 4 heteroatoms selected from oxygen, nitrogen, or sulfur, wherein each alkyl, alkenyl, alkynyl, cycloalkyl, cycloalkenyl, heterocyclyl, aryl or heteroaryl of R⁶ is substituted with one or more R^{6a}. In some embodiments, R⁶ is —H, halogen, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, —C₁-C₆ alkyl, —C₂-C₆ alkenyl, —C₂-C₆ alkynyl, —C₃-C₁₂ cycloalkyl, —C₄-C₁₂ cycloalkenyl, 3- to 14-membered heterocyclyl having 1 to 4 heteroatoms selected from oxygen, nitrogen, or sulfur, —C₆-C₁₀ aryl, or 5- to 14-membered heteroaryl having 1 to 4 heteroatoms selected from oxygen, nitrogen, or sulfur, wherein each alkyl, alkenyl, alkynyl, cycloalkyl, cycloalkenyl, heterocyclyl, aryl or heteroaryl of R⁶ is substituted with 0, 1, 2, 3, or 4 R^{6a}.

In some embodiments, R⁶ is selected from the group consisting of —H, halogen, —OR, —NR₂, —NRC(O)R′, —CN, —C(O)NR₂, —C₁-C₆ alkyl, 3- to 14-membered heterocyclyl having 1 to 4 heteroatoms selected from oxygen, nitrogen, or sulfur, or 5- to 14-membered heteroaryl having 1 to 4 heteroatoms selected from oxygen, nitrogen, or sulfur, wherein each alkyl, heterocyclyl, and heteroaryl of R⁶ is substituted with 0, 1, 2, 3, or 4 R^{6a}.

In some embodiments, R⁶ is H.

In some embodiments, R⁶ is halogen. In some embodiments, R⁶ is bromo, chloro, or fluoro. In some embodiments, R⁶ is bromo. In some embodiments, R⁶ is chloro. In some embodiments, R⁶ is fluoro.

In some embodiments, R⁶ is —OR. In some embodiments, R⁶ is —OR, wherein R of R⁶ is —C₁-C₆ alkyl optionally substituted with 0, 1, 2, 3, or 4 R^{a}. In some embodiments, R⁶ is —OR, wherein R of R⁶ is —C₁-C₆ alkyl optionally substituted with 0 or 1 R^{a}. In some embodiments, R⁶ is —OR, wherein R of R⁶ is —C₁-C₆ alkyl optionally substituted with 5- to 6-membered heterocyclyl having 1 to 4 heteroatoms selected from oxygen, nitrogen, and sulfur. In some embodiments, R⁶ is —OR, wherein R of R⁶ is —C₁-C₆ alkyl. In some embodiments, R⁶ is —OCH₃ or

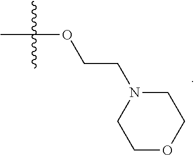 [SMILES: *OCCN1CCOCC1]

In some embodiments, R⁶ is —NR₂. In some embodiments, R⁶ is —NR₂, wherein each R of R⁶ is independently —H or —C₁-C₆ alkyl optionally substituted with 0, 1, 2, 3, or 4 R^{a}. In some embodiments, R⁶ is —NR₂, wherein each R of R⁶ is independently —C₁-C₆ alkyl. In some embodiments, R⁶ is —N(CH₃)₂.

In some embodiments, R⁶ is —NRC(O)R′. In some embodiments, R⁶ is —NHC(O)R′. In some embodiments, R⁶ is —NHC(O)R′, wherein R′ of R⁶ is —C₃-C₁₂ cycloalkyl. In some embodiments, R⁶ is —NHC(O)R′, wherein R′ of R⁶ is cyclopropyl.

In some embodiments, R⁶ is —C(O)NR₂. In some embodiments, R⁶ is —C(O)NR₂, wherein each R of R⁶ is independently selected from —H, —C₁-C₆ alkyl, —C₀-C₆ alkylene-C₆-C₁₀ aryl, and —C₃-C₁₂ cycloalkyl, wherein each alkyl, aryl, or cycloalkyl of R is substituted with 0, 1, 2, 3, or 4 R^{a}. In some embodiments, R⁶ is —C(O)NR₂, wherein two R groups of R⁶ combine with the atom to which they are attached to form a 5- to 6-membered heterocyclyl having 1 to 4 heteroatoms selected from oxygen, nitrogen, or sulfur, optionally substituted with —C₁-C₆ alkyl. In some embodiments, R⁶ is selected from:

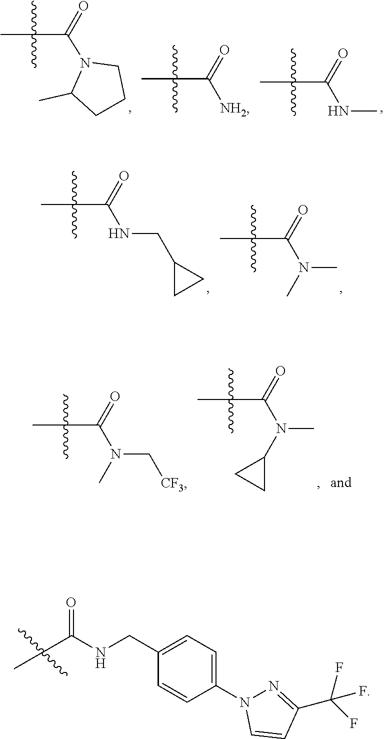

In some embodiments, R⁶ is C₁-C₆ alkyl, wherein alkyl of R⁶ is substituted with 0, 1, 2, 3, or 4 R^{6a}. In some embodiments, R⁶ is C₁-C₆ alkyl. In some embodiments, R⁶ is methyl.

In some embodiments, R⁶ is 3- to 14-membered heterocyclyl having 1 to 4 heteroatoms selected from oxygen, nitrogen, or sulfur, wherein heterocyclyl of R⁶ is substituted with 0, 1, 2, 3, or 4 R^{6a}. In some embodiments, R⁶ is 4- to 6-membered heterocyclyl having 1 to 2 heteroatoms selected from oxygen, nitrogen, or sulfur, wherein heterocyclyl of R⁶ is substituted with 0, 1, 2, 3, or 4 R^{6a}. In some embodiments, R⁶ is 4- to 6-membered heterocyclyl having 1 to 2 heteroatoms selected from oxygen, nitrogen, or sulfur, wherein heterocyclyl of R⁶ is substituted with 0 or 1 R^{6a}. In some embodiments, R⁶ is oxetanyl, dihydroisoxazolyl, pyrrolidinyl, piperidinyl, or morpholinyl, wherein R⁶ is substituted with 0 or 1 R^{6a}.

In some embodiments, R⁶ is selected from.

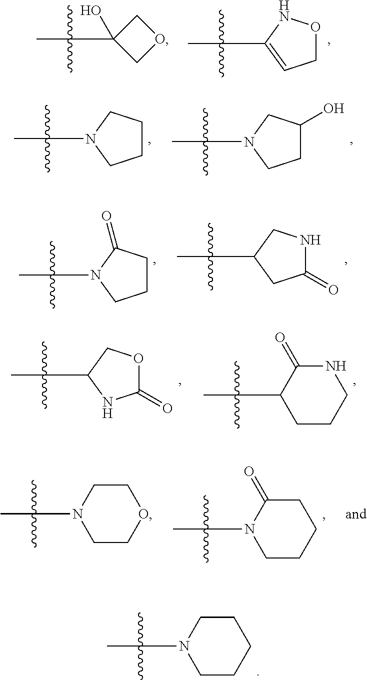

In some embodiments, R⁶ is 5- to 14-membered heteroaryl having 1 to 4 heteroatoms selected from oxygen, nitrogen, or sulfur, wherein heteroaryl of R⁶ is substituted with 0, 1, 2, 3 or 4 R^{6a}. In some embodiments, R⁶ is 5-membered heteroaryl having 1 to 4 heteroatoms selected from oxygen, nitrogen, or sulfur, wherein heteroaryl of R⁶ is substituted with 0, 1, 2, 3, or 4 R^{6a}. In some embodiments, R⁶ is pyrrolyl, imidazolyl, pyrazolyl, triazolyl, tetrazolyl, oxazolyl, or isoxazolyl substituted with 0, 1, 2, 3 or 4 R^{6a}. In some embodiments, R⁶ is imidazolyl substituted with 0, 1, 2, 3, or 4 R^{6a}. In some embodiments, R⁶ is 6-membered heteroaryl having 1 to 4 heteroatoms selected from oxygen, nitrogen, or sulfur, wherein heteroaryl of R⁶ is substituted with 0, 1, 2, 3, or 4 R^{6a}. In some embodiments, R⁶ is pyrazinyl or pyrimidinyl.

In some embodiments, R⁶ is selected from the group consisting of —NRC(O)R′, —C(O)NR₂, 3- to 14-membered heterocyclyl having 1 to 4 heteroatoms selected from oxygen, nitrogen, or sulfur, and 5- to 14-membered heteroaryl having 1 to 4 heteroatoms selected from oxygen, nitrogen, or sulfur, wherein each heterocyclyl or heteroaryl of R⁶ is substituted with 0, 1, 2, 3, or 4 R^{6a}.

In some embodiments, R⁶ is selected from the group consisting of

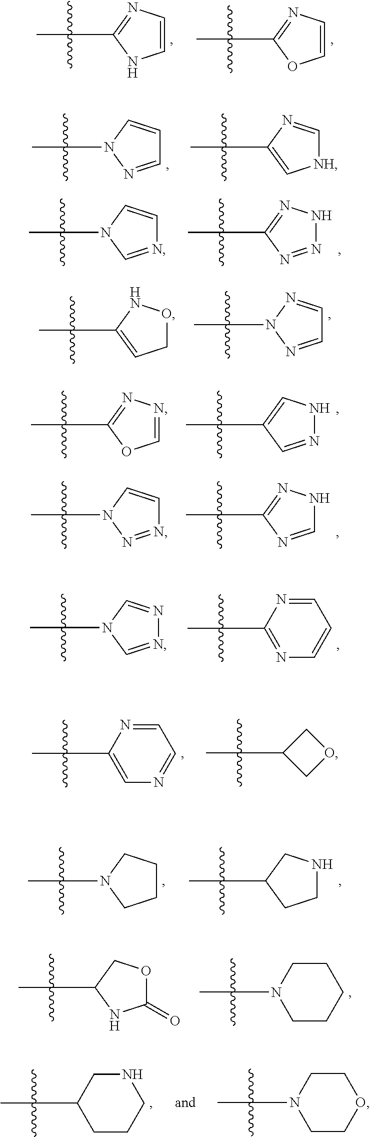

each of which is substituted with 0, 1, 2, 3, or 4 R^{6a}, or R⁶ is selected from the group consisting of —F, —CN, —OCH₃, —N(CH₃)₂, methyl,

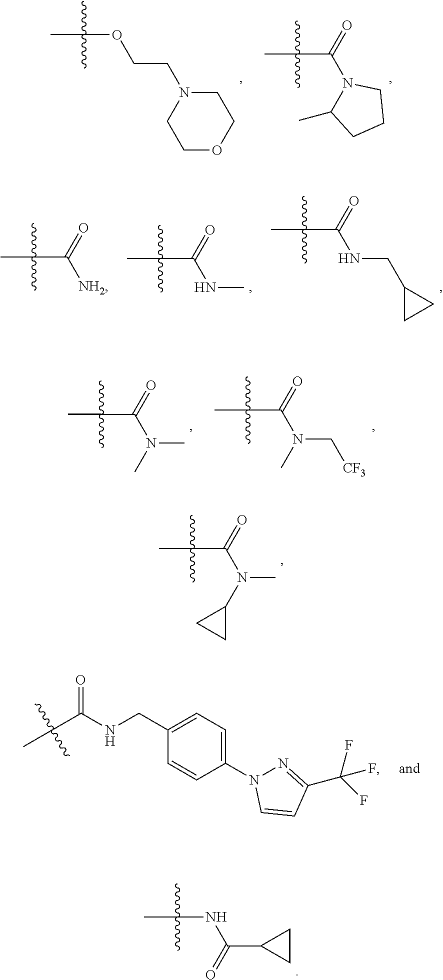

In some embodiments, R⁶ is selected from the group consisting of

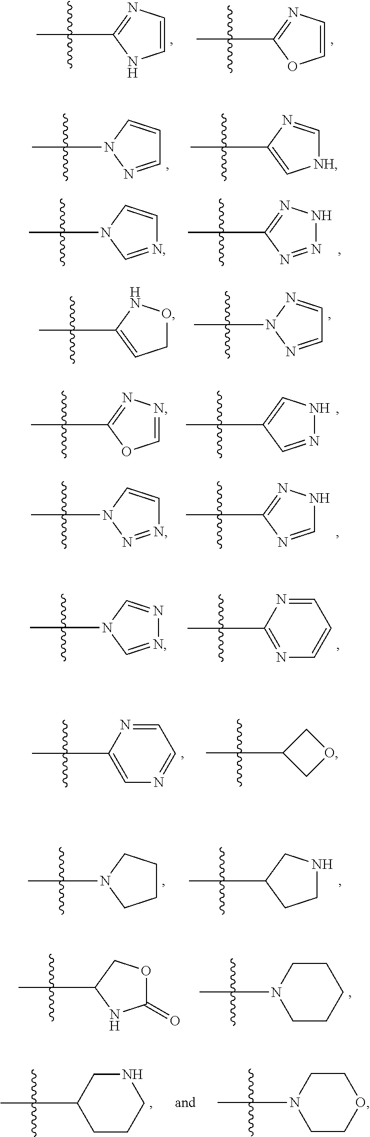

each of which is substituted with 0, 1, 2, 3, or 4 R^{6a}.

In some embodiments, R⁶ is selected from the group consisting of —F, —CN, —OCH₃, —N(CH₃)₂, methyl,

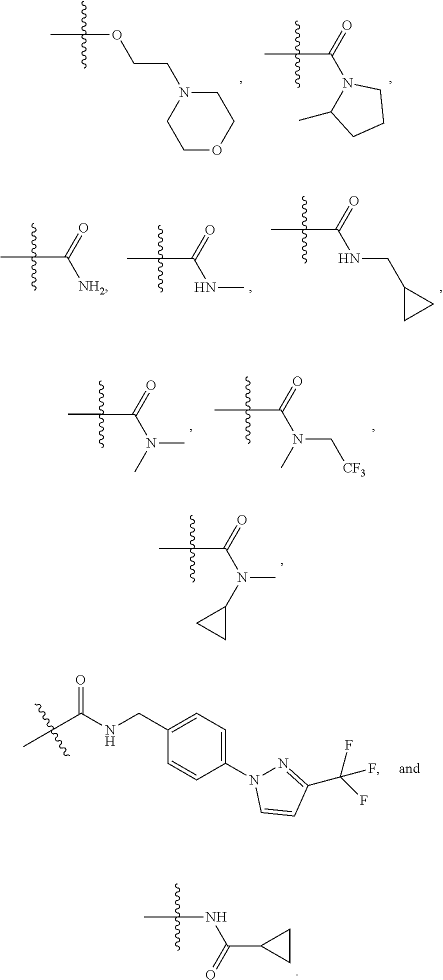

In some embodiments, R⁶ is selected from the group consisting of:

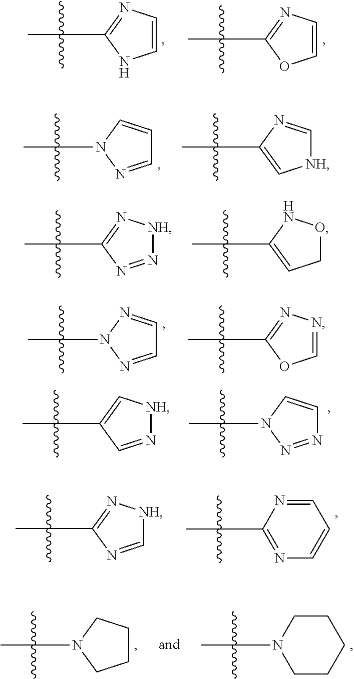

each of which is substituted with 0, 1, 2, 3, or 4 R^{6a}, or R⁶ is selected from the group consisting of methyl, —OCH₃,

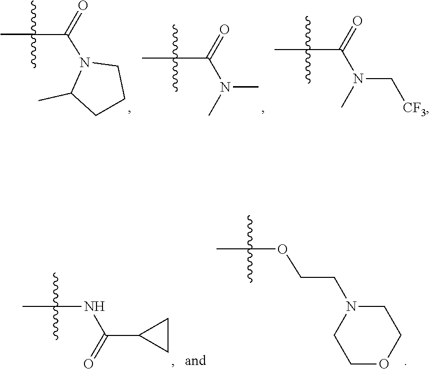

In some embodiments, R⁶ is selected from the group consisting of:

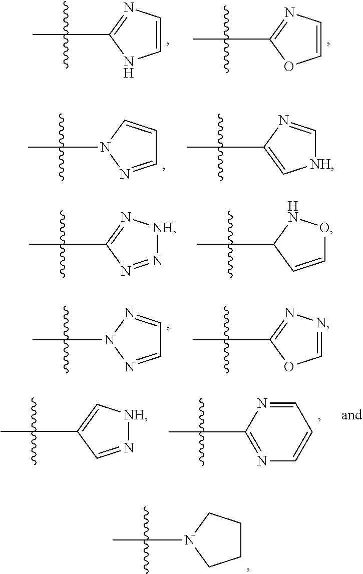

each of which is substituted with 0, 1, 2, 3, or 4 R^{6a}, or R⁶ is selected from the group consisting of:

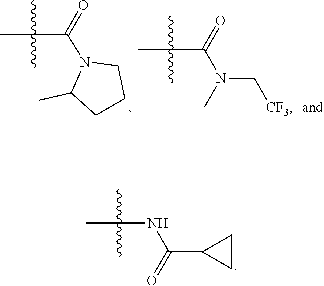

In some embodiments, R⁶ is substituted with 0, 1, 2, 3, or 4 R^{6a} and is selected from the group consisting of:

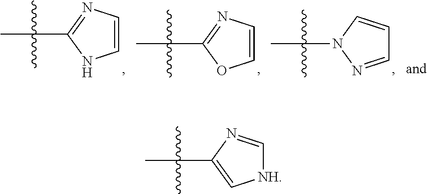 [SMILES: *c1ncc[nH]1 ; *c1ncco1 ; *n1cccn1 ; *c1c[nH]cn1]

In some embodiments, R⁶ is substituted with 0, 1, 2, 3, or 4 R^{6a} and is

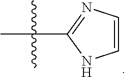 [SMILES: *c1ncc[nH]1]

In some embodiments, R⁶ is

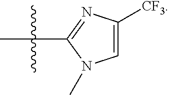 [SMILES: *c1nc(C(F)(F)F)cn1C]

In some embodiments of any of Formulas I, IIa, IIb, IIIa, IIIb, IIIa-1, IIIb-1, IIIa-2, IIIb-2, IVa, IVb, IVa-1, IVb-1, IVa-2, IVb-2, Va, Vb, Va-1, Vb-1, Va-2, and Vb-2, and as generally defined above, each R^{6a} is independently selected from the group consisting of halogen, oxo, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, —C₁-C₆ alkyl, —C₂-C₆ alkenyl, —C₂-C₆ alkynyl, —C₃-C₁₂ cycloalkyl, —C₄-C₁₂ cycloalkenyl, 3- to 14-membered heterocyclyl having 1 to 4 heteroatoms selected from oxygen, nitrogen, or sulfur, —C₆-C₁₀ aryl, and 5- to 14-membered heteroaryl having 1 to 4 heteroatoms selected from oxygen, nitrogen, or sulfur, wherein each alkyl, alkenyl, alkynyl, cycloalkyl, cycloalkenyl, heterocyclyl, aryl, or heteroaryl of R^{6a} is optionally substituted with one or more halogen, —C₁-C₆ alkyl, or —OR.

In some embodiments, each R^{6a} is independently selected from the group consisting of halogen, oxo, —OR, —C₁-C₆ alkyl, —C₃-C₁₂ cycloalkyl, and 3- to 14-membered heterocyclyl having 1 to 4 heteroatoms selected from oxygen, nitrogen, or sulfur, wherein each alkyl, cycloalkyl, or heterocyclyl of R^{6a} is optionally substituted with one or more halogen, —C₁-C₆ alkyl, or —OR. In some embodiments, each R^{6a} is independently selected from the group consisting of chloro, oxo, —OH, methyl, ethyl, isobutyl, cyclopropyl, azetidinyl, oxetanyl, and tetrahydropyranyl, wherein each methyl, ethyl, isobutyl, cyclopropyl, azetidinyl, oxetanyl, or tetrahydropyranyl of R^{6a} is optionally substituted with one or more fluoro, methyl, —OCH₃, or —OH.

In some embodiments, each R^{6a} is independently selected from the group consisting of halogen, —C₁-C₆ alkyl, —C₃-C₁₂ cycloalkyl, and 3- to 14-membered heterocyclyl having 1 to 4 heteroatoms selected from oxygen, nitrogen, or sulfur, wherein each alkyl, cycloalkyl, or heterocyclyl of R^{6a} is optionally substituted with one or more halogen, —C₁-C₆ alkyl, or —OR. In some embodiments, each R^{6a} is independently —C₁-C₆ alkyl optionally substituted with one or more halogen. In some embodiments, each R^{6a} is independently selected from the group consisting of chloro, methyl, ethyl, cyclopropyl, azetidinyl, and oxetanyl, wherein each methyl, ethyl, cyclopropyl, azetidinyl, or oxetanyl of R^{6a} is optionally substituted with one or more fluoro. In some embodiments, each R^{6a} is independently methyl or —CF₃.

In some embodiments, each R^{6a} is independently —C₁-C₆ alkyl. In some embodiments, each R^{6a} is independently —C₁-C₄ alkyl. In some embodiments, each R^{6a} is independently methyl. In some embodiments, each R^{6a} is independently isobutyl.

In some embodiments, each R^{6a} is independently —C₁-C₆ alkyl substituted with one or more halogen. In some embodiments, each R^{6a} is independently —C₁-C₄ alkyl substituted with one or more halogen. In some embodiments, each R^{6a} is independently —CF₃.

In some embodiments, each R^{6a} is independently —C₁-C₆ alkyl substituted with one or more —OR. In some embodiments, each R^{6a} is independently —C₁-C₆ alkyl substituted with one or more —OH. In some embodiments, each R^{6a} is independently —C₁-C₆ alkyl substituted with one or more —O(C₁-C₆ alkyl). In some embodiments, each R^{6a} is independently —C₁-C₆ alkyl substituted with one or more —OCH₃. In some embodiments, each R^{6a} is independently —C₁-C₄ alkyl substituted with one or more —OR. In some embodiments, each R^{6a} is independently —C₁-C₄ alkyl substituted with one or more —OH. In some embodiments, each R^{6a} is independently —C₁-C₄ alkyl substituted with one or more —O(C₁-C₆ alkyl). In some embodiments, each R^{6a} is independently —C₁-C₄ alkyl substituted with one or more —OCH₃. In some embodiments, each R^{6a} is independently —CH₂OH. In some embodiments, each R^{6a} is independently —CH₂CH₂OCH₃.

In some embodiments, each R^{6a} is independently halogen. In some embodiments, each R^{6a} is independently fluoro. In some embodiments, each R^{6a} is independently chloro. In some embodiments, each R^{6a} is independently bromo. In some embodiments, each R^{6a} is independently iodo.

In some embodiments, each R^{6a} is independently C₃-C₁₂ cycloalkyl, wherein each cycloalkyl of R^{6a} is optionally substituted with one or more halogen, —C₁-C₆ alkyl, or —OR. In some embodiments, each R^{6a} is independently C₃-C₆ cycloalkyl, wherein each cycloalkyl of R^{6a} is optionally substituted with one or more halogen, —C₁-C₆ alkyl, or —OR. In some embodiments, each R^{6a} is independently C₆ cycloalkyl, wherein each cycloalkyl of R^{6a} is optionally substituted with one or more halogen, —C₁-C₆ alkyl, or —OR. In some embodiments, each R^{6a} is independently C₅ cycloalkyl, wherein each cycloalkyl of R^{6a} is optionally substituted with one or more halogen, —C₁-C₆ alkyl, or —OR. In some embodiments, each R^{6a} is independently C₄ cycloalkyl, wherein each cycloalkyl of R^{6a} is optionally substituted with one or more halogen, —C₁-C₆ alkyl, or —OR. In some embodiments, each R^{6a} is independently C₃ cycloalkyl, wherein each cycloalkyl of R^{6a} is optionally substituted with one or more halogen, —C₁-C₆ alkyl, or —OR. In some embodiments, each R^{6a} is independently C₃ cycloalkyl.

In some embodiments, each R^{6a} is independently 3- to 14-membered heterocyclyl having 1 to 4 heteroatoms selected from oxygen, nitrogen, or sulfur, wherein each heterocyclyl of R^{6a} is optionally substituted with one or more halogen, —C₁-C₆ alkyl, or —OR. In some embodiments, each R^{6a} is independently 3- to 6-membered heterocyclyl having 1 to 3 heteroatoms selected from oxygen, nitrogen, or sulfur, wherein each heterocyclyl of R^{6a} is optionally substituted with one or more halogen, —C₁-C₆ alkyl, or —OR. In some embodiments, each R^{6a} is independently 6-membered heterocyclyl having 1-2 heteroatoms selected from oxygen, nitrogen, or sulfur, wherein each heterocyclyl of R^{6a} is optionally substituted with one or more halogen, —C₁-C₆ alkyl, or —OR. In some embodiments, each R^{6a} is independently 6-membered heterocyclyl having 1-2 heteroatoms selection from oxygen, nitrogen, or sulfur. In some embodiments, each R^{6a} is independently tetrahydropyranyl. In some embodiments, each R^{6a} is independently 5-membered heterocyclyl having 1-2 heteroatoms selected from oxygen, nitrogen, or sulfur, wherein each heterocyclyl of R^{6a} is optionally substituted with one or more halogen, —C₁-C₆ alkyl, or —OR. In some embodiments, each R^{6a} is independently 4-membered heterocyclyl having 1 heteroatom selected from oxygen, nitrogen, or sulfur, wherein each heterocyclyl of R^{6a} is optionally substituted with one or more halogen, —C₁-C₆ alkyl, or —OR. In some embodiments, each R^{6a} is independently oxetanyl. In some embodiments, each R^{6a} is independently azetidinyl optionally substituted with one or more C₁-C₆ alkyl. In some embodiments, each R^{6a} is independently azetidinyl substituted with methyl. In some embodiments, each R^{6a} is independently 3-membered heterocyclyl having 1 heteroatoms selected from oxygen, nitrogen, or sulfur, wherein each heterocyclyl of R^{6a} is optionally substituted with one or more halogen, —C₁-C₆ alkyl, or —OR.

In some embodiments, each R^{6a} is independently oxo.

In some embodiments, each R^{6a} is independently —OR. In some embodiments, each R^{6a} is independently —OH.

In some embodiments of Formulas I, IIa, IIb, IIIa, IIIb, IIIa-1, IIIb-1, IIIa-2, IIIb-2, IVa, IVb, IVa-1, IVb-1, IVa-2, IVb-2, Va, Vb, Va-1, Vb-1, Va-2, and Vb-2, and as defined generally above, each R is independently selected from the group consisting of —H, —C₁-C₆ alkyl, —C₀-C₆ alkylene-C₆-C₁₀ aryl, —C₃-C₁₂ cycloalkyl, 3- to 14-membered heterocyclyl having 1 to 4 heteroatoms selected from oxygen, nitrogen, or sulfur, or 5- to 14-membered heteroaryl having 1 to 4 heteroatoms selected from oxygen, nitrogen, or sulfur, wherein each alkyl, aryl, cycloalkyl, heterocyclyl, or heteroaryl of R is substituted with one or more R^{a}; or two R groups can combine with the atom to which they are attached to form a 5- to 6-membered heterocyclyl having 1 to 4 heteroatoms selected from oxygen, nitrogen, or sulfur, optionally substituted with —C₁-C₆ alkyl. In some embodiments, each R is independently selected from the group consisting of —H, —C₁-C₆ alkyl, —C₀-C₆ alkylene-C₆-C₁₀ aryl, —C₃-C₁₂ cycloalkyl, 3- to 14-membered heterocyclyl having 1 to 4 heteroatoms selected from oxygen, nitrogen, or sulfur, or 5- to 14-membered heteroaryl having 1 to 4 heteroatoms selected from oxygen, nitrogen, or sulfur, wherein each alkyl, aryl, cycloalkyl, heterocyclyl, or heteroaryl of R is substituted with 0, 1, 2, 3, or 4 R^{a}; or two R groups can combine with the atom to which they are attached to form a 5- to 6-membered heterocyclyl having 1 to 4 heteroatoms selected from oxygen, nitrogen, or sulfur, optionally substituted with —C₁-C₆ alkyl.

In some embodiments, each R is independently selected from the group consisting of —H, —C₁-C₆ alkyl, —C₀-C₆ alkylene-C₆-C₁₀ aryl, and —C₃-C₁₂ cycloalkyl, wherein each alkyl, aryl, or cycloalkyl of R is substituted with 0, 1, 2, 3, or 4 R^{a}; or two R groups can combine with the atom to which they are attached to form a 5- to 6-membered heterocyclyl having 1 to 4 heteroatoms selected from oxygen, nitrogen, or sulfur, optionally substituted with —C₁-C₆ alkyl. In some embodiments, each R is independently selected from the group consisting of —H, methyl, ethyl, isopropyl, —CH₂-phenyl, and cyclopropyl, wherein each methyl, ethyl, isopropyl, phenyl, or cyclopropyl of R is substituted with 0, 1, 2, 3, or 4 R^{a}; or two R groups can combine with the atom to which they are attached to form a pyrrolidinyl, optionally substituted with methyl.

In some embodiments, each R is independently selected from the group consisting of —H and —C₁-C₆ alkyl (e.g., methyl, ethyl, or isopropyl), wherein each alkyl of R is substituted with 0, 1, 2, 3, or 4 R^{a}. In some embodiments, each R is independently selected from the group consisting of —H, methyl, isopropyl, —CHF₂, and —CH₂CF₃.

In some embodiments of Formulas I, IIa, IIb, IIIa, IIIb, IIIa-1, IIIb-1, IIIa-2, IIIb-2, IVa, IVb, IVa-1, IVb-1, IVa-2, IVb-2, Va, Vb, Va-1, Vb-1, Va-2, and Vb-2, and as defined generally above, each R^{a} is independently halogen, —O(C₁-C₆ alkyl), —NH(C₁-C₆ alkyl), —N(C₁-C₆ alkyl)₂, —C₃-C₆ cycloalkyl, 5- to 6-membered heterocyclyl having 1 to 4 heteroatoms selected from oxygen, nitrogen, or sulfur, or 5- to 6-membered heteroaryl having 1 to 4 heteroatoms selected from oxygen, nitrogen, or sulfur, wherein each cycloalkyl, heterocyclyl, or heteroaryl of R^{a} is optionally substituted with —C₁-C₆ alkyl or —C₁-C₆ alkyl substituted with one or more halogen.

In some embodiments, each R^{a} is independently halogen, —C₃-C₆ cycloalkyl, 5- to 6-membered heterocyclyl having 1 to 4 heteroatoms selected from oxygen, nitrogen, or sulfur, or 5- to 6-membered heteroaryl having 1 to 4 heteroatoms selected from oxygen, nitrogen, or sulfur, wherein each cycloalkyl, heterocyclyl, or heteroaryl of R^{a} is optionally substituted with —C₁-C₆ alkyl or —C₁-C₆ alkyl substituted with one or more halogen. In some embodiments, each R^{a} is independently selected from the group consisting of fluoro, cyclopropyl, morpholinyl, or pyrazolyl, wherein each cyclopropyl, morpholinyl, or pyrazolyl of R^{a} is optionally substituted with —CF₃.

In some embodiments, each R^{a} is independently selected from the group consisting of halogen, 5- to 6-membered heterocyclyl having 1 to 4 heteroatoms selected from oxygen, nitrogen, or sulfur, or 5- to 6-membered heteroaryl having 1 to 4 heteroatoms selected from oxygen, nitrogen, or sulfur, wherein each heteroaryl of R^{a} is optionally substituted with —C₁-C₆ alkyl substituted with one or more halogen. In some embodiments, each R^{a} is independently selected from the group consisting of fluoro, morpholinyl, or pyrazolyl, wherein each morpholinyl or pyrazolyl of R^{a} is optionally substituted with —CF₃.

In some embodiments of Formulas I, IIa, IIb, IIIa, IIIb, IIIa-1, IIIb-1, IIIa-2, IIIb-2, IVa, IVb, IVa-1, IVb-1, IVa-2, IVb-2, Va, Vb, Va-1, Vb-1, Va-2, and Vb-2, and as defined generally above, each R′ is independently selected from the group consisting of —C₁-C₆ alkyl, —C₂-C₆ alkenyl, —C₂-C₆ alkynyl, —C₃-C₁₂ cycloalkyl, —C₄-C₁₂ cycloalkenyl, 3- to 14-membered heterocyclyl having 1 to 4 heteroatoms selected from oxygen, nitrogen, or sulfur, —C₆-C₁₀ aryl, and 5- to 14-membered heteroaryl having 1 to 4 heteroatoms selected from oxygen, nitrogen, or sulfur.

In some embodiments, each R′ is independently selected from the group consisting of —C₁-C₆ alkyl and —C₃-C₁₂ cycloalkyl. In some embodiments, each R′ is independently —C₃-C₁₂ cycloalkyl. In some embodiments, each R′ is cyclopropyl.

In some embodiments, the present disclosure provides a compound, or a pharmaceutically acceptable salt thereof, selected from Table 1.

TABLE 1

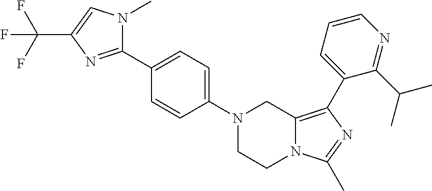 [SMILES: Cc1nc(-c2cccnc2C(C)C)c2n1CCN(c1ccc(-c3nc(C(F)(F)F)cn3C)cc1)C2]

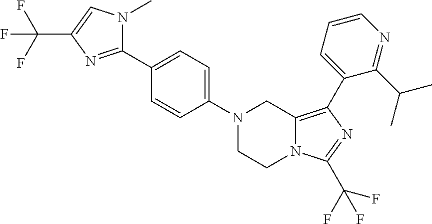 [SMILES: CC(C)c1ncccc1-c1nc(C(F)(F)F)n2c1CN(c1ccc(-c3nc(C(F)(F)F)cn3C)cc1)CC2]

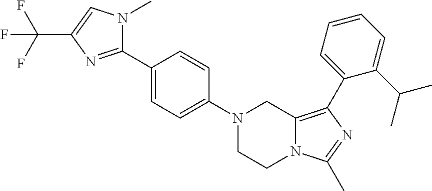 [SMILES: Cc1nc(-c2ccccc2C(C)C)c2n1CCN(c1ccc(-c3nc(C(F)(F)F)cn3C)cc1)C2]

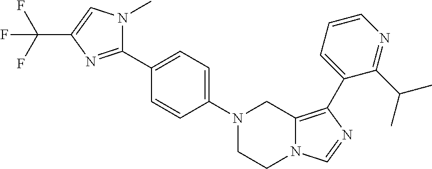 [SMILES: CC(C)c1ncccc1-c1ncn2c1CN(c1ccc(-c3nc(C(F)(F)F)cn3C)cc1)CC2]

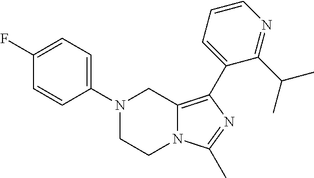 [SMILES: Cc1nc(-c2cccnc2C(C)C)c2n1CCN(c1ccc(F)cc1)C2]

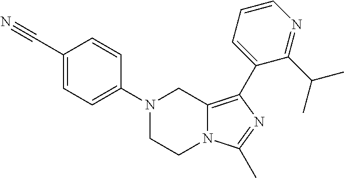 [SMILES: Cc1nc(-c2cccnc2C(C)C)c2n1CCN(c1ccc(C#N)cc1)C2]

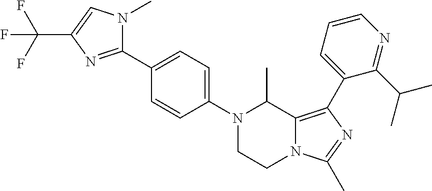 [SMILES: Cc1nc(-c2cccnc2C(C)C)c2n1CCN(c1ccc(-c3nc(C(F)(F)F)cn3C)cc1)C2C]

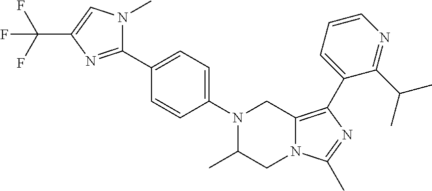 [SMILES: Cc1nc(-c2cccnc2C(C)C)c2n1CC(C)N(c1ccc(-c3nc(C(F)(F)F)cn3C)cc1)C2]

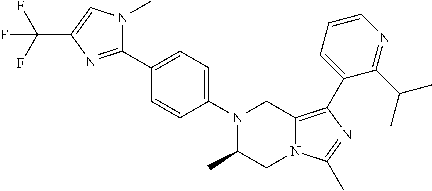 [SMILES: Cc1nc(-c2cccnc2C(C)C)c2n1C[C@@H](C)N(c1ccc(-c3nc(C(F)(F)F)cn3C)cc1)C2]

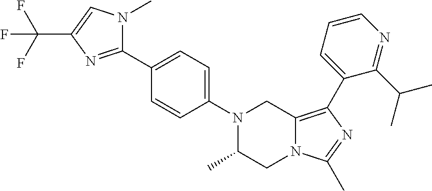 [SMILES: Cc1nc(-c2cccnc2C(C)C)c2n1C[C@H](C)N(c1ccc(-c3nc(C(F)(F)F)cn3C)cc1)C2]

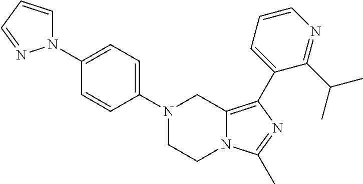 [SMILES: Cc1nc(-c2cccnc2C(C)C)c2n1CCN(c1ccc(-n3cccn3)cc1)C2]

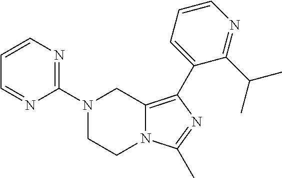 [SMILES: Cc1nc(-c2cccnc2C(C)C)c2n1CCN(c1ncccn1)C2]

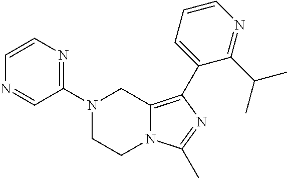 [SMILES: Cc1nc(-c2cccnc2C(C)C)c2n1CCN(c1cnccn1)C2]

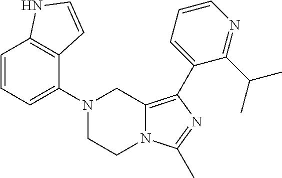 [SMILES: Cc1nc(-c2cccnc2C(C)C)c2n1CCN(c1cccc3[nH]ccc13)C2]

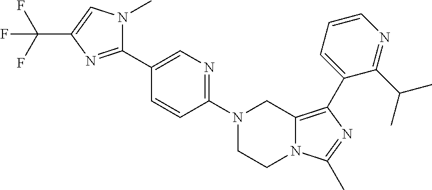 [SMILES: Cc1nc(-c2cccnc2C(C)C)c2n1CCN(c1ccc(-c3nc(C(F)(F)F)cn3C)cn1)C2]

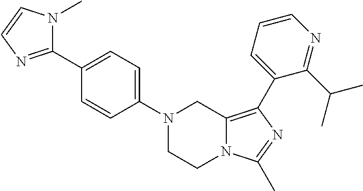 [SMILES: Cc1nc(-c2cccnc2C(C)C)c2n1CCN(c1ccc(-c3nccn3C)cc1)C2]

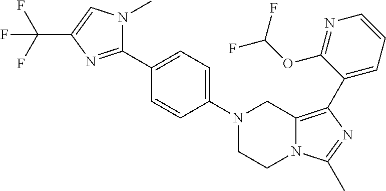 [SMILES: Cc1nc(-c2cccnc2OC(F)F)c2n1CCN(c1ccc(-c3nc(C(F)(F)F)cn3C)cc1)C2]

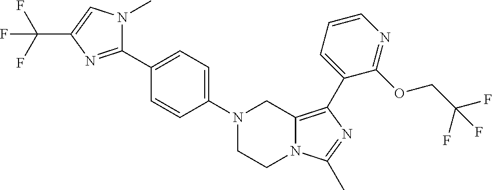 [SMILES: Cc1nc(-c2cccnc2OCC(F)(F)F)c2n1CCN(c1ccc(-c3nc(C(F)(F)F)cn3C)cc1)C2]

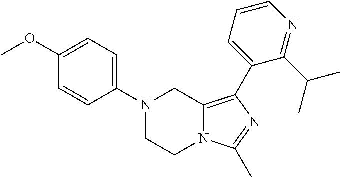 [SMILES: COc1ccc(N2CCn3c(C)nc(-c4cccnc4C(C)C)c3C2)cc1]

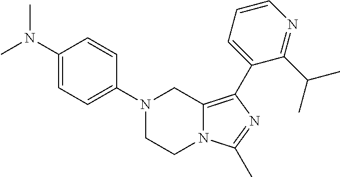 [SMILES: Cc1nc(-c2cccnc2C(C)C)c2n1CCN(c1ccc(N(C)C)cc1)C2]

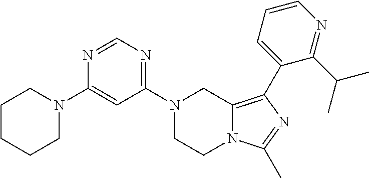 [SMILES: Cc1nc(-c2cccnc2C(C)C)c2n1CCN(c1cc(N3CCCCC3)ncn1)C2]

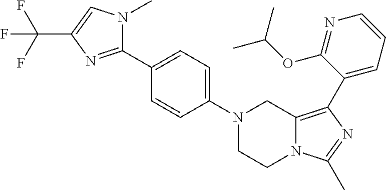 [SMILES: Cc1nc(-c2cccnc2OC(C)C)c2n1CCN(c1ccc(-c3nc(C(F)(F)F)cn3C)cc1)C2]

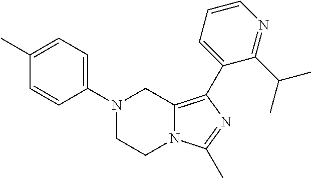 [SMILES: Cc1ccc(N2CCn3c(C)nc(-c4cccnc4C(C)C)c3C2)cc1]

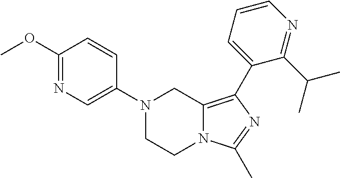 [SMILES: COc1ccc(N2CCn3c(C)nc(-c4cccnc4C(C)C)c3C2)cn1]

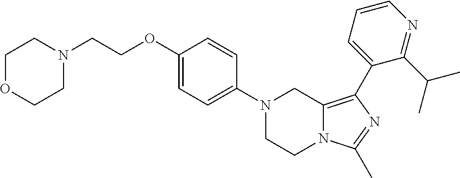 [SMILES: Cc1nc(-c2cccnc2C(C)C)c2n1CCN(c1ccc(OCCN3CCOCC3)cc1)C2]

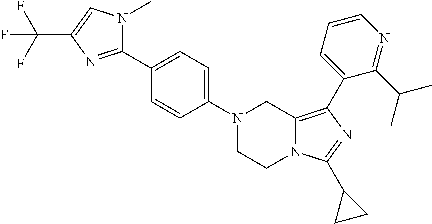 [SMILES: CC(C)c1ncccc1-c1nc(C2CC2)n2c1CN(c1ccc(-c3nc(C(F)(F)F)cn3C)cc1)CC2]

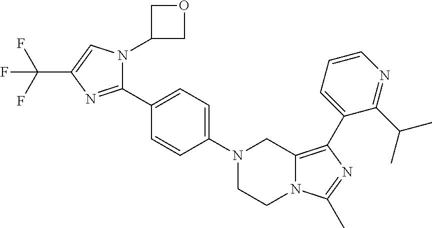 [SMILES: Cc1nc(-c2cccnc2C(C)C)c2n1CCN(c1ccc(-c3nc(C(F)(F)F)cn3C3COC3)cc1)C2]

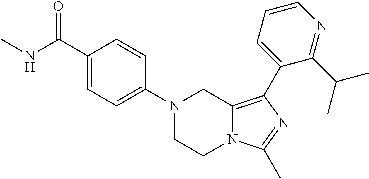 [SMILES: CNC(=O)c1ccc(N2CCn3c(C)nc(-c4cccnc4C(C)C)c3C2)cc1]

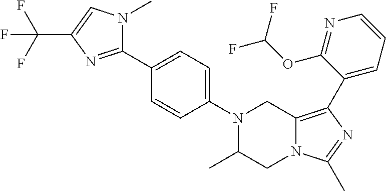 [SMILES: Cc1nc(-c2cccnc2OC(F)F)c2n1CC(C)N(c1ccc(-c3nc(C(F)(F)F)cn3C)cc1)C2]

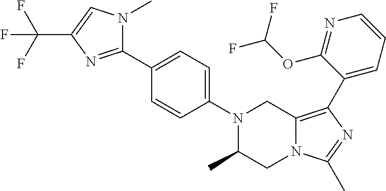 [SMILES: Cc1nc(-c2cccnc2OC(F)F)c2n1C[C@@H](C)N(c1ccc(-c3nc(C(F)(F)F)cn3C)cc1)C2]

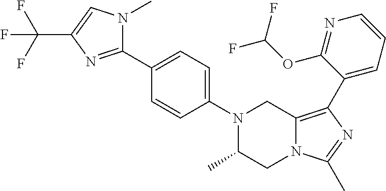 [SMILES: Cc1nc(-c2cccnc2OC(F)F)c2n1C[C@H](C)N(c1ccc(-c3nc(C(F)(F)F)cn3C)cc1)C2]

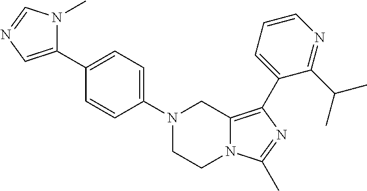 [SMILES: Cc1nc(-c2cccnc2C(C)C)c2n1CCN(c1ccc(-c3cncn3C)cc1)C2]

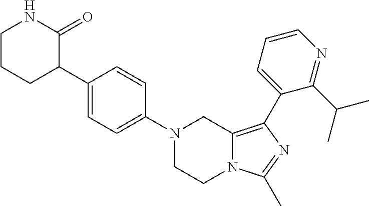 [SMILES: Cc1nc(-c2cccnc2C(C)C)c2n1CCN(c1ccc(C3CCCNC3=O)cc1)C2]

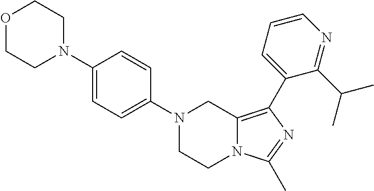 [SMILES: Cc1nc(-c2cccnc2C(C)C)c2n1CCN(c1ccc(N3CCOCC3)cc1)C2]

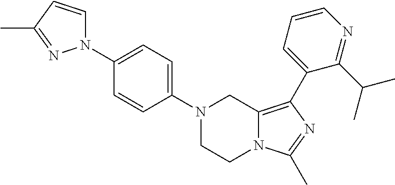 [SMILES: Cc1ccn(-c2ccc(N3CCn4c(C)nc(-c5cccnc5C(C)C)c4C3)cc2)n1]

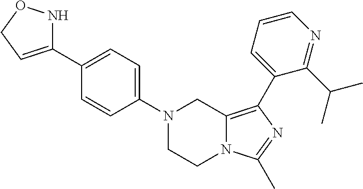 [SMILES: Cc1nc(-c2cccnc2C(C)C)c2n1CCN(c1ccc(C3=CCON3)cc1)C2]

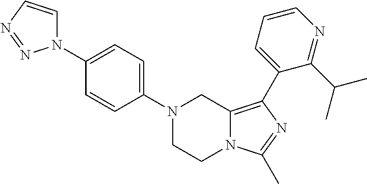 [SMILES: Cc1nc(-c2cccnc2C(C)C)c2n1CCN(c1ccc(-n3ccnn3)cc1)C2]

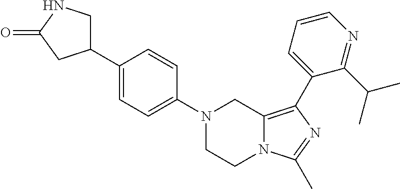 [SMILES: Cc1nc(-c2cccnc2C(C)C)c2n1CCN(c1ccc(C3CNC(=O)C3)cc1)C2]

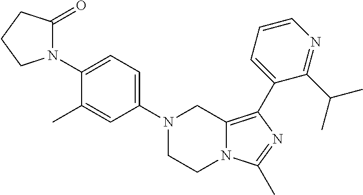 [SMILES: Cc1cc(N2CCn3c(C)nc(-c4cccnc4C(C)C)c3C2)ccc1N1CCCC1=O]

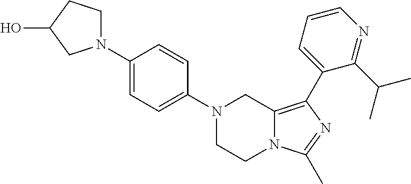 [SMILES: Cc1nc(-c2cccnc2C(C)C)c2n1CCN(c1ccc(N3CCC(O)C3)cc1)C2]

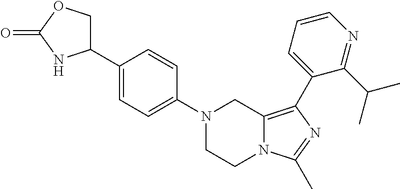 [SMILES: Cc1nc(-c2cccnc2C(C)C)c2n1CCN(c1ccc(C3COC(=O)N3)cc1)C2]

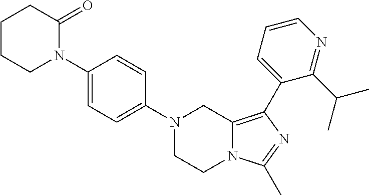 [SMILES: Cc1nc(-c2cccnc2C(C)C)c2n1CCN(c1ccc(N3CCCCC3=O)cc1)C2]

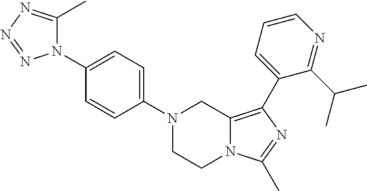 [SMILES: Cc1nnnn1-c1ccc(N2CCn3c(C)nc(-c4cccnc4C(C)C)c3C2)cc1]

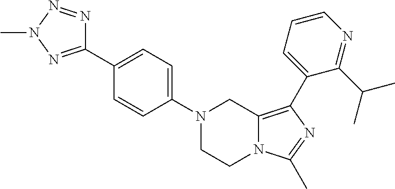 [SMILES: Cc1nc(-c2cccnc2C(C)C)c2n1CCN(c1ccc(-c3nnn(C)n3)cc1)C2]

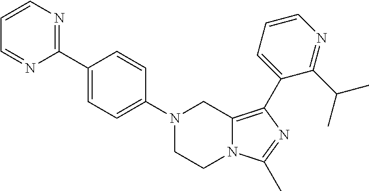 [SMILES: Cc1nc(-c2cccnc2C(C)C)c2n1CCN(c1ccc(-c3ncccn3)cc1)C2]

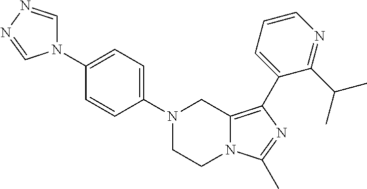 [SMILES: Cc1nc(-c2cccnc2C(C)C)c2n1CCN(c1ccc(-n3cnnc3)cc1)C2]

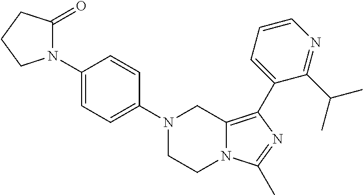 [SMILES: Cc1nc(-c2cccnc2C(C)C)c2n1CCN(c1ccc(N3CCCC3=O)cc1)C2]

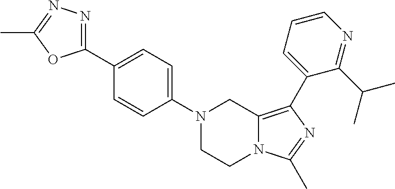 [SMILES: Cc1nnc(-c2ccc(N3CCn4c(C)nc(-c5cccnc5C(C)C)c4C3)cc2)o1]

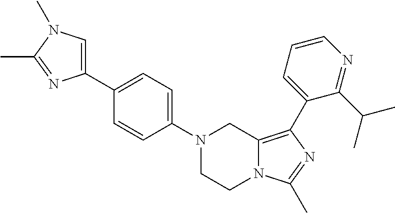 [SMILES: Cc1nc(-c2ccc(N3CCn4c(C)nc(-c5cccnc5C(C)C)c4C3)cc2)cn1C]

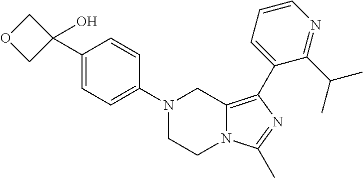 [SMILES: Cc1nc(-c2cccnc2C(C)C)c2n1CCN(c1ccc(C3(O)COC3)cc1)C2]

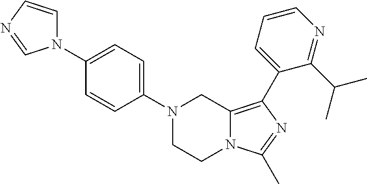 [SMILES: Cc1nc(-c2cccnc2C(C)C)c2n1CCN(c1ccc(-n3ccnc3)cc1)C2]

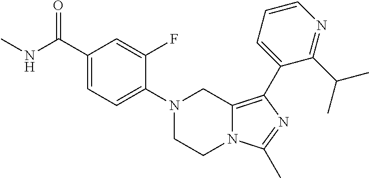 [SMILES: CNC(=O)c1ccc(N2CCn3c(C)nc(-c4cccnc4C(C)C)c3C2)c(F)c1]

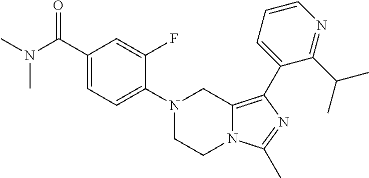 [SMILES: Cc1nc(-c2cccnc2C(C)C)c2n1CCN(c1ccc(C(=O)N(C)C)cc1F)C2]

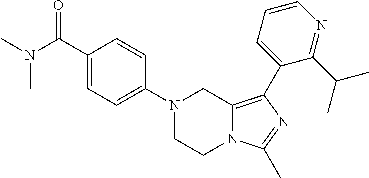 [SMILES: Cc1nc(-c2cccnc2C(C)C)c2n1CCN(c1ccc(C(=O)N(C)C)cc1)C2]

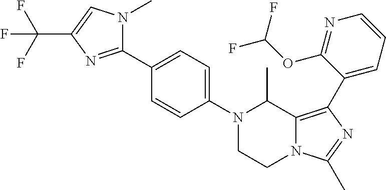 [SMILES: Cc1nc(-c2cccnc2OC(F)F)c2n1CCN(c1ccc(-c3nc(C(F)(F)F)cn3C)cc1)C2C]

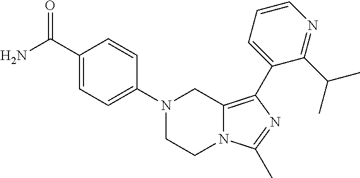 [SMILES: Cc1nc(-c2cccnc2C(C)C)c2n1CCN(c1ccc(C(N)=O)cc1)C2]

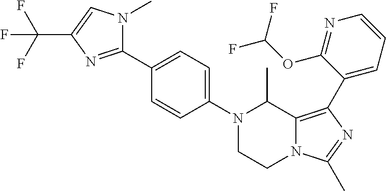 [SMILES: Cc1nc(-c2cccnc2OC(F)F)c2n1CCN(c1ccc(-c3nc(C(F)(F)F)cn3C)cc1)C2C]

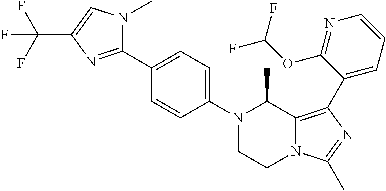 [SMILES: Cc1nc(-c2cccnc2OC(F)F)c2n1CCN(c1ccc(-c3nc(C(F)(F)F)cn3C)cc1)[C@H]2C]

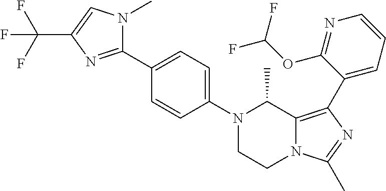 [SMILES: Cc1nc(-c2cccnc2OC(F)F)c2n1CCN(c1ccc(-c3nc(C(F)(F)F)cn3C)cc1)[C@@H]2C]

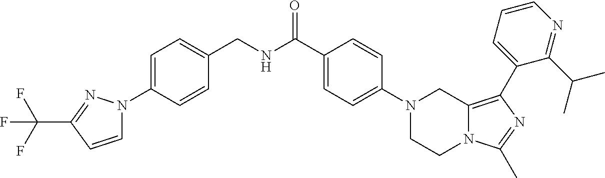 [SMILES: Cc1nc(-c2cccnc2C(C)C)c2n1CCN(c1ccc(C(=O)NCc3ccc(-n4ccc(C(F)(F)F)n4)cc3)cc1)C2]

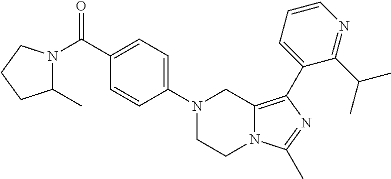 [SMILES: Cc1nc(-c2cccnc2C(C)C)c2n1CCN(c1ccc(C(=O)N3CCCC3C)cc1)C2]

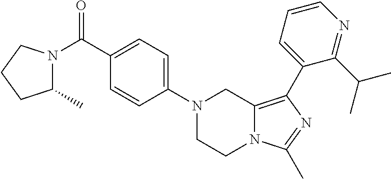 [SMILES: Cc1nc(-c2cccnc2C(C)C)c2n1CCN(c1ccc(C(=O)N3CCC[C@H]3C)cc1)C2]

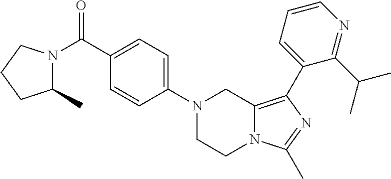 [SMILES: Cc1nc(-c2cccnc2C(C)C)c2n1CCN(c1ccc(C(=O)N3CCC[C@@H]3C)cc1)C2]

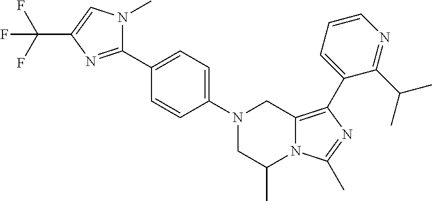 [SMILES: Cc1nc(-c2cccnc2C(C)C)c2n1C(C)CN(c1ccc(-c3nc(C(F)(F)F)cn3C)cc1)C2]

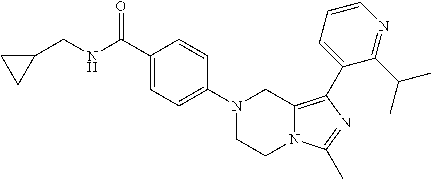 [SMILES: Cc1nc(-c2cccnc2C(C)C)c2n1CCN(c1ccc(C(=O)NCC3CC3)cc1)C2]

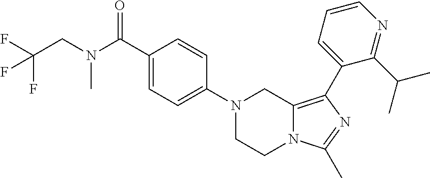 [SMILES: Cc1nc(-c2cccnc2C(C)C)c2n1CCN(c1ccc(C(=O)N(C)CC(F)(F)F)cc1)C2]

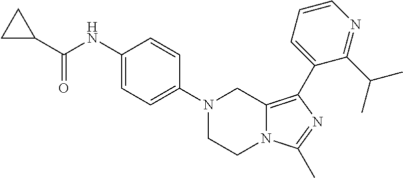 [SMILES: Cc1nc(-c2cccnc2C(C)C)c2n1CCN(c1ccc(NC(=O)C3CC3)cc1)C2]

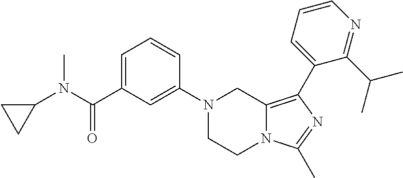 [SMILES: Cc1nc(-c2cccnc2C(C)C)c2n1CCN(c1cccc(C(=O)N(C)C3CC3)c1)C2]

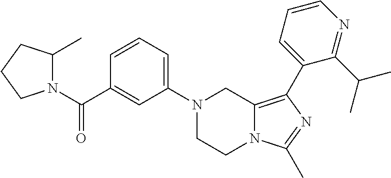 [SMILES: Cc1nc(-c2cccnc2C(C)C)c2n1CCN(c1cccc(C(=O)N3CCCC3C)c1)C2]

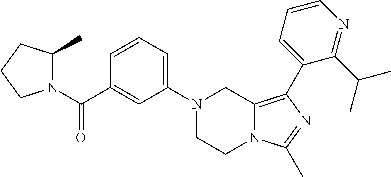 [SMILES: Cc1nc(-c2cccnc2C(C)C)c2n1CCN(c1cccc(C(=O)N3CCC[C@H]3C)c1)C2]

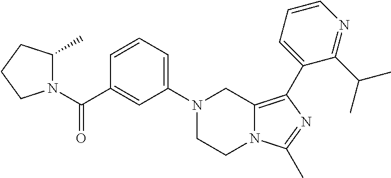 [SMILES: Cc1nc(-c2cccnc2C(C)C)c2n1CCN(c1cccc(C(=O)N3CCC[C@@H]3C)c1)C2]

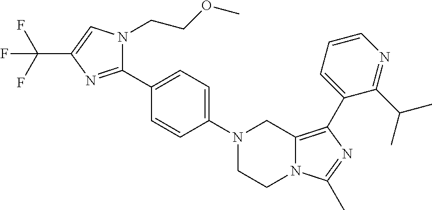 [SMILES: COCCn1cc(C(F)(F)F)nc1-c1ccc(N2CCn3c(C)nc(-c4cccnc4C(C)C)c3C2)cc1]

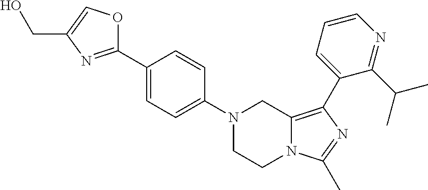 [SMILES: Cc1nc(-c2cccnc2C(C)C)c2n1CCN(c1ccc(-c3nc(CO)co3)cc1)C2]

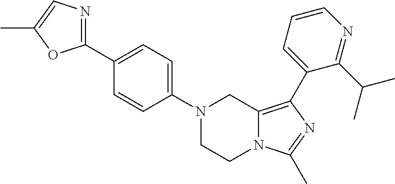 [SMILES: Cc1cnc(-c2ccc(N3CCn4c(C)nc(-c5cccnc5C(C)C)c4C3)cc2)o1]

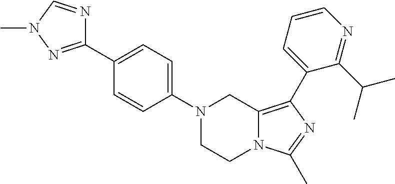 [SMILES: Cc1nc(-c2cccnc2C(C)C)c2n1CCN(c1ccc(-c3ncn(C)n3)cc1)C2]

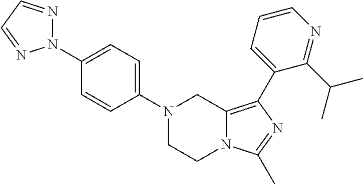 [SMILES: Cc1nc(-c2cccnc2C(C)C)c2n1CCN(c1ccc(-n3nccn3)cc1)C2]

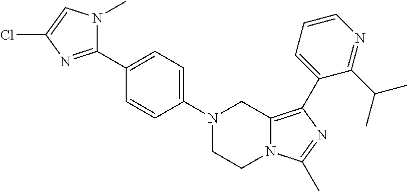 [SMILES: Cc1nc(-c2cccnc2C(C)C)c2n1CCN(c1ccc(-c3nc(Cl)cn3C)cc1)C2]

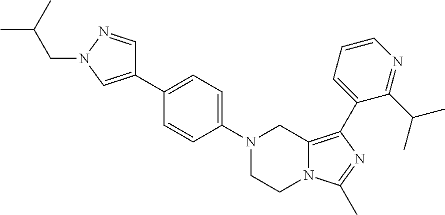 [SMILES: Cc1nc(-c2cccnc2C(C)C)c2n1CCN(c1ccc(-c3cnn(CC(C)C)c3)cc1)C2]

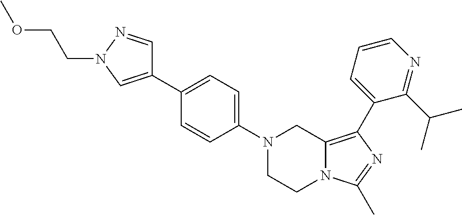 [SMILES: COCCn1cc(-c2ccc(N3CCn4c(C)nc(-c5cccnc5C(C)C)c4C3)cc2)cn1]

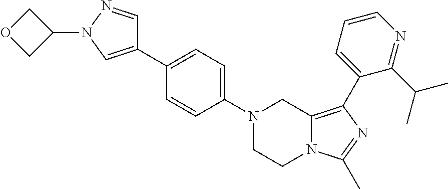 [SMILES: Cc1nc(-c2cccnc2C(C)C)c2n1CCN(c1ccc(-c3cnn(C4COC4)c3)cc1)C2]

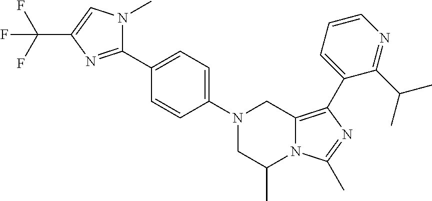 [SMILES: Cc1nc(-c2cccnc2C(C)C)c2n1C(C)CN(c1ccc(-c3nc(C(F)(F)F)cn3C)cc1)C2]

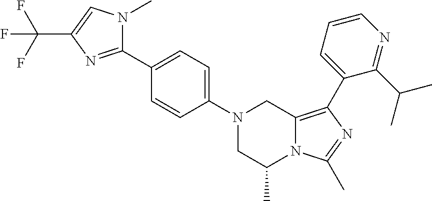 [SMILES: Cc1nc(-c2cccnc2C(C)C)c2n1[C@H](C)CN(c1ccc(-c3nc(C(F)(F)F)cn3C)cc1)C2]

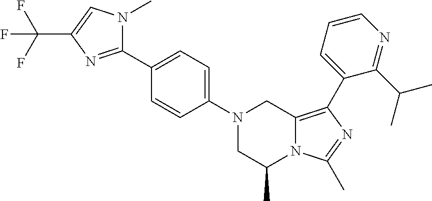 [SMILES: Cc1nc(-c2cccnc2C(C)C)c2n1[C@@H](C)CN(c1ccc(-c3nc(C(F)(F)F)cn3C)cc1)C2]

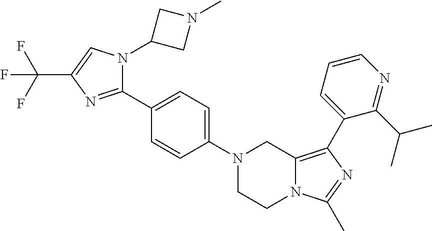 [SMILES: Cc1nc(-c2cccnc2C(C)C)c2n1CCN(c1ccc(-c3nc(C(F)(F)F)cn3C3CN(C)C3)cc1)C2]

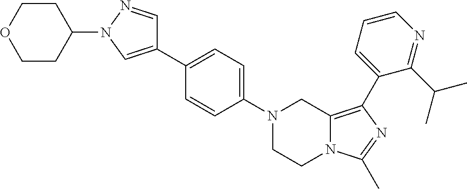 [SMILES: Cc1nc(-c2cccnc2C(C)C)c2n1CCN(c1ccc(-c3cnn(C4CCOCC4)c3)cc1)C2]

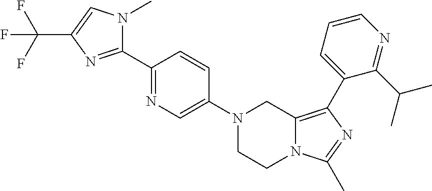 [SMILES: Cc1nc(-c2cccnc2C(C)C)c2n1CCN(c1ccc(-c3nc(C(F)(F)F)cn3C)nc1)C2]

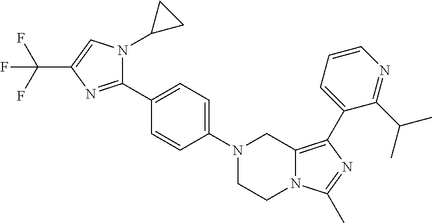 [SMILES: Cc1nc(-c2cccnc2C(C)C)c2n1CCN(c1ccc(-c3nc(C(F)(F)F)cn3C3CC3)cc1)C2]

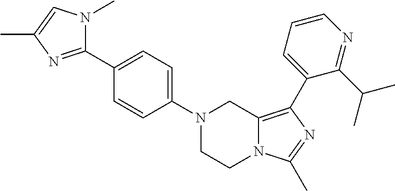 [SMILES: Cc1cn(C)c(-c2ccc(N3CCn4c(C)nc(-c5cccnc5C(C)C)c4C3)cc2)n1]

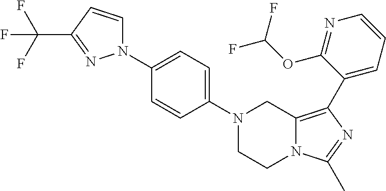 [SMILES: Cc1nc(-c2cccnc2OC(F)F)c2n1CCN(c1ccc(-n3ccc(C(F)(F)F)n3)cc1)C2]

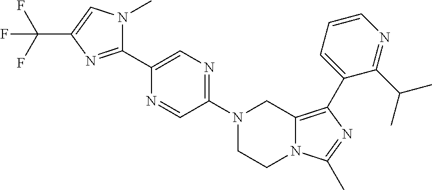 [SMILES: Cc1nc(-c2cccnc2C(C)C)c2n1CCN(c1cnc(-c3nc(C(F)(F)F)cn3C)cn1)C2]

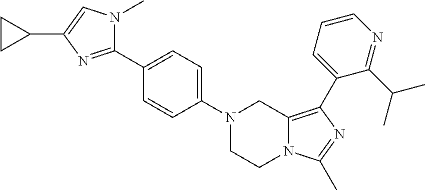 [SMILES: Cc1nc(-c2cccnc2C(C)C)c2n1CCN(c1ccc(-c3nc(C4CC4)cn3C)cc1)C2]

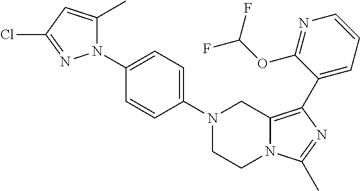 [SMILES: Cc1cc(Cl)nn1-c1ccc(N2CCn3c(C)nc(-c4cccnc4OC(F)F)c3C2)cc1]

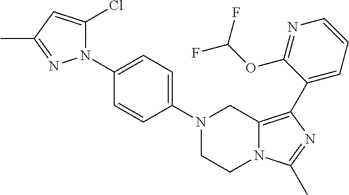 [SMILES: Cc1cc(Cl)n(-c2ccc(N3CCn4c(C)nc(-c5cccnc5OC(F)F)c4C3)cc2)n1]

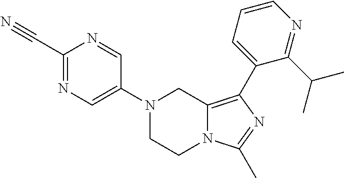 [SMILES: Cc1nc(-c2cccnc2C(C)C)c2n1CCN(c1cnc(C#N)nc1)C2]

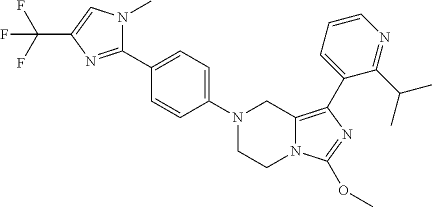 [SMILES: COc1nc(-c2cccnc2C(C)C)c2n1CCN(c1ccc(-c3nc(C(F)(F)F)cn3C)cc1)C2]

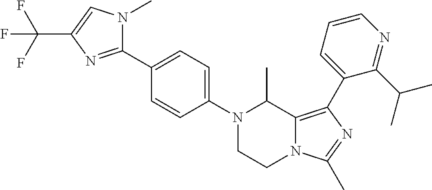 [SMILES: Cc1nc(-c2cccnc2C(C)C)c2n1CCN(c1ccc(-c3nc(C(F)(F)F)cn3C)cc1)C2C]

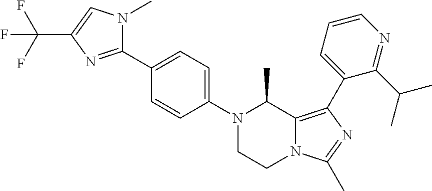 [SMILES: Cc1nc(-c2cccnc2C(C)C)c2n1CCN(c1ccc(-c3nc(C(F)(F)F)cn3C)cc1)[C@H]2C]

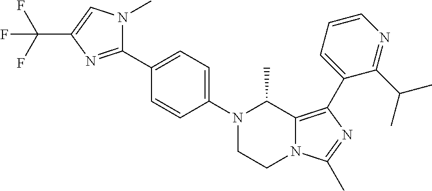 [SMILES: Cc1nc(-c2cccnc2C(C)C)c2n1CCN(c1ccc(-c3nc(C(F)(F)F)cn3C)cc1)[C@@H]2C]

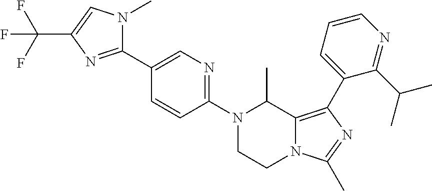 [SMILES: Cc1nc(-c2cccnc2C(C)C)c2n1CCN(c1ccc(-c3nc(C(F)(F)F)cn3C)cn1)C2C]

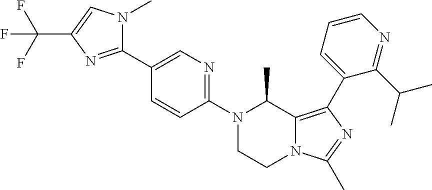 [SMILES: Cc1nc(-c2cccnc2C(C)C)c2n1CCN(c1ccc(-c3nc(C(F)(F)F)cn3C)cn1)[C@H]2C]

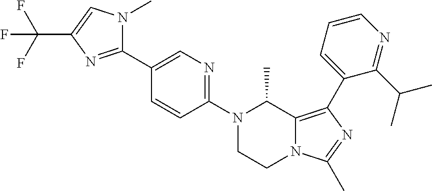 [SMILES: Cc1nc(-c2cccnc2C(C)C)c2n1CCN(c1ccc(-c3nc(C(F)(F)F)cn3C)cn1)[C@@H]2C]

In some embodiments, a compound, or a pharmaceutically acceptable salt thereof, is selected from:

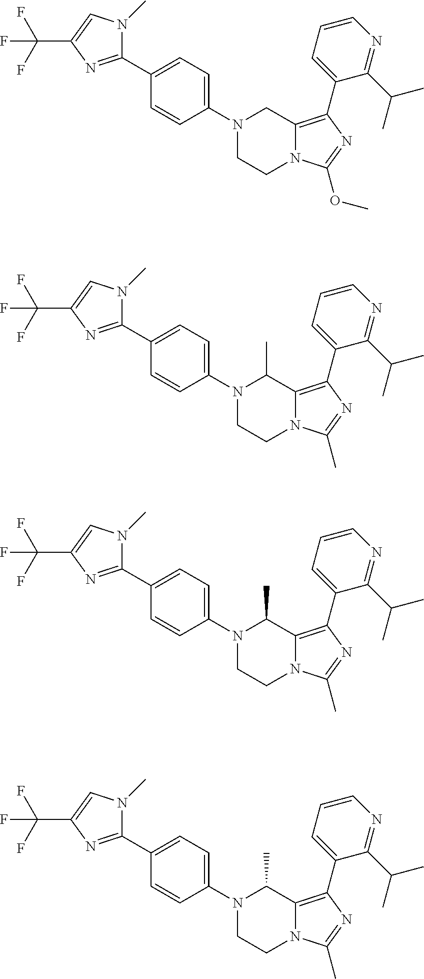 [SMILES: COc1nc(-c2cccnc2C(C)C)c2n1CCN(c1ccc(-c3nc(C(F)(F)F)cn3C)cc1)C2 ; Cc1nc(-c2cccnc2C(C)C)c2n1CCN(c1ccc(-c3nc(C(F)(F)F)cn3C)cc1)C2C ; Cc1nc(-c2cccnc2C(C)C)c2n1CCN(c1ccc(-c3nc(C(F)(F)F)cn3C)cc1)[C@H]2C ; Cc1nc(-c2cccnc2C(C)C)c2n1CCN(c1ccc(-c3nc(C(F)(F)F)cn3C)cc1)[C@@H]2C]

In some embodiments, a compound is:

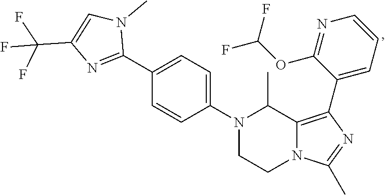 [SMILES: Cc1nc(-c2cccnc2OC(F)F)c2n1CCN(c1ccc(-c3nc(C(F)(F)F)cn3C)cc1)C2C]

or a pharmaceutically acceptable salt thereof.

In some embodiments, a compound is:

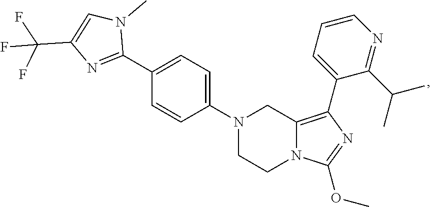 [SMILES: COc1nc(-c2cccnc2C(C)C)c2n1CCN(c1ccc(-c3nc(C(F)(F)F)cn3C)cc1)C2]

or a pharmaceutically acceptable salt thereof.

In some embodiments, a compound is:

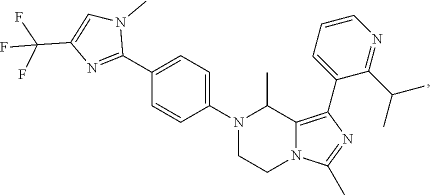 [SMILES: Cc1nc(-c2cccnc2C(C)C)c2n1CCN(c1ccc(-c3nc(C(F)(F)F)cn3C)cc1)C2C]

or a pharmaceutically acceptable salt thereof.

In some embodiments, a compound is:

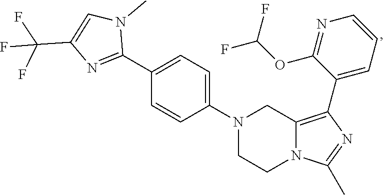 [SMILES: Cc1nc(-c2cccnc2OC(F)F)c2n1CCN(c1ccc(-c3nc(C(F)(F)F)cn3C)cc1)C2]

or a pharmaceutically acceptable salt thereof.

It will be appreciated that throughout the present disclosure, unless otherwise indicated, reference to a compound of Formula I is intended to also include IIa, IIb, IIIa, IIIb, IIIa-1, IIIb-1, IIIa-2, IIIb-2, IVa, IVb, IVa-1, IVb-1, IVa-2, IVb-2, Va, Vb, Va-1, Vb-1, Va-2, and Vb-2, and compound species of such formulas disclosed herein.

Unless otherwise stated, it will be appreciated that when “one or more” substituents are recited for a particular variable, it includes a suitable number of substituents as valency permits (e.g., one, two, three, four, or more substituents).

Unless otherwise stated, it will be appreciated that when substituents on a ring (e.g., an aryl, heteroaryl, cycloalkyl, or heterocyclyl ring) are not drawn attached to any particular position of the ring, then the substituents can be bound to any substitutable atom of said ring (e.g., a substitutable carbon atom or a substitutable nitrogen atom). For example,

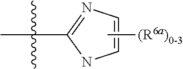

is intended to encompass at least the following exemplary embodiments:

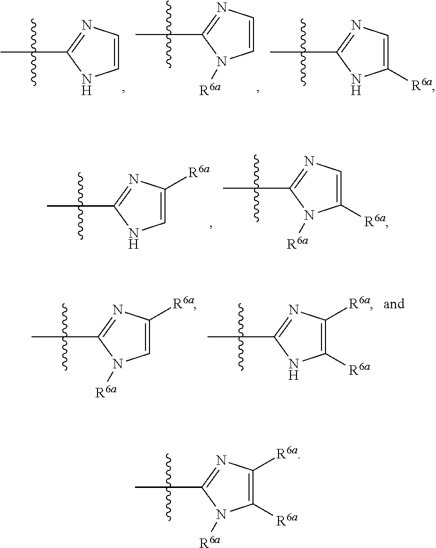

Unless otherwise stated, structures depicted herein are also meant to include all stereoisomeric (e.g., enantiomeric or diastereomeric) forms of the structure, as well as all geometric or conformational isomeric forms of the structure; for example, the R and S configurations for each stereocenter. Therefore, single stereochemical isomers, as well as enantiomeric, diastereomeric, and geometric (or conformational) mixtures of the present compounds are within the scope of the disclosure. For example, in some cases Table 1 shows one or more stereoisomers of a compound, and unless otherwise indicated, represents each stereoisomer alone and/or as a mixture. Unless otherwise stated, all tautomeric forms of the compounds of the disclosure are within the scope of the disclosure.

In some embodiments, a compound of Formula I is obtained by a process comprising a purification method in Table 4. In some embodiments, the compound is obtained by a process comprising a purification method in Table 4 and is the 1^{st} eluting isomer of the purification method. In some embodiments, the compound is obtained by a process comprising a purification method in Table 4 and is the 2^{nd} eluting isomer of the purification method. In some embodiments, the compound is obtained by a process comprising a purification method in Table 4 and is the 3^{rd} eluting isomer of the purification method. In some embodiments, the compound is obtained by a process comprising a purification method in Table 4 and is the 4^{th} eluting isomer of the purification method. In some embodiments, the compound is obtained by a process comprising a purification method in Table 4 and is the 5^{th}, 6^{th}, 7^{th}, or 8^{th} eluting isomer of the purification method.

In some embodiments, a USP1 Inhibitor is obtained by a process comprising a purification method in Table 4. In some embodiments, the USP1 Inhibitor is obtained by a process comprising a purification method in Table 4 and is the 1^{st} eluting isomer of the purification method. In some embodiments, the USP1 Inhibitor is obtained by a process comprising a purification method in Table 4 and is the 2^{nd} eluting isomer of the purification method. In some embodiments, the USP1 Inhibitor is obtained by a process comprising a purification method in Table 4 and is the 3^{rd} eluting isomer of the purification method. In some embodiments, the USP1 Inhibitor is obtained by a process comprising a purification method in Table 4 and is the 4^{th} eluting isomer of the purification method. In some embodiments, the USP1 Inhibitor is obtained by a process comprising a purification method in Table 4 and is the 5^{th}, 6^{th}, 7^{th}, or 8^{th} eluting isomer of the purification method.

Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this disclosure.

The disclosure also provides compounds of Formula I (e.g., compounds that are not USP1 Inhibitors) that are useful, for example, as analytical tools and/or control compounds in biological assays.

The compounds of Formula I may form salts which are also within the scope of this disclosure. Reference to a compound of the Formula I herein is understood to include reference to salts thereof, unless otherwise indicated. Pharmaceutically acceptable salts are well known in the art. For example, S. M. Berge, et al. describes pharmaceutically acceptable salts in detail in J. Pharmaceutical Sciences, 66: 1-19 (1977).

The disclosure also includes pharmaceutical compositions comprising one or more compounds as described herein, or a pharmaceutically acceptable salt thereof, and a pharmaceutically acceptable excipient or carrier. In some embodiments, pharmaceutical compositions reported herein can be provided in a unit dosage form (e.g., capsule, tablet, or the like). In some embodiments, pharmaceutical compositions reported herein can be provided in an oral dosage form. In some embodiments, the pharmaceutical composition is orally administered in any orally acceptable dosage form. In some embodiments, an oral dosage form of a compound of Formula I is a capsule. In some embodiments, an oral dosage form of a compound of Formula I is a tablet. In some embodiments, an oral dosage form comprises one or more fillers, disintegrants, lubricants, glidants, anti-adherents, and/or anti-statics. In some embodiments, an oral dosage form is prepared via dry blending. In some embodiments, an oral dosage form is a tablet and is prepared via dry granulation.

### Applications and Uses

The present disclosure provides a variety of uses and applications for compounds and/or compositions as described herein, for example in light of their activities and/or characteristics as described herein. In some embodiments, such uses may include therapeutic and/or diagnostic uses. Alternatively or additionally, in some embodiments such uses may include research, production, and/or other technological uses.

In some embodiments, the present disclosure provides the use of compounds of the present disclosure (e.g., a compound of Formula I, IIa, IIb, IIIa, IIIb, IIIa-1, IIIb-1, IIIa-2, IIIb-2, IVa, IVb, IVa-I, IVb-I, IVa-2, IVb-2, Va, Vb, Va-I, Vb-1, Va-2, and Vb-2) for treating, preventing, or reducing the risk of developing a disorder associated with USP1. Methods of treatment can comprise administering to a subject in need thereof a therapeutically effective amount of (i) a compound disclosed herein, or a pharmaceutically acceptable salt thereof or (ii) a pharmaceutical composition comprising a compound disclosed herein, or a pharmaceutically acceptable salt thereof, and a pharmaceutically acceptable carrier.

In some embodiments, the present disclosure provides a method of treating a disease or disorder associated with modulation of USP1 in a subject or biological sample, the method comprising administering a therapeutically effective amount of a compound disclosed herein.

In some embodiments, the present disclosure provides a method of treating a disease or disorder associated with inhibition of USP1 in a subject or biological sample, the method comprising administering a therapeutically effective amount of a compound disclosed herein.

In some embodiments, the present disclosure provides a method of inhibiting USP1 in a subject or biological sample comprising administering to said subject or biological sample a therapeutically effective amount of a compound disclosed herein.

In some embodiments, the present disclosure provides a method of treating cancer comprises administering a therapeutically effective amount of a compound disclosed herein.

### Methods of Synthesizing the Disclosed Compounds

The compounds of the present disclosure may be prepared by a variety of methods known to those of skill in the art. Suitable synthetic routes are depicted in the Schemes given below.

A general method of preparing compounds of Formula I is shown below. In some embodiments, a compound of Formula I can be prepared by coupling of Intermediate A with Intermediate B (wherein X is a suitable leaving group, including, for example, Br, OTf, Cl, or the like) in the presence of a palladium catalyst (e.g., (2-dicyclohexylphosphino-2′,6′-diisopropoxy-1,1′-biphenyl)[2-(2′-amino-1,1′-biphenyl)]palladium(II) methanesulfonate) and a base (e.g., cesium carbonate) in a solvent (e.g., dioxane) at elevated temperature.

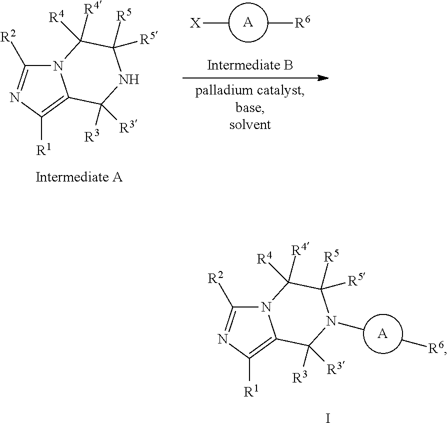

wherein Ring A, R¹, R², R³, R^{3′}, R⁴, R^{4′}, R⁵, R^{5′}, and R⁶ are defined as above and according to the classes and subclasses provided herein, and wherein X is a suitable leaving group, such as Br, OTf, Cl, or the like.

In some embodiments, the present disclosure provides a method of preparing a compound of Formula I comprising a step of contacting Intermediate A with a palladium catalyst and a base in the presence of Intermediate B. In some embodiments, the palladium catalyst is (2-dicyclohexylphosphino-2′,6′-diisopropoxy-1,1′-biphenyl)[2-(2′-amino-1,1′-biphenyl)]palladium(II) methanesulfonate. In some embodiments, the base is cesium carbonate.

### Exemplary Embodiments

The following numbered embodiments, while non-limiting, are exemplary of certain aspects of the disclosure:

1. A compound of Formula I:

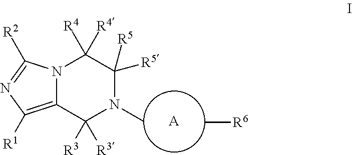

or a pharmaceutically acceptable salt thereof, wherein:
Ring A is —C₃-C₁₂ cycloalkyl, 3- to 14-membered heterocyclyl having 1 to 4 heteroatoms selected from oxygen, nitrogen, and sulfur, —C₆-C₁₀ aryl, or 5- to 14-membered heteroaryl having 1 to 4 heteroatoms selected from oxygen, nitrogen, and sulfur,
   wherein each cycloalkyl, heterocyclyl, aryl, or heteroaryl of Ring A is optionally substituted with one or more substituents selected from the group consisting of halogen, —C₁-C₆ alkyl, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, and —S(O)₂NR₂;
R¹ is —C₆-C₁₀ aryl or 5- to 14-membered heteroaryl having 1 to 4 heteroatoms selected from oxygen, nitrogen, and sulfur,
   wherein each aryl or heteoraryl of R¹ is substituted with 0, 1, 2, 3, or 4 R^{1a};
R^{1a} is halogen, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, —C₁-C₆ alkyl, —C₂-C₆ alkenyl, —C₂-C₆ alkynyl, —C₃-C₁₂ cycloalkyl, —C₄-C₁₂ cycloalkenyl, 3- to 14-membered heterocyclyl having 1 to 4 heteroatoms selected from oxygen, nitrogen, or sulfur, —C₆-C₁₀ aryl, or 5- to 14-membered heteroaryl having 1 to 4 heteroatoms selected from oxygen, nitrogen, or sulfur;
R² is —H, halogen, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, —C₁-C₆ alkyl, —C₂-C₆ alkenyl, —C₂-C₆ alkynyl, —C₃-C₁₂ cycloalkyl, —C₄-C₁₂ cycloalkenyl, 3- to 14-membered heterocyclyl having 1 to 4 heteroatoms selected from oxygen, nitrogen, or sulfur, —C₆-C₁₀ aryl, or 5- to 14-membered heteroaryl having 1 to 4 heteroatoms selected from oxygen, nitrogen, or sulfur,
   wherein each alkyl, alkenyl, alkynyl, cycloalkyl, cycloalkenyl, heterocyclyl, aryl, or heteroaryl of R² is optionally substituted with one or more halogen, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, —C₁-C₆ alkyl, —C₃-C₁₂ cycloalkyl, 3- to 14-membered heterocyclyl having 1 to 4 heteroatoms selected from oxygen, nitrogen, or sulfur, —C₆-C₁₀ aryl, or 5- to 14-membered heteroaryl having 1 to 4 heteroatoms selected from oxygen, nitrogen, or sulfur;
R³, R^{3′}, R⁴, R^{4′}, R⁵, and R^{5′} are each independently selected from the group consisting of —H, —C₁-C₆ alkyl, —C₂-C₆ alkenyl, —C₂-C₆ alkynyl, and —C₃-C₇ cycloalkyl; or each of (R³ and R^{3′}), or (R⁴ and R^{4′}), or (R⁵ and R^{5′}) can combine with the atom to which they are attached form a —C₃-C₁₂ cycloalkyl ring or 3- to 14-membered heterocyclyl ring having 1 to 4 heteroatoms selected from oxygen, nitrogen, or sulfur;
R⁶ is —H, halogen, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, —C₁-C₆ alkyl, —C₂-C₆ alkenyl, —C₂-C₆ alkynyl, —C₃-C₁₂ cycloalkyl, —C₄-C₁₂ cycloalkenyl, 3- to 14-membered heterocyclyl having 1 to 4 heteroatoms selected from oxygen, nitrogen, or sulfur, —C₆-C₁₀ aryl, or 5- to 14-membered heteroaryl having 1 to 4 heteroatoms selected from oxygen, nitrogen, or sulfur,
   wherein each alkyl, cycloalkyl, cycloalkenyl, heterocyclyl, aryl, or heteroaryl is substituted with 0, 1, 2, 3, or 4 R^{6a};
each R^{6a} is independently selected from the group consisting of halogen, oxo, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, —C₁-C₆ alkyl, —C₂-C₆ alkenyl, —C₂-C₆ alkynyl, —C₃-C₁₂ cycloalkyl, —C₄-C₁₂ cycloalkenyl, 3- to 14-membered heterocyclyl having 1 to 4 heteroatoms selected from oxygen, nitrogen, or sulfur, —C₆-C₁₀ aryl, and 5- to 14-membered heteroaryl having 1 to 4 heteroatoms selected from oxygen, nitrogen, or sulfur,
   wherein each alkyl, alkenyl, alkynyl, cycloalkyl, cycloalkenyl, heterocyclyl, aryl, or heteroaryl of R^{6a} is optionally substituted with one or more halogen, —C₁-C₆ alkyl, or —OR;
each R is independently selected from the group consisting of —H, —C₁-C₆ alkyl, —C₀-C₆ alkylene-C₆-C₁₀ aryl, —C₃-C₁₂ cycloalkyl, 3- to 14-membered heterocyclyl having 1 to 4 heteroatoms selected from oxygen, nitrogen, or sulfur, or 5- to 14-membered heteroaryl having 1 to 4 heteroatoms selected from oxygen, nitrogen, or sulfur,
   wherein each alkyl, aryl, cycloalkyl, heterocyclyl, or heteroaryl of R is substituted with 0, 1, 2, 3, or 4 R^{a};
or two R groups can combine with the atom to which they are attached to form a 5- to 6-membered heterocyclyl having 1 to 4 heteroatoms selected from oxygen, nitrogen, or sulfur, optionally substituted with —C₁-C₆ alkyl;
R^{a} is halogen, —O(C₁-C₆ alkyl), —NH(C₁-C₆ alkyl), —N(C₁-C₆ alkyl)₂, —C₃-C₆ cycloalkyl, 5- to 6-membered heterocyclyl having 1 to 4 heteroatoms selected from oxygen, nitrogen, or sulfur, or 5- to 6-membered heteroaryl having 1 to 4 heteroatoms selected from oxygen, nitrogen, or sulfur,
   wherein each cycloalkyl, heterocyclyl, or heteroaryl of R^{a} is optionally substituted with —C₁-C₆ alkyl or —C₁-C₆ alkyl substituted with one or more halogen; and
each R′ is independently selected from the group consisting of —C₁-C₆ alkyl, —C₂-C₆ alkenyl, —C₂-C₆ alkynyl, —C₃-C₁₂ cycloalkyl, —C₄-C₁₂ cycloalkenyl, 3- to 14-membered heterocyclyl having 1 to 4 heteroatoms selected from oxygen, nitrogen, or sulfur, —C₆-C₁₀ aryl, and 5- to 14-membered heteroaryl having 1 to 4 heteroatoms selected from oxygen, nitrogen, or sulfur.
   2. The compound of embodiment 1, wherein Ring A is —C₆-C₁₀ aryl or 5- to 14-membered heteroaryl having 1 to 4 heteroatoms selected from oxygen, nitrogen, and sulfur, wherein each aryl or heteroaryl of Ring A is optionally substituted with one or more substituents selected from the group consisting of halogen, —C₁-C₆ alkyl, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, and —S(O)₂NR₂.
   3. The compound of any one of the preceding embodiments, wherein Ring A is phenyl or 6-membered heteroaryl having 1 to 3 nitrogen atoms, wherein each phenyl or heteroaryl of Ring A is optionally substituted with one or more substituents selected from the group consisting of halogen, —C₁-C₆ alkyl, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, and —S(O)₂NR₂.
   4. The compound of any one of the preceding embodiments, wherein Ring A is phenyl or pyridyl.
   5. The compound of any one of the preceding embodiments, wherein Ring A is phenyl.
   6. The compound of any one of the preceding embodiments, wherein R¹ is phenyl or 6-membered heteroaryl having 1 to 3 nitrogen atoms, wherein each phenyl or heteroaryl is substituted with 0, 1, 2, 3, or 4 R^{1a}.
   7. The compound of any one of the preceding embodiments, wherein R¹ is phenyl or 6-membered heteroaryl having 1 to 3 nitrogen atoms, wherein each phenyl or heteroaryl is substituted with 1 R^{1a}.
   8. The compound of any one of the preceding embodiments, wherein R^{1a} is halogen, —OR, —NR₂, —CN, —NO₂, —SR, —C₁-C₆ alkyl, —C₃-C₆ cycloalkyl, or 3- to 6-membered heterocyclyl having 1 to 3 heteroatoms selected from oxygen, nitrogen, or sulfur, wherein R is —C₁-C₆ alkyl optionally substituted with one or more halogen.
   9. The compound of any one of the preceding embodiments, wherein R^{1a} is —OR or —C₁-C₆ alkyl.
   10. The compound of any one of the preceding embodiments, wherein R^{1a} is —OR or —C₁-C₆ alkyl, wherein R is —C₁-C₆ alkyl optionally substituted with one or more halogen.
   11. The compound of any one of the preceding embodiments, wherein R^{1a} is selected from the group consisting of isopropyl, —OCHF₂, —OCH₂CF₃, and —OCH(CH₃)₂.
   12. The compound of any one of the preceding embodiments, wherein R^{1a} is isopropyl.
   13. The compound of any one of the preceding embodiments, wherein R^{1a} is —OCHF₂.
   14. The compound of any one of the preceding embodiments, wherein R² is —H, halogen, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, —C₁-C₆ alkyl, —C₃-C₆ cycloalkyl, or 3- to 6-membered heterocyclyl having 1 to 3 heteroatoms selected from oxygen, nitrogen, or sulfur, wherein each alkyl, cycloalkyl, or heterocyclyl of R² is optionally substituted with one or more halogen, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, or —S(O)₂NR₂.
   15. The compound of any one of the preceding embodiments, wherein R² is —H, —OR, —C₁-C₆ alkyl, or —C₃-C₁₂ cycloalkyl, wherein each alkyl or cycloalkyl of R² is optionally substituted with one or more halogen.
   16. The compound of any one of the preceding embodiments, wherein R² is selected from the group consisting of —H, methyl, —CF₃, —OCH₃, and cyclopropyl.
   17. The compound of any one of the preceding embodiments, wherein R² is methyl.
   18. The compound of any one of the preceding embodiments, wherein R³, R^{3′}, R⁴, R^{4′}, R⁵, and R^{5′} are each independently selected from the group consisting of —H and —C₁-C₆ alkyl.
   19. The compound of any one of the preceding embodiments, wherein R³, R⁴, and R⁵ are each independently selected from the group consisting of —H and —C₁-C₆ alkyl.
   20. The compound of any one of the preceding embodiments, wherein R³ is methyl, and R^{3′}, R⁴, R^{4′}, R⁵, and R^{5′} are —H.
   21. The compound of any one of the preceding embodiments, wherein R⁴ is methyl, and R³, R^{3′}, R^{4′}, R⁵, and R^{5′} are —H.
   22. The compound of any one of the preceding embodiments, wherein R⁵ is methyl, and R³, R^{3′}, R⁴, R^{4′}, and R^{5′} are —H.
   23. The compound of any one of the preceding embodiments, wherein R³, R^{3′}, R⁴, R^{4′}, R⁵, and R^{5′} are each —H.
   24. The compound of any one of the preceding embodiments, wherein R⁶ is selected from the group consisting of —H, halogen, —OR, —NR₂, —NRC(O)R′, —CN, —C(O)NR₂, —C₁-C₆ alkyl, 3- to 14-membered heterocyclyl having 1 to 4 heteroatoms selected from oxygen, nitrogen, or sulfur, or 5- to 14-membered heteroaryl having 1 to 4 heteroatoms selected from oxygen, nitrogen, or sulfur, wherein each alkyl, heterocyclyl, aryl, or heteroaryl of R⁶ is substituted with 0, 1, 2, 3, or 4 R^{6a}.
   25. The compound of any one of the preceding embodiments, wherein R⁶ is 5-membered heteroaryl having 1 to 4 heteroatoms selected from oxygen, nitrogen, and sulfur, wherein the heteroaryl of R⁶ is substituted with 0, 1, 2, 3, or 4 R^{6a}.
   26. The compound of any one of the preceding embodiments, wherein R⁶ is 5-membered heteroaryl having 1 to 4 heteroatoms selected from oxygen, nitrogen, and sulfur, wherein the heteroaryl of R⁶ is substituted with 0, 1, or 2 R^{6a}.
   27. The compound of any one of the preceding embodiments, wherein R⁶ is imidazolyl substituted with 2 R^{6a}.
   28. The compound of any one of the preceding embodiments, wherein R⁶ is:

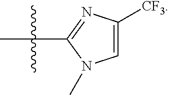 [SMILES: *c1nc(C(F)(F)F)cn1C]

29. The compound of any one of the preceding embodiments, wherein each R^{6a} is independently selected from the group consisting of halogen, —C₁-C₆ alkyl, —C₃-C₁₂ cycloalkyl, and 3- to 14-membered heterocyclyl having 1 to 4 heteroatoms selected from oxygen, nitrogen, or sulfur, wherein each alkyl, cycloalkyl, or heterocyclyl of R^{6a} is optionally substituted with one or more halogen, —C₁-C₆ alkyl, or —OR.
30. The compound of any one of the preceding embodiments, wherein each R^{6a} is independently —C₁-C₆ alkyl optionally substituted with one or more halogen.
31. The compound of any one of the preceding embodiments, wherein each R^{6a} is independently methyl or —CF₃.
32. The compound of any one of the preceding embodiments, wherein each R is independently selected from the group consisting of —H, —C₁-C₆ alkyl, —C₀-C₆ alkylene-C₆-C₁₀ aryl, and —C₃-C₁₂ cycloalkyl, wherein each alkyl, aryl, or cycloalkyl of R is substituted with 0, 1, 2, 3, or 4 R^{a}, or two R groups can combine with the atom to which they are attached to form a 5- to 6-membered heterocyclyl having 1 to 4 heteroatoms selected from oxygen, nitrogen, or sulfur, optionally substituted with —C₁-C₆ alkyl.
33. The compound of any one of the preceding embodiments, wherein each R is independently selected from the group consisting of —H and —C₁-C₆ alkyl, wherein each alkyl of R is substituted with 0, 1, 2, 3, or 4 R^{a}.
34. The compound of any one of the preceding embodiments, wherein each R^{a} is independently selected from the group consisting of halogen, 5- to 6-membered heterocyclyl having 1 to 4 heteroatoms selected from oxygen, nitrogen, or sulfur, or 5- to 6-membered heteroaryl having 1 to 4 heteroatoms selected from oxygen, nitrogen, or sulfur, wherein each heterocyclyl or heteroaryl of R^{a} is optionally substituted with —C₁-C₆ alkyl substituted with one or more halogen.
35. The compound of any one of the preceding embodiments, wherein each R′ is independently selected from the group consisting of —C₁-C₆ alkyl and —C₃-C₁₂ cycloalkyl.
36. The compound of any one of the preceding embodiments, wherein each R′ is cyclopropyl.
37. The compound of any one of the preceding embodiments, wherein the compound is of Formula IIa or Formula IIb:

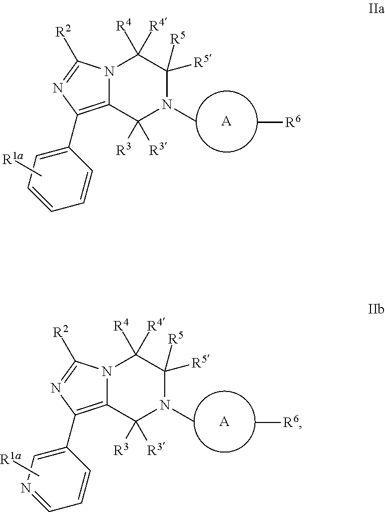

or a pharmaceutically acceptable salt thereof.
38. The compound of any one of the preceding embodiments, wherein the compound is of Formula IIIa or IIIb:

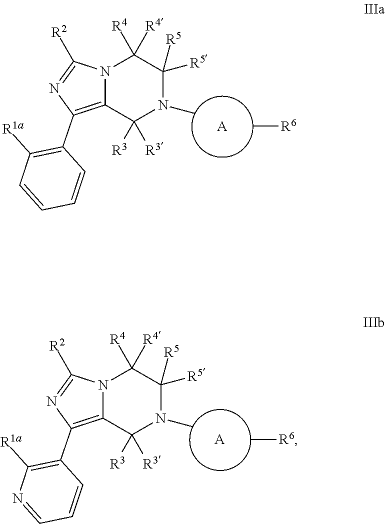

or a pharmaceutically acceptable salt thereof.
39. The compound of any one of the preceding embodiments, wherein the compound is of Formula IIIa-1 or IIIb-1:

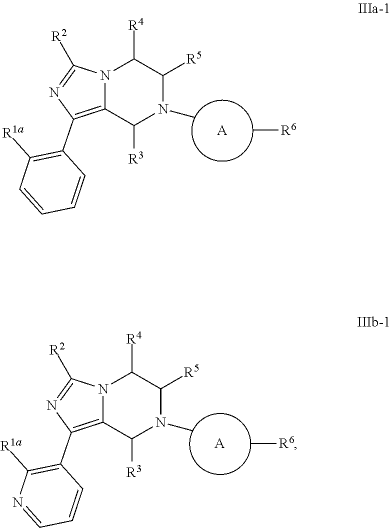

or a pharmaceutically acceptable salt thereof.
40. The compound of any one of the preceding embodiments, wherein the compound is of Formula IIIa-2 or Formula IIIb-2:

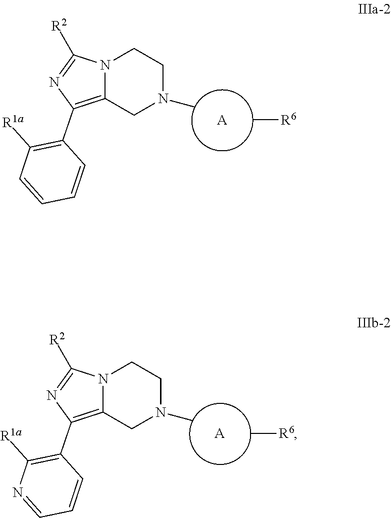

or a pharmaceutically acceptable salt thereof.
41. The compound of any one of the preceding embodiments, wherein the compound is of Formula IVa or Formula IVb:

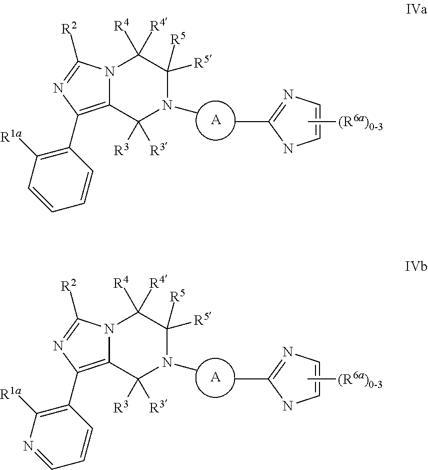

or a pharmaceutically acceptable salt thereof.
42. The compound of any one of the preceding embodiments, wherein the compound is of Formula IVa-1 or Formula IVb-1:

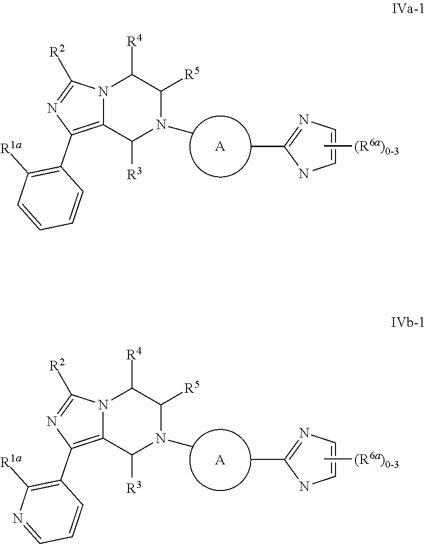

or a pharmaceutically acceptable salt thereof.
43. The compound of any one of the preceding embodiments, wherein the compound is of Formula IVa-2 or Formula IVb-2:

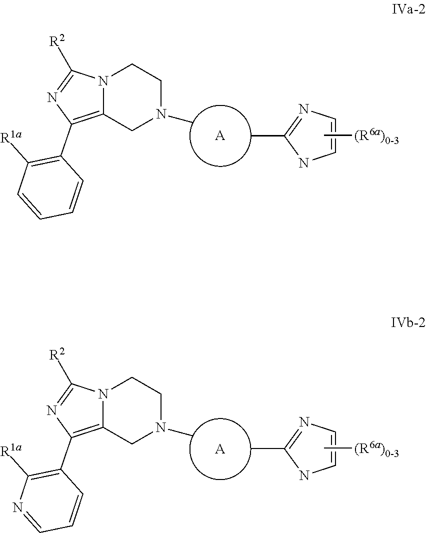

or a pharmaceutically acceptable salt thereof.
44. The compound of any one of the preceding embodiments, wherein the compound is of Formula Va or Formula Vb:

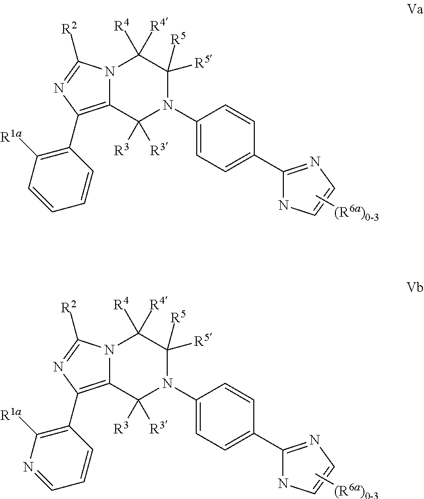

or a pharmaceutically acceptable salt thereof.
45. The compound of any one of the preceding embodiments, wherein the compound is of Formula Va-1 or Formula Vb-1:

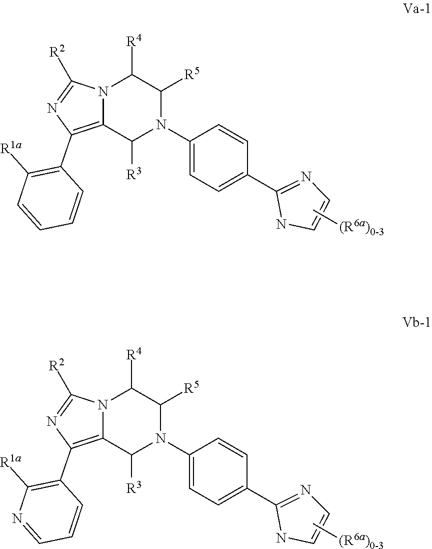

or a pharmaceutically acceptable salt thereof.
46. The compound of any one of the preceding embodiments, wherein the compound is of Formula Va-2 or Formula Vb-2:

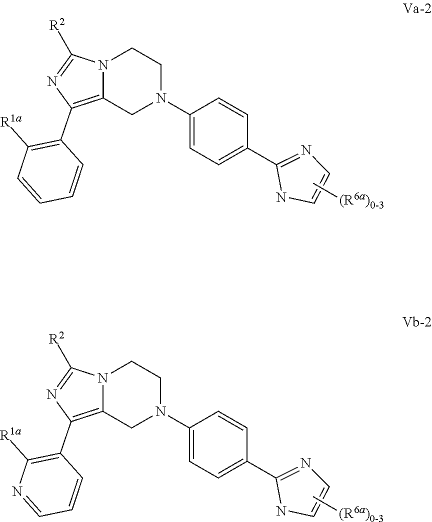

or a pharmaceutically acceptable salt thereof.
47. The compound of any one of the preceding embodiments, wherein:
Ring A is —C₆-C₁₀ aryl or 5- to 14-membered heteroaryl having 1 to 4 heteroatoms selected from oxygen, nitrogen, and sulfur,
   wherein each aryl or heteroaryl of Ring A is optionally substituted with one or more halogen;
R^{1a} is —OR or —C₁-C₆ alkyl;
R² is —H, —OR, —C₁-C₆ alkyl, or —C₃-C₁₂ cycloalkyl,
   wherein each alkyl or cycloalkyl of R² is optionally substituted with one or more halogen;
R³, R⁴, and R⁵ are each independently selected from the group consisting of —H or —C₁-C₆ alkyl;
R⁶ is —OR, —NRC(O)R′, —C(O)NR₂, —C₁-C₆ alkyl, 3- to 14-membered heterocyclyl having 1 to 4 heteroatoms selected from oxygen, nitrogen, or sulfur, or 5- to 14-membered heteroaryl having 1 to 4 heteroatoms selected from oxygen, nitrogen, or sulfur,
   wherein each alkyl, heterocyclyl, or heteroaryl of R⁶ is substituted with 0, 1, 2, 3, or 4 R^{6a};
each R^{6a} is independently selected from the group consisting of halogen, oxo, —OR, —C₁-C₆ alkyl, —C₃-C₁₂ cycloalkyl, or 3- to 14-membered heterocyclyl having 1 to 4 heteroatoms selected from oxygen, nitrogen, or sulfur,
   wherein each alkyl, cycloalkyl, or heterocyclyl of R^{6a} is optionally substituted with one or more halogen, —C₁-C₆ alkyl, or —OR;
each R is independently selected from the group consisting of —H, —C₁-C₆ alkyl or —C₀-C₆ alkylene-C₆-C₁₀ aryl,
   wherein each alkyl or aryl of R is substituted with 0, 1, 2, 3, or 4 R^{a};
or two R groups can combine with the atom to which they are attached to form a 5- to 6-membered heterocyclyl having 1 to 4 heteroatoms selected from oxygen, nitrogen, or sulfur, optionally substituted with —C₁-C₆ alkyl;
R^{a} is halogen, 5- to 6-membered heterocyclyl having 1 to 4 heteroatoms selected from oxygen, nitrogen, or sulfur, or 5- to 6-membered heteroaryl having 1 to 4 heteroatoms selected from oxygen, nitrogen, or sulfur,
   wherein each heteroaryl of R^{a} is optionally substituted with —C₁-C₆ alkyl substituted with one or more halogen; and
each R′ is independently —C₃-C₁₂ cycloalkyl.
   48. A compound selected from Table 1.
   49. A compound of any one of the preceding embodiments, wherein the compound is a USP1 Inhibitor having an IC₅₀ value ≤10 μM in the assay of Example 1.
   50. A compound of any one of the preceding embodiments, wherein the compound is a USP1 Inhibitor having an IC₅₀ value ≤1 μM in the assay of Example 1.
   51. A pharmaceutical composition, comprising a compound of any one of the preceding embodiments, or a pharmaceutically acceptable salt thereof, and a pharmaceutically acceptable excipient.

### Examples

The present teachings including descriptions provided in the Examples are not intended to limit the scope of any claim. Unless specifically presented in the past tense, inclusion in the Examples is not intended to imply that the experiments were actually performed. The following non-limiting examples are provided to further illustrate the present teachings. Those of skill in the art, in light of the present disclosure, will appreciate that many changes can be made in the specific embodiments that are disclosed and still obtain a like or similar result without departing from the spirit and scope of the present teachings.

### Analytical Methods, Materials, and Instrumentation

Unless otherwise noted, reagents and solvents were used as received from commercial suppliers. Unless otherwise noted, reactions were conducted under an inert atmosphere of nitrogen. NMR instruments: Bruker BBFO ASCEND™400 AVANCE III 400 MHz and Bruker BBFO ULTRASHIELD™300 AVANCE III 300 MHz. Internal standard: Tetramethylsilane (TMS). MassSpec instruments and ionization method: Shimadzu LC-2020, electrospray ionization (ESI). Chromatography instruments: Reverse phase chromatography: Agela TechnologiesMP200; preparatory HPLC (Prep-HPLC): Waters; supercritical fluid chromatography (SFC): Shimadzu.

### Abbreviations

Boc₂O di-tert-butyl dicarbonate
Bn benzyl
bpy 2,2′-bipyridyl
δ chemical shift
DCM dichloromethane
DIEA N,N-diisopropylethylamine
DMAP 4-(dimethylamino)pyridine
DMF N,N-dimethylformamide
DMP Dess-Martin periodinane
DMSO dimethylsulfoxide
dppf 1,1′-bis(diphenylphosphino)ferrocene
EA ethyl acetate
ee enantiomeric excess
equiv equivalents
h hour
H NMR proton nuclear magnetic resonance
HATU 2-(3H-[1,2,3]triazolo[4,5-b]pyridin-3-yl)-1,1,3,3-tetramethylisouronium hexafluorophosphate
HPLC high performance liquid chromatography
Hz Hertz
LCMS liquid chromatography/mass spectrometry
MeOH methanol
min minutes
MS mass spectrometry
NBS N-bromosuccinimide
Pd(dppf)Cl₂ [1,1′-Bis(diphenylphosphino)ferrocene]dichloropalladium(II) chloride
rt room temperature
RuPhos-Pd-G3 (2-dicyclohexylphosphino-2′,6′-diisopropoxy-1,1′-biphenyl)[2-(2′-amino-1,1′-biphenyl)]palladium(II) methanesulfonate
TEA triethylamine
TFA trifluoroacetic acid Tf₂O trifluoromethanesulfonic anhydride
THE tetrahydrofuran
TLC thin layer chromatography
tol toluene
TsCl p-toluenesulfonyl chloride
XPhos-Pd-G3 methanesulfonato(2-dicyclohexylphosphino-2′,4′,6′-triisopropyl-1,1′-biphenyl)(2′-amino-1,1′-biphenyl-2-yl)palladium(II)

### Synthesis of Intermediates

### Intermediate A-1. 1-(2-isopropylpyridin-3-yl)-3-methyl-5,6,7,8-tetrahydroimidazo[1,5-a]pyrazine

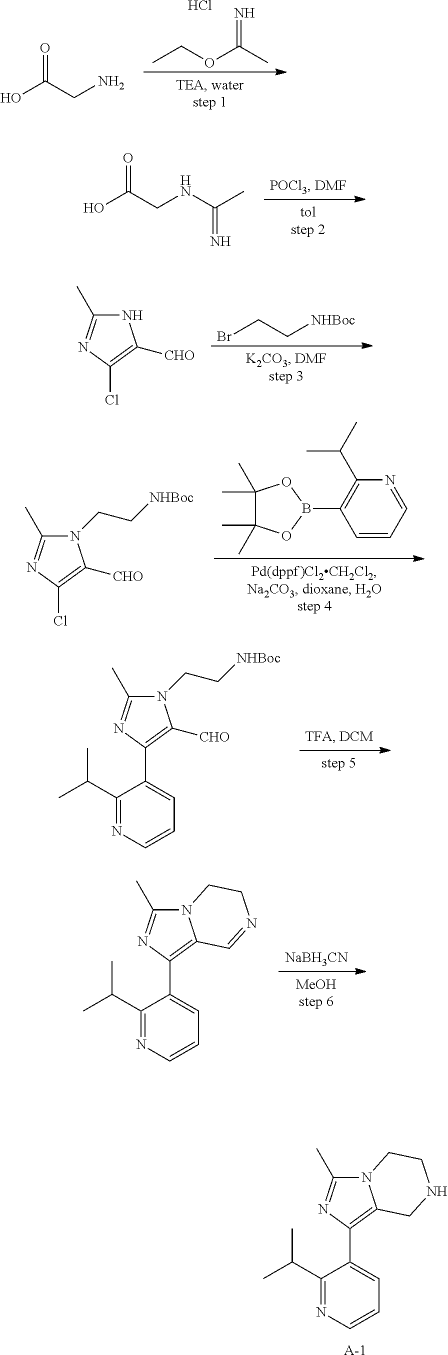

### Step 1. (1-iminoethyl)glycine

A solution of 2-aminoacetic acid (100 g, 1.33 mol, 0.65 equiv) in water (1250 mL) was treated with TEA (150 mL, 1.08 mol, 0.53 equiv) to bring the pH to 10. The solution was treated by portionwise addition of ethyl acetimidate hydrochloride (250 g, 2.02 mol, 1.00 equiv) at rt with concurrent dropwise addition of TEA to maintain pH of the reaction mixture at 9.5-10. The resulting solution was stirred for 6 h at rt, then was concentrated under vacuum and then treated with ethanol (1600 mL). The solids were collected by filtration and dried under vacuum to afford 136 g (57%) of (1-iminoethyl)glycine as a white solid. LCMS (ESI, m z) 117.0 [M+H]⁺.

### Step 2. 4-chloro-2-methyl-1H-imidazole-5-carbaldehyde

A mixture of (1-iminoethyl)glycine (50 g, 421.98 mmol, 1.00 equiv), toluene (500 mL) and POCl₃ (184 g, 1.20 mol, 2.84 equiv) was treated by dropwise addition of DMF (95 g, 1.30 mol, 3.00 equiv) with stirring at 80° C. The resulting solution was stirred for 2 h at 100° C., then cooled to rt, the reaction mixture was poured into ice/water (600 mL) and then basified to pH of 2 with sodium hydroxide solution (4 N). The resulting solution was extracted with ethyl acetate (500 mL×2). The organic layers were combined, dried over anhydrous sodium sulfate, filtered and concentrated under vacuum. The residue was purified by silica gel chromatography (eluting with 20/80 to 80/20 ethyl acetate/petroleum ether) to afford 18 g (28%) of 4-chloro-2-methyl-1H-imidazole-5-carbaldehyde as an orange solid. LCMS (ESI, m z) 145.0 [M+H]⁺.

### Step 3. tert-butyl (2-(4-chloro-5-formyl-2-methyl-1H-imidazol-1-yl)ethyl)carbamate

A mixture of 4-chloro-2-methyl-1H-imidazole-5-carbaldehyde (10 g, 69.18 mmol, 1.00 equiv), tert-butyl N-(2-bromoethyl)carbamate (15.5 g, 69.17 mmol, 1.00 equiv), potassium carbonate (19 g, 137.48 mmol, 2.00 equiv) and DMF (250 mL) was stirred for 2 h at 50° C. then cooled to rt. The reaction mixture was treated with water (800 mL) and was extracted with ethyl acetate (500 mL×2). The organic layers were combined, dried over anhydrous sodium sulfate, filtered and concentrated under vacuum. The residue was purified by silica gel chromatography (eluting with 0/100 to 70/30 ethyl acetate/petroleum ether) to afford 10 g (50%) of tert-butyl (2-(4-chloro-5-formyl-2-methyl-1H-imidazol-1-yl)ethyl)carbamate as an orange solid. LCMS (ESI, m/z) 288.1 [M+H]⁺.

### Step 4. tert-butyl (2-(5-formyl-4-(2-isopropylpyridin-3-yl)-2-methyl-11H-imidazol-1-yl)ethyl)carbamate

A mixture of tert-butyl (2-(4-chloro-5-formyl-2-methyl-1H-imidazol-1-yl)ethyl)carbamate (2 g, 6.95 mmol, 1.00 equiv), 2-(propan-2-yl)-3-(tetramethyl-1,3,2-dioxaborolan-2-yl)pyridine (1.89 g, 7.65 mmol, 1.20 equiv), Pd(dppf)Cl₂—CH₂Cl₂ (567 mg, 0.69 mmol, 0.10 equiv), sodium carbonate (1.47 g, 13.87 mmol, 2.00 equiv), 1,4-dioxane (50 mL) and water (10 mL) was stirred overnight at 100° C. in an oil bath. After cooling to rt, the reaction mixture was filtered and concentrated under vacuum. The residue was purified by silica gel chromatography (eluting with 30/70 to 70/30 ethyl acetate/petroleum ether) to afford 1.3 g (50%) of tert-butyl (2-(5-formyl-4-(2-isopropylpyridin-3-yl)-2-methyl-1H-imidazol-1-yl)ethyl)carbamate as a yellow solid. LCMS (ESI, m z) 373.0 [M+H]⁺.

### Step 5. 1-(2-isopropylpyridin-3-yl)-3-methyl-5,6-dihydroimidazo[1,5-a]pyrazine

A mixture of tert-butyl (2-(5-formyl-4-(2-isopropylpyridin-3-yl)-2-methyl-1H-imidazol-1-yl)ethyl)carbamate (1.3 g, 1.00 equiv, 3.5 mmol), DCM (13 mL) and TFA (3 mL) was stirred for 30 min at rt. The reaction mixture was concentrated under vacuum to afford 1 g (crude) of 1-(2-isopropylpyridin-3-yl)-3-methyl-5,6-dihydroimidazo[1,5-a]pyrazine as a red oil. LCMS (ESI, m z) 254.7 [M+H]⁺.

### Step 6. 1-(2-isopropylpyridin-3-yl)-3-methyl-5,6,7,8-tetrahydroimidazo[1,5-a]pyrazine

A mixture of 1-(2-isopropylpyridin-3-yl)-3-methyl-5,6-dihydroimidazo[1,5-a]pyrazine (950 mg, 3.49 mmol, 1.00 equiv) and MeOH (10 mL) was treated by portionwise addition of NaBH₃CN (440 mg, 7.00 mmol, 2.00 equiv) with stirring at 0° C. The resulting solution was stirred for 1 h at 0° C., then was treated with saturated NaHCO₃ solution (3 mL) and then extracted with ethyl acetate (3×10 mL). The organic layers were combined, dried over anhydrous sodium sulfate, filtered, and concentrated under vacuum. The residue was purified by silica gel chromatography (eluting with 0/100 to 10/90 MeOH/DCM) to afford 233 mg (26%) of 1-(2-isopropylpyridin-3-yl)-3-methyl-5,6,7,8-tetrahydroimidazo[1,5-a]pyrazine as an orange solid. LCMS (ESI, m z) 257.1 [M+H]⁺.

The following intermediates were prepared using the procedure for Intermediate A-1 with the appropriate boronic ester or boronic acid used in step 4:

TABLE 2

| Intermediate | Name | Structure | LCMS (ESI, m/z) |
|---|---|---|---|
| A-3 | 1-(2-isopropylphenyl)-3- methyl-5,6,7,8- tetrahydroimidazo[1,5- a]pyrazine | 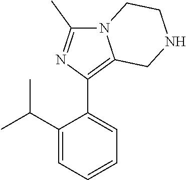 [SMILES: Cc1nc(-c2ccccc2C(C)C)c2n1CCNC2] | 256.2 [M + H]⁺ |
| A-4 | 3-methyl-1-(2-(2,2,2- trifluoroethoxy)pyridin-3-yl)- 5,6,7,8-tetrahydroimidazo[1,5- a]pyrazine | 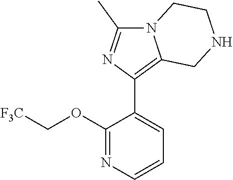 [SMILES: Cc1nc(-c2cccnc2OCC(F)(F)F)c2n1CCNC2] | 313.3 [M + H]⁺ |

### Intermediate A-2. 1-(2-(difluoromethoxy)pyridin-3-yl)-3-methyl-5,6,7,8-tetrahydroimidazo[1,5-a]pyrazine

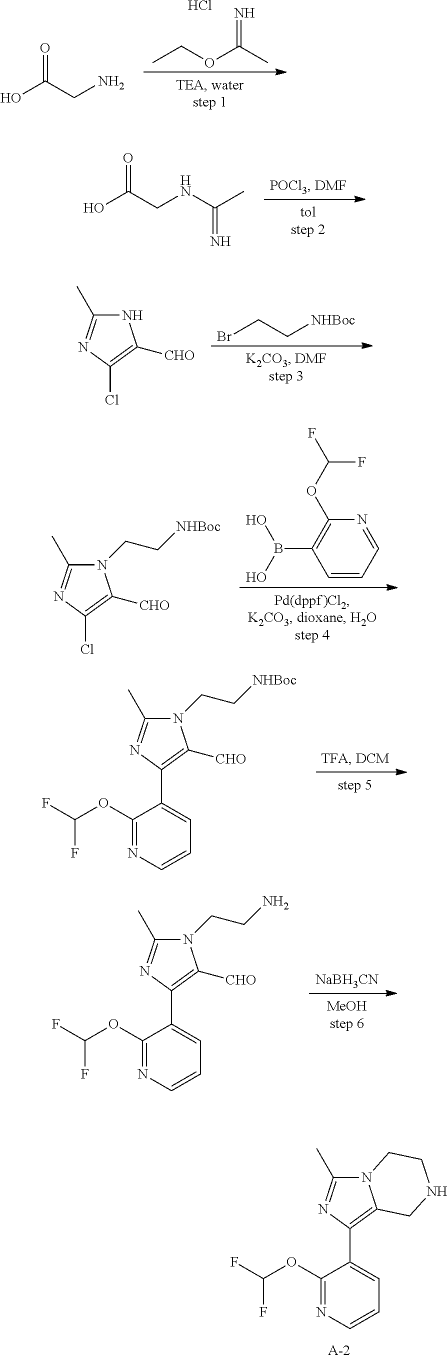

### Step 1. (1-iminoethyl)glycine

A solution of 2-aminoacetic acid (100 g, 1.33 mol, 0.65 equiv) in water (1250 mL) was treated with TEA (150 mL, 1.08 mol, 0.53 equiv) to bring the pH to 10. The solution was treated by portionwise addition of ethyl acetimidate hydrochloride (250 g, 2.02 mol, 1.00 equiv) at 0° C. The mixture was basified to pH 10 with TEA at 0° C. The resulting mixture was stirred for 6 h at rt, then was concentrated under vacuum and then treated with ethanol (1600 mL). The solids were collected by filtration and dried under vacuum to afford 136 g (57%) of (1-iminoethyl)glycine as a white solid. LCMS (ESI, m z) 117.0 [M+H]⁺.

### Step 2. 4-chloro-2-methyl-1H-imidazole-5-carbaldehyde

A mixture of (1-iminoethyl)glycine (50 g, 422 mmol, 1.00 equiv), toluene (500 mL) and POCl₃ (184 g, 1.20 mol, 2.84 equiv) was treated by dropwise addition of DMF (95 g, 1.30 mol, 3.00 equiv) with stirring at 80° C. The resulting mixture was stirred for 2 h at 100° C., then cooled to rt and concentrated under vacuum. The residue was diluted with ice/water (600 mL) and then basified to pH 2 with sodium hydroxide solution (sat., aq.). The resulting mixture was extracted with ethyl acetate (500 mL×2). The organic layers were combined, dried over anhydrous sodium sulfate, filtered and concentrated under vacuum. The residue was purified by silica gel chromatography (eluting with 1:4 ethyl acetate/petroleum ether) to afford 18 g (28%) of 4-chloro-2-methyl-1H-imidazole-5-carbaldehyde as an orange solid. LCMS (ESI, m z) 145, 147 [M+H]⁺.

### Step 3. tert-butyl (2-(4-chloro-5-formyl-2-methyl-1H-imidazol-1-yl)ethyl)carbamate

A mixture of 4-chloro-2-methyl-1H-imidazole-5-carbaldehyde (8 g, 55.3 mmol, 1.00 equiv), tert-butyl N-(2-bromoethyl)carbamate (12.4 g, 55.3 mmol, 1.00 equiv), potassium carbonate (15.3 g, 111 mmol, 2.00 equiv) and DMF (100 mL) was stirred overnight at 50° C. then cooled to rt. The reaction mixture was treated with water (200 mL) and was extracted with ethyl acetate (100 mL×3). The organic layers were combined, washed with brine (100 mL×2) dried over anhydrous sodium sulfate, filtered and concentrated under vacuum. The residue was purified by silica gel chromatography (eluting with 1:1 ethyl acetate/petroleum ether) to afford 4.40 g (28%) of tert-butyl (2-(4-chloro-5-formyl-2-methyl-1H-imidazol-1-yl)ethyl)carbamate as a light yellow solid. LCMS (ESI, m z) 288.1 [M+H]⁺.

### Step 4. tert-butyl N-(2-{4-[2-(difluoromethoxy)pyridin-3-yl]-5-formyl-2-methyl-1H-imidazol-1-yl}ethyl)carbamate

A mixture of tert-butyl (2-(4-chloro-5-formyl-2-methyl-1H-imidazol-1-yl)ethyl)carbamate (690 mg, 2.28 mmol), (2-(difluoromethoxy)pyridin-3-yl)boronic acid (500 mg, 2.64 mmol), Pd(dppf)Cl₂.CH₂Cl₂ (196 mg, 0.24 mmol), potassium carbonate (663 mg, 4.75 mmol) in dioxane (20 mL) and water (3 mL) was stirred overnight at 100° C. The reaction mixture was cooled to room temperature and filtered through a pad of Celite. The filtrate was concentrated under vacuum. The residue was purified by silica gel column chromatography, (eluting with 1:1 ethyl acetate/petroleum ether) to afford tert-butyl N-(2-{4-[2-(difluoromethoxy)pyridin-3-yl]-5-formyl-2-methyl-1H-imidazol-1-yl}ethyl)carbamate (550 mg, 58%) as a yellow solid. LCMS (ES, m z): 397 [M+H]⁺.

### Step 5. 1-(2-aminoethyl)-4-[2-(difluoromethoxy)pyridin-3-yl]-2-methyl-1H-imidazole-5-carbaldehyde (TFA salt)

To a stirred mixture of tert-butyl N-(2-{4-[2-(difluoromethoxy)pyridin-3-yl]-5-formyl-2-methyl-1H-imidazol-1-yl}ethyl)carbamate (550 mg, 1.39 mmol) in dichloromethane (10 mL) was dropwise added trifluoroacetic acid (3 mL). The resulting mixture was stirred for 1 h at 25° C. The mixture was concentrated under vacuum to afford 1-(2-aminoethyl)-4-[2-(difluoromethoxy)pyridin-3-yl]-2-methyl-1H-imidazole-5-carbaldehyde (TFA salt) (580 mg, 97%) as red oil. LCMS (ES, m z): 297 [M-TFA+H]⁺.

### Step 6. 2-(difluoromethoxy)-3-{3-methyl-5H,6H,7H,8H-imidazo[1,5-a]pyrazin-1-yl}pyridine

To a stirred mixture of 1-(2-aminoethyl)-4-[2-(difluoromethoxy)pyridin-3-yl]-2-methyl-1H-imidazole-5-carbaldehyde (TFA salt) (400 mg, 0.97 mmol) in methanol (10 mL) was added NaBH₃CN (169 mg, 2.69 mmol) in portions at 0° C. The resulting mixture was stirred for 1 h at 25° C. The mixture was diluted with sat. NaHCO₃ (20 mL) and extracted with DCM (3×20 mL). The combined organic layers were dried over anhydrous sodium sulfate, filtered, and concentrated under vacuum. The residue was purified by preparative TLC (MeOH/DCM=1/10) to afford 2-(difluoromethoxy)-3-{3-methyl-5H,6H,7H,8H-imidazo[1,5-a]pyrazin-1-yl}pyridine (200 mg, 73%) as a white solid. LCMS (ES, m z): 281 [M+H]⁺.

### Intermediate A-5. 1-(2-isopropylpyridin-3-yl)-3,8-dimethyl-5,6,7,8-tetrahydroimidazo[1,5-a]pyrazine

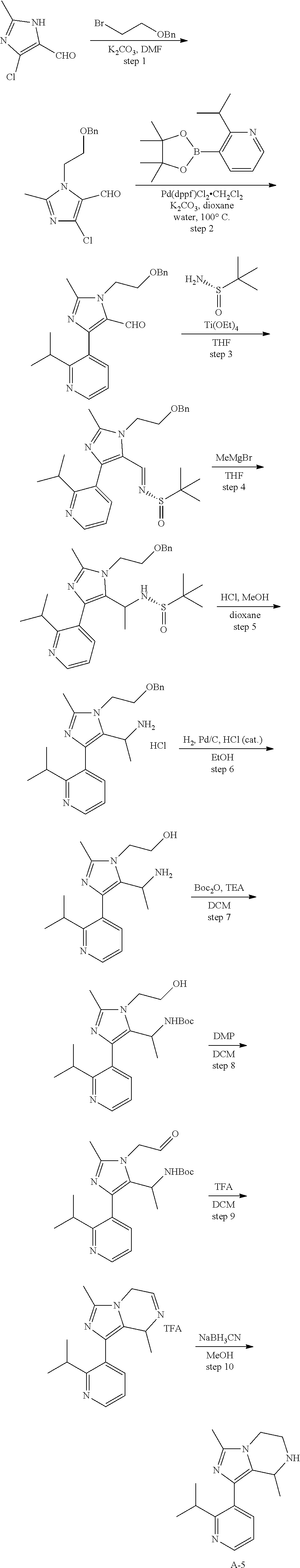

### Step 1. 1-[2-(benzyloxy)ethyl]-4-chloro-2-methyl-1H-imidazole-5-carbaldehyde

A mixture of 4-chloro-2-methyl-1H-imidazole-5-carbaldehyde (10.0 g, 69.2 mmol), [(2-bromoethoxy)methyl]benzene (22.0 g, 102 mmol) and potassium carbonate (19.0 g, 137 mmol) in DMF (200 mL) was stirred for 4 h at 50° C. After cooling to rt, the reaction mixture was poured into water (500 mL) and then was extracted with ethyl acetate (2×500 mL). The combined organic layers were dried over anhydrous sodium sulfate, filtered, and concentrated under vacuum. The resulting crude product was purified by silica gel chromatography (eluting with 0:100 to 50:50 ethyl acetate/petroleum ether) to afford 1-[2-(benzyloxy)ethyl]-4-chloro-2-methyl-1H-imidazole-5-carbaldehyde as yellow oil (10.0 g, 52%). LCMS (ESI, m z) 279, 281 [M+H]⁺.

### Step 2. 1-[2-(benzyloxy)ethyl]-2-methyl-4-[2-(propan-2-yl)pyridin-3-yl]-1H-imidazole-5-carbaldehyde

A mixture of 1-[2-(benzyloxy)ethyl]-4-chloro-2-methyl-1H-imidazole-5-carbaldehyde (2.40 g, 8.01 mmol), 2-(propan-2-yl)-3-(tetramethyl-1,3,2-dioxaborolan-2-yl)pyridine (2.55 g, 10.3 mmol), Pd(dppf)Cl₂—CH₂Cl₂ (703 mg, 0.86 mmol), water (5 mL) and potassium carbonate (2.38 g, 17.2 mmol) in 1,4-dioxane (20 mL) was stirred for 18 h at 100° C. and then cooled to rt. The reaction mixture was poured into water (100 mL) and then extracted with ethyl acetate (3×200 mL). The combined organic layers were dried over anhydrous sodium sulfate, filtered, and concentrated under vacuum. The resulting crude product was purified by silica gel chromatography (eluting with 0:100 to 50:50 ethyl acetate/petroleum ether) to afford 1-[2-(benzyloxy)ethyl]-2-methyl-4-[2-(propan-2-yl)pyridin-3-yl]-1H-imidazole-5-carbaldehyde as yellow oil (2.50 g, 86%). LCMS (ESI, m z) 364 [M+H]⁺.

### Step 3. (S)—N-[(1E)-{1-[2-(benzyloxy)ethyl]-2-methyl-4-[2-(propan-2-yl)pyridin-3-yl]-1H-imidazol-5-yl}methylidene]-2-methylpropane-2-sulfinamide

A mixture of 1-[2-(benzyloxy)ethyl]-2-methyl-4-[2-(propan-2-yl)pyridin-3-yl]-1H-imidazole-5-carbaldehyde (2.00 g, 5.50 mmol), (S)-2-methylpropane-2-sulfinamide (800 mg, 6.60 mmol) and ethyl titanate (2.30 mL, 11.0 mmol) in THE (30 mL) was stirred for 5 days at rt. The reaction was then quenched by the addition of water (1 mL). The resulting mixture was filtered through a Celite pad and the filtrate concentrated under vacuum. The residue was purified by silica gel chromatography (eluting with 0:100 to 30:70 ethyl acetate/petroleum ether) to afford (S)—N-[(1E)-[1-[2-(benzyloxy)ethyl]-2-methyl-4-[2-(propan-2-yl)pyridin-3-yl]-1H-imidazol-5-yl]methylidene]-2-methylpropane-2-sulfinamide as yellow oil (1.90 g, 74%). Note: stereochemistry of product arbitrarily assigned. LCMS (ESI, m z) 467 [M+H]⁺.

### Step 4. N-[1-{1-[2-(benzyloxy)ethyl]-2-methyl-4-[2-(propan-2-yl)pyridin-3-yl]-1H-imidazol-5-yl}ethyl]-2-methylpropane-2-sulfinamide

A mixture of (S)—N-[(1E)-[1-[2-(benzyloxy)ethyl]-2-methyl-4-[2-(propan-2-yl)pyridin-3-yl]-1H-imidazol-5-yl]methylidene]-2-methylpropane-2-sulfinamide (2.00 g, 4.29 mmol) in THE (40 mL) at −78° C. was treated methylmagnesium bromide (4.28 mL, 3 M in THF). The mixture was warmed to 0° C., stirred for 5 h, and was then poured into water (25 mL). The resulting mixture was extracted with ethyl acetate (2×250 mL). The combined organic layers were dried over anhydrous sodium sulfate, filtered, and concentrated under vacuum. The resulting crude product was purified by C₁₈ reversed phase chromatography (eluting with 20% to 45% MeCN/water (0.05% NH₄HCO₃)). The product fractions were concentrated and lyophilized to afford N-[1-[1-[2-(benzyloxy)ethyl]-2-methyl-4-[2-(propan-2-yl)pyridin-3-yl]-1H-imidazol-5-yl]ethyl]-2-methylpropane-2-sulfinamide as a white solid (1.20 g, 58%). LCMS (ESI, m z) 483 [M+H]⁺.

### Step 5. 1-{1-[2-(benzyloxy)ethyl]-2-methyl-4-[2-(propan-2-yl)pyridin-3-yl]-1H-imidazol-5-yl}ethan-1-amine (HCl salt)

A mixture of N-[(1R)-1-[1-[2-(benzyloxy)ethyl]-2-methyl-4-[2-(propan-2-yl)pyridin-3-yl]-1H-imidazol-5-yl]ethyl]-2-methylpropane-2-sulfinamide (1.20 g, 2.49 mmol) and hydrochloric acid (5 mL, 4 M in 1,4-dioxane) in MeOH (10 mL) was stirred for 16 h at rt. The reaction mixture was concentrated under vacuum to afford 1-{1-[2-(benzyloxy)ethyl]-2-methyl-4-[2-(propan-2-yl)pyridin-3-yl]-1H-imidazol-5-yl}ethan-1-amine (HCl salt) as a white solid (900 mg, 96%). LCMS (ESI, m z) 379 [M+H]⁺.

### Step 6. 2-(5-(1-aminoethyl)-4-(2-isopropylpyridin-3-yl)-2-methyl-1H-imidazol-1-yl)ethan-1-ol

A mixture of 1-{1-[2-(benzyloxy)ethyl]-2-methyl-4-[2-(propan-2-yl)pyridin-3-yl]-1H-imidazol-5-yl}ethan-1-amine (HCl salt) (900 mg, 2.38 mmol), palladium on carbon (500 mg, 10 wt % palladium on active carbon) and concentrated hydrochloric acid (3 mL) in ethanol (15 mL) was stirred for 2 h at rt under hydrogen atmosphere (2-3 atm). The reaction mixture was filtered through a Celite pad and the filtrate was concentrated under vacuum and poured into water (10 mL). The resulting mixture was basified to pH 8 with saturated sodium bicarbonate and then extracted with ethyl acetate (2×20 mL). The combined organic layers were dried over anhydrous sodium sulfate, filtered, and concentrated under vacuum. The crude product was purified by C₁₈ reversed phase chromatography (eluting with 5% to 50% MeCN/water (0.05% NH₄HCO₃)). The product fractions were concentrated and lyophilized to afford 2-(5-(1-aminoethyl)-4-(2-isopropylpyridin-3-yl)-2-methyl-1H-imidazol-1-yl)ethan-1-ol as a white solid (500 mg, 73%). LCMS (ESI, m z) 289 [M+H]⁺.

### Step 7. tert-butyl (1-(1-(2-hydroxyethyl)-4-(2-isopropylpyridin-3-yl)-2-methyl-1H-imidazol-5-yl)ethyl)carbamate

A mixture of 2-(5-(1-aminoethyl)-4-(2-isopropylpyridin-3-yl)-2-methyl-1H-imidazol-1-yl)ethan-1-ol (500 mg, 1.73 mmol), TEA (0.72 mL, 5.18 mmol), di-tert-butyl dicarbonate (452 mg, 2.07 mmol) and DCM (20 mL) was stirred for 16 h at rt and then concentrated under vacuum. The resulting crude product was purified by silica gel chromatography (eluting with 0:100 to 10:90 MeOH/DCM) to afford tert-butyl (1-(1-(2-hydroxyethyl)-4-(2-isopropylpyridin-3-yl)-2-methyl-1H-imidazol-5-yl)ethyl)carbamate as a white solid (500 mg, 74%). LCMS (ESI, m z) 389 [M+H]⁺.

### Step 8. tert-butyl (1-(4-(2-isopropylpyridin-3-yl)-2-methyl-1-(2-oxoethyl)-1H-imidazol-5-yl)ethyl)carbamate

A solution of tert-butyl (1-(1-(2-hydroxyethyl)-4-(2-isopropylpyridin-3-yl)-2-methyl-1H-imidazol-5-yl)ethyl)carbamate (673 mg, 1.73 mmol) in DCM (20 mL) was treated with Dess-Martin periodinane (1.40 g, 3.30 mmol) and the resulting mixture was stirred for 2 days at rt. The reaction mixture was filtered and concentrated under vacuum. The resulting crude product was purified by silica gel chromatography (eluting with 0:100 to 10:90 MeOH/DCM) to afford tert-butyl (1-(4-(2-isopropylpyridin-3-yl)-2-methyl-1-(2-oxoethyl)-1H-imidazol-5-yl)ethyl)carbamate as yellow oil (500 mg, 75%). LCMS (ESI, m z) 387 [M+H]⁺.

### Step 9. 3-[3,8-dimethyl-5H,8H-imidazo[1,5-a]pyrazin-1-yl]-2-(propan-2-yl)pyridine (TFA salt)

A mixture of tert-butyl (1-(4-(2-isopropylpyridin-3-yl)-2-methyl-1-(2-oxoethyl)-1H-imidazol-5-yl)ethyl)carbamate (500 mg, 1.29 mmol), TFA (3 mL), and DCM (10 mL) was stirred for 16 h at rt and concentrated under vacuum to afford 3-[-3,8-dimethyl-5H,8H-imidazo[1,5-a]pyrazin-1-yl]-2-(propan-2-yl)pyridine (TFA salt) as yellow oil (300 mg, 65%). LCMS (ESI, m z) 269 [M+H]⁺.

### Step 10. 1-(2-isopropylpyridin-3-yl)-3,8-dimethyl-5,6,7,8-tetrahydroimidazo[1,5-a]pyrazine

A mixture of 3-[3,8-dimethyl-5H,8H-imidazo[1,5-a]pyrazin-1-yl]-2-(propan-2-yl)pyridine (TFA salt) (300 mg, 1.05 mmol), sodium cyanoborohydride (132 mg, 2.10 mmol) and MeOH (10 mL) was stirred for 1 h at rt. The reaction mixture was concentrated under vacuum. The resulting crude product was purified by Prep-TLC (eluting with 1:10 MeOH/DCM) to afford 1-(2-isopropylpyridin-3-yl)-3,8-dimethyl-5,6,7,8-tetrahydroimidazo[1,5-a]pyrazine as yellow oil (80 mg, 28%). LCMS (ESI, m z) 271 [M+H]⁺.

### Intermediate A-6. 1-(2-isopropylpyridin-3-yl)-3-(trifluoromethyl)-5,6,7,8-tetrahydroimidazo[1,5-a]pyrazine

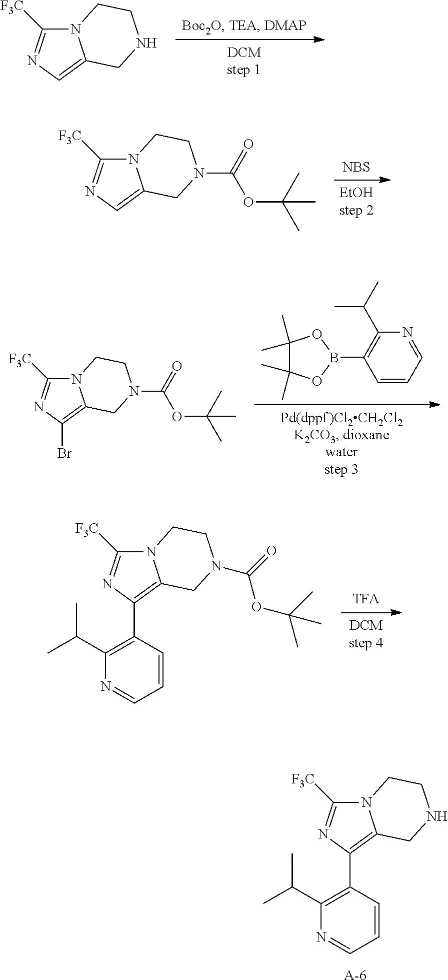

### Step 1. tert-butyl 3-(trifluoromethyl)-5,6-dihydroimidazo[1,5-a]pyrazine-7(8H)-carboxylate

A mixture of 3-(trifluoromethyl)-5,6,7,8-tetrahydroimidazo[1,5-a]pyrazine (1 g, 5.18 mmol, 1.00 equiv), DCM (10 mL), TEA (2.11 g, 20.90 mmol, 4.04 equiv), di-tert-butyl dicarbonate (1.71 g, 7.84 mmol, 1.51 equiv) and 4-dimethylaminopyridine (64 mg, 0.52 mmol, 0.10 equiv) was stirred for 18 h at 25° C. The reaction mixture was treated with water (20 mL) and then extracted with DCM (3×10 mL). The organic layers were combined, washed with saturated ammonium chloride solution (10 mL) and brine (10 mL), dried over anhydrous sodium sulfate, filtered, and concentrated under vacuum to afford 0.82 g (54%) of tert-butyl 3-(trifluoromethyl)-5,6-dihydroimidazo[1,5-a]pyrazine-7(8H)-carboxylate as a yellow oil. LCMS (ESI, m z) 292.3 [M+H]⁺.

### Step 2. tert-butyl 1-bromo-3-(trifluoromethyl)-5,6-dihydroimidazo[1,5-a]pyrazine-7(8H)-carboxylate

A mixture of tert-butyl 3-(trifluoromethyl)-5,6-dihydroimidazo[1,5-a]pyrazine-7(8H)-carboxylate (820 mg, 2.62 mmol, 1.00 equiv), ethanol (80 mL), and N-bromo-succinimide (1.00 g, 5.64 mmol, 2.15 equiv) was stirred for 1 h at 25° C. The reaction mixture was concentrated under vacuum and then dissolved with ethyl acetate (20 mL). The resulting solution was washed with sodium bicarbonate solution (10 mL) and brine (10 mL), dried over anhydrous sodium sulfate, filtered, and concentrated under vacuum. The residue was purified by silica gel chromatography (eluting with 0/100 to 30/70 ethyl acetate/petroleum ether) to afford 320 mg (33%) of tert-butyl 1-bromo-3-(trifluoromethyl)-5,6-dihydroimidazo[1,5-a]pyrazine-7(8H)-carboxylate as a yellow oil. LCMS (ESI, m z) 369.8, 371.8 [M+H]⁺.

### Step 3. tert-butyl 1-(2-isopropylpyridin-3-yl)-3-(trifluoromethyl)-5,6-dihydroimidazo[1,5-a]pyrazine-7(8H)-carboxylate

A mixture of tert-butyl 1-bromo-3-(trifluoromethyl)-5,6-dihydroimidazo[1,5-a]pyrazine-7(8H)-carboxylate (320 mg, 0.82 mmol, 1.00 equiv), 2-isopropyl-3-(4,4,5,5-tetramethyl-1,3,2-dioxaborolan-2-yl)pyridine (257 mg, 1.04 mmol, 1.27 equiv), Pd(dppf)Cl₂—CH₂Cl₂ (70.85 mg, 0.09 mmol, 0.11 equiv), potassium carbonate (359 mg, 2.60 mmol, 3.16 equiv), water (2 mL), and 1,4-dioxane (8 mL) was stirred for 18 h at 100° C. After cooling to rt, the reaction mixture was filtered and concentrated under vacuum. The residue was purified by silica gel chromatography (eluting with 2/1 ethyl acetate/petroleum ether) to afford 250 mg (74%) of tert-butyl 1-(2-isopropylpyridin-3-yl)-3-(trifluoromethyl)-5,6-dihydroimidazo[1,5-a]pyrazine-7(8H)-carboxylate as a yellow oil. LCMS (ESI, m z) 411.2 [M+H]⁺.

### Step 4. 1-(2-isopropylpyridin-3-yl)-3-(trifluoromethyl)-5,6,7,8-tetrahydroimidazo[1,5-a]pyrazine

A mixture of tert-butyl 1-(2-isopropylpyridin-3-yl)-3-(trifluoromethyl)-5,6-dihydroimidazo[1,5-a]pyrazine-7(8H)-carboxylate (250 mg, 0.58 mmol, 1.00 equiv), DCM (10 mL), and TFA (2 mL) was stirred for 1 h at 25° C. The reaction mixture was concentrated under vacuum, treated with MeOH (1 mL), and then basified with 7 M ammonia in MeOH to achieve pH 8. The resulting solution was concentrated under vacuum and the residue was purified by silica gel chromatography (eluting with 1/10 MeOH/DCM) to afford 180 mg (95%) of 1-(2-isopropylpyridin-3-yl)-3-(trifluoromethyl)-5,6,7,8-tetrahydroimidazo[1,5-a]pyrazine as a yellow oil. LCMS (ESI, m z) 311.2 [M+H]⁺.

### Intermediate A-7. 1-(2-isopropylpyridin-3-yl)-3,6-dimethyl-5,6,7,8-tetrahydroimidazo[1,5-a]pyrazine

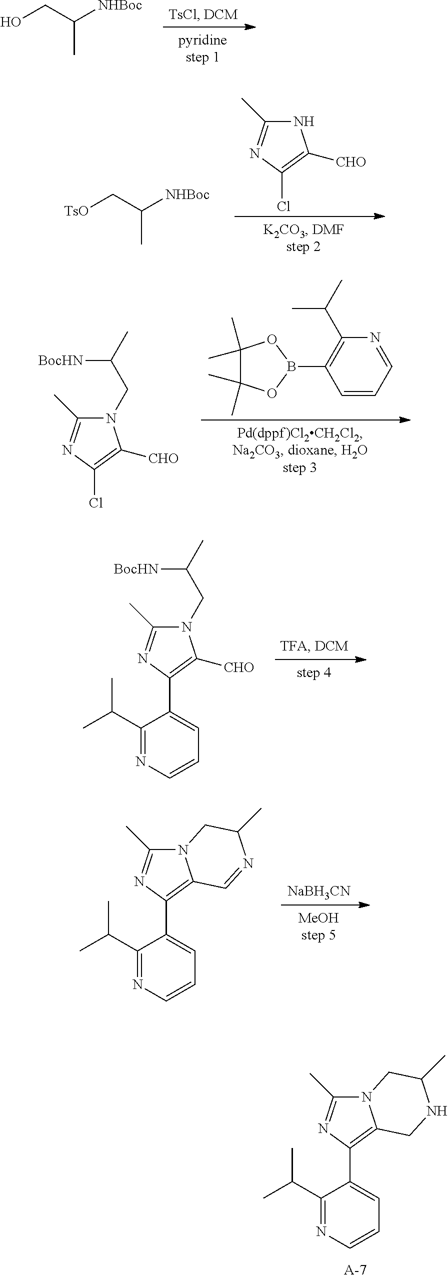

### Step 1. 2-((tert-butoxycarbonyl)amino)propyl 4-methylbenzenesulfonate

Into a 250 mL round-bottom flask was placed tert-butyl (1-hydroxypropan-2-yl)carbamate (10 g, 57.07 mmol, 1.00 equiv), DCM (100 mL) and pyridine (9 g, 113.78 mmol, 1.99 equiv). This was followed by the portion wise addition of 4-methylbenzene-1-sulfonyl chloride (12 g, 62.94 mmol, 1.10 equiv) with stirring at 0° C. The resulting solution was stirred overnight at rt. The reaction mixture was treated with water (200 mL) and then extracted with DCM (200 mL×3). The organic layers were combined, dried over anhydrous sodium sulfate, filtered and concentrated under vacuum. The residue was purified by silica gel chromatography (eluting with 0/100 to 30/70 ethyl acetate/petroleum ether) to afford 10 g (53%) of 2-((tert-butoxycarbonyl)amino)propyl 4-methylbenzenesulfonate as a white solid. LCMS (ESI, m z) 329.7 [M+H]⁺.

### Step 2. tert-butyl (1-(4-chloro-5-formyl-2-methyl-1H-imidazol-1-yl)propan-2-yl)carbamate

Into a 250 mL round-bottom flask was placed 4-chloro-2-methyl-1H-imidazole-5-carbaldehyde (2 g, 13.84 mmol, 1.00 equiv), 2-((tert-butoxycarbonyl)amino)propyl 4-methylbenzenesulfonate (10 g, 30.36 mmol, 2.19 equiv), potassium carbonate (3.8 g, 27.50 mmol, 1.99 equiv), and DMF (100 mL). The resulting mixture was stirred overnight at 50° C. After cooling to rt, the reaction mixture was treated with water (100 mL) and extracted with ethyl acetate (2×100 mL). The organic layers were combined, washed with brine (2×100 mL), dried over anhydrous sodium sulfate, filtered, and concentrated under vacuum. The residue was purified by silica gel chromatography (eluting with 0/100 to 70/30 ethyl acetate/petroleum ether) to afford 600 mg (14%) of tert-butyl (1-(4-chloro-5-formyl-2-methyl-1H-imidazol-1-yl)propan-2-yl)carbamate as a yellow solid. LCMS (ESI, m z) 301.7 [M+H]⁺.

### Step 3. tert-butyl (1-(5-formyl-4-(2-isopropylpyridin-3-yl)-2-methyl-1H-imidazol-1-yl)propan-2-yl)carbamate

Into a 100 mL round-bottom flask purged and maintained with an inert atmosphere of nitrogen was placed tert-butyl (1-(4-chloro-5-formyl-2-methyl-1H-imidazol-1-yl)propan-2-yl)carbamate (600 mg, 1.99 mmol, 1.00 equiv), 2-isopropyl-3-(4,4,5,5-tetramethyl-1,3,2-dioxaborolan-2-yl)pyridine (588 mg, 2.38 mmol, 1.20 equiv), Pd(dppf)Cl₂—CH₂Cl₂ (162 mg, 0.20 mmol, 0.10 equiv), sodium carbonate (421 mg, 3.97 mmol, 2.00 equiv), 1,4-dioxane (15 mL) and water (3 mL). The resulting mixture was stirred overnight at 100° C. After cooling to rt, the reaction mixture was filtered and concentrated under vacuum. The residue was purified by silica gel chromatography (eluting with 20/80 to 80/20 ethyl acetate/petroleum ether) to afford 550 mg (68%) of tert-butyl (1-(5-formyl-4-(2-isopropylpyridin-3-yl)-2-methyl-1H-imidazol-1-yl)propan-2-yl)carbamate as a yellow solid. LCMS (ESI, m z) 386.8 [M+H]⁺.

### Step 4. 1-(2-isopropylpyridin-3-yl)-3,6-dimethyl-5,6-dihydroimidazo[1,5-a]pyrazine

Into a 50 mL round-bottom flask was placed tert-butyl (1-(5-formyl-4-(2-isopropylpyridin-3-yl)-2-methyl-1H-imidazol-1-yl)propan-2-yl)carbamate (550 mg, 1.42 mmol, 1.00 equiv), DCM (6 mL), and TFA (1 mL). The resulting solution was stirred for 1 h at rt. The reaction mixture was concentrated under vacuum to afford 400 mg (crude) of 1-(2-isopropylpyridin-3-yl)-3,6-dimethyl-5,6-dihydroimidazo[1,5-a]pyrazine as a red oil. LCMS (ESI, m z) 268.6 [M+H]⁺.

### Step 5. 1-(2-isopropylpyridin-3-yl)-3,6-dimethyl-5,6,7,8-tetrahydroimidazo[1,5-a]pyrazine

Into a 50 mL round-bottom flask was placed 1-(2-isopropylpyridin-3-yl)-3,6-dimethyl-5,6-dihydroimidazo[1,5-a]pyrazine (400 mg, 1.40 mmol, 1.00 equiv) and MeOH (10 mL). This was followed by the portion wise addition of NaBH₃CN (176 mg, 2.80 mmol, 2.00 equiv) with stirring at 0° C. The resulting solution was stirred for 30 min at 0° C. The reaction mixture was treated with saturated NaHCO₃ solution (3 mL) and then extracted with ethyl acetate (3×10 mL). The organic layers were combined, dried over anhydrous sodium sulfate, filtered, and concentrated under vacuum. The residue was purified by silica gel chromatography (eluting with 0/100 to 10/90 MeOH/DCM) to afford 200 mg (53%) of 1-(2-isopropylpyridin-3-yl)-3,6-dimethyl-5,6,7,8-tetrahydroimidazo[1,5-a]pyrazine as a white solid. LCMS (ESI, m z) 270.7 [M+H]⁺.

The following intermediates were prepared using the procedure for Intermediate A-7 with the appropriate boronic ester or boronic acid used in step 3:

TABLE 3

| Intermediate | Name | Structure | LCMS (ESI, m/z) |
|---|---|---|---|
| A-8 | 1-(2-(difluoromethoxy)pyridin- 3-yl)-3,6-dimethyl-5,6,7,8- tetrahydroimidazo[1,5- a]pyrazine | 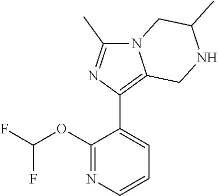 [SMILES: Cc1nc(-c2cccnc2OC(F)F)c2n1CC(C)NC2] | 295.1 [M + H]⁺ |
| A-9^{a} | 1-(2-isopropylpyridin-3-yl)-3,5- dimethyl-5,6,7,8- tetrahydroimidazo[1,5- a]pyrazine | 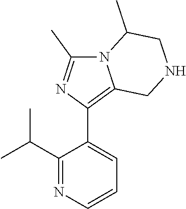 [SMILES: Cc1nc(-c2cccnc2C(C)C)c2n1C(C)CNC2] | 271.2 [M + H]⁺ |

^{a}Tert-butyl (2-hydroxypropyl)carbamate was substituted for (1-hydroxypropan-2-yl)carbamate in step 1.

### Intermediate A-10. 3-cyclopropyl-1-(2-isopropylpyridin-3-yl)-5,6,7,8-tetrahydroimidazo[1,5-a]pyrazine

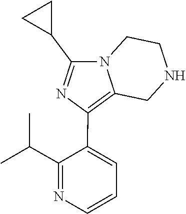 [SMILES: CC(C)c1ncccc1-c1nc(C2CC2)n2c1CNCC2]

Intermediate A-10 was prepared following the same procedure for Intermediate A-1, substituting ethyl cyclopropanecarbimidate for ethyl ethanecarboximidate hydrochloride in Step 1. LCMS (ESI, m z) 283.1 [M+H]⁺.

### Intermediate A-11. 1-(2-(difluoromethoxy)pyridin-3-yl)-3,8-dimethyl-5,6,7,8-tetrahydroimidazo[1,5-a]pyrazine

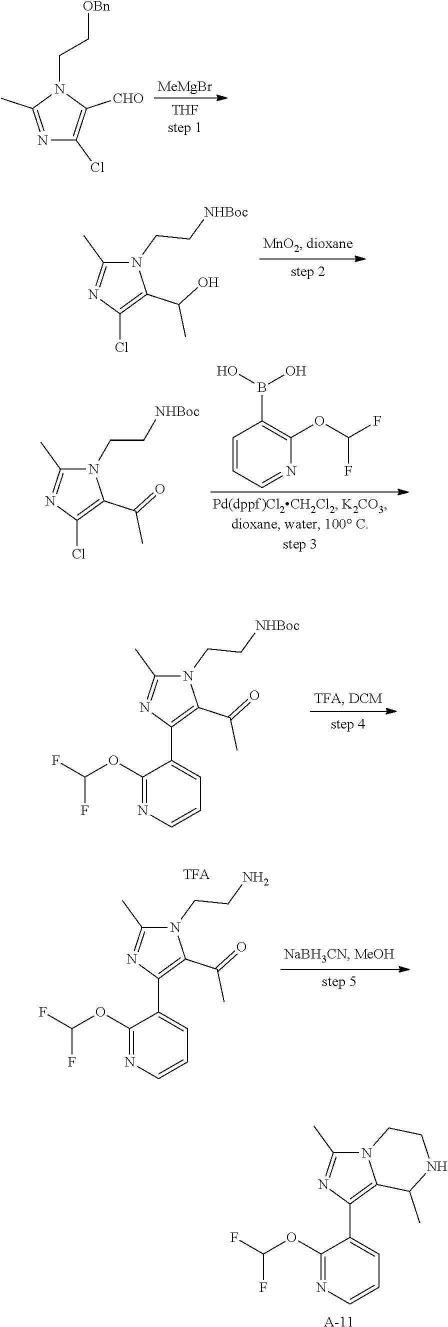

### Step 1. tert-butyl (2-(4-chloro-5-(1-hydroxyethyl)-2-methyl-1H-imidazol-1-yl)ethyl)carbamate

To a solution of 1-(2-(benzyloxy)ethyl)-4-chloro-2-methyl-1H-imidazole-5-carbaldehyde (700 mg, 2.43 mmol) in THE (10 mL) was added methylmagnesium bromide solution (2.43 mL, 3 M in THF) with stirring at 0° C. The resulting mixture was stirred for 3 h at rt. The reaction mixture was treated with water (10 mL) and then extracted with ethyl acetate (20 mL×2). The organic layers were combined, dried over anhydrous sodium sulfate, filtered, and concentrated under vacuum to afford tert-butyl (2-(4-chloro-5-(1-hydroxyethyl)-2-methyl-1H-imidazol-1-yl)ethyl)carbamate as a yellow solid (500 mg, crude). LCMS (ESI, m z) 304, 306 [M+H]⁺.

### Step 2. tert-butyl (2-(5-acetyl-4-chloro-2-methyl-1H-imidazol-1-yl)ethyl)carbamate

To a solution of tert-butyl (2-(4-chloro-5-(1-hydroxyethyl)-2-methyl-1H-imidazol-1-yl)ethyl)carbamate (500 mg, 1.65 mmol) in 1,4-dioxane (20 mL) was added manganese dioxide (2.86 g, 32.9 mmol). The resulting mixture was stirred for 3 days at 60° C. After cooling to rt, the reaction mixture was filtered and concentrated under vacuum. The resulting crude product was purified by silica gel chromatography (eluting with 0/100 to 70/30 ethyl acetate/petroleum ether) to afford tert-butyl (2-(5-acetyl-4-chloro-2-methyl-1H-imidazol-1-yl)ethyl)carbamate as an off-white solid (450 mg, 86%). LCMS (ESI, m z) 302, 304 [M+H]⁺.

### Step 3. tert-butyl (2-(5-acetyl-4-(2-(difluoromethoxy)pyridin-3-yl)-2-methyl-1H-imidazol-1-yl)ethyl)carbamate

To a solution of tert-butyl (2-(5-acetyl-4-chloro-2-methyl-1H-imidazol-1-yl)ethyl)carbamate (490 mg, 1.62 mmol) in 1,4-dioxane (8 mL) was added [2-(difluoromethoxy)pyridine-3-yl]boronic acid (460 mg, 2.43 mmol), Pd(dppf)Cl₂—CH₂Cl₂ (133 mg, 0.16 mmol), potassium carbonate (449 mg, 3.25 mmol), and water (1.6 mL). The resulting mixture was stirred for 16 h at 100° C. and then cooled to rt. The reaction mixture was poured into water (10 mL) and then extracted with ethyl acetate (3×20 mL). The combined organic layers were dried over anhydrous sodium sulfate, filtered, and concentrated under vacuum. The resulting crude product was purified by prep-TLC (eluting with 100:1 ethyl acetate/petroleum ether) to afford tert-butyl (2-(5-acetyl-4-(2-(difluoromethoxy)pyridin-3-yl)-2-methyl-1H-imidazol-1-yl)ethyl)carbamate as a brown solid (260 mg, 39%). LCMS (ESI, m z) 411 [M+H]⁺.

### Step 4. 1-(1-(2-aminoethyl)-4-(2-(difluoromethoxy)pyridin-3-yl)-2-methyl-1H-imidazol-5-yl)ethan-1-one (TFA salt)

To a solution of tert-butyl (2-(5-acetyl-4-(2-(difluoromethoxy)pyridin-3-yl)-2-methyl-1H-imidazol-1-yl)ethyl)carbamate (260 mg, 0.63 mmol) in DCM (5 mL) was added TFA (0.5 mL). The resulting mixture was stirred for 2 h at rt. The solution was concentrated under vacuum to afford 1-(1-(2-aminoethyl)-4-(2-(difluoromethoxy)pyridin-3-yl)-2-methyl-1H-imidazol-5-yl)ethan-1-one (TFA salt) as brown oil (240 mg, 89%). LCMS (ESI, m z) 311 [M+H]⁺.

### Step 5. 1-(2-(difluoromethoxy)pyridin-3-yl)-3,8-dimethyl-5,6,7,8-tetrahydroimidazo[1,5-a]pyrazine

To a solution of 1-(1-(2-aminoethyl)-4-(2-(difluoromethoxy)pyridin-3-yl)-2-methyl-1H-imidazol-5-yl)ethan-1-one (TFA salt) (240 mg, 0.77 mmol) in MeOH (5 mL) was added sodium cyanoborohydride (98 mg, 1.55 mmol) at 0° C. The resulting mixture was stirred for 0.5 h at rt and then concentrated under vacuum. The resulting crude product was purified by prep-TLC (eluting with 100:1 ethyl acetate/petroleum ether) to afford 1-(2-(difluoromethoxy)pyridin-3-yl)-3,8-dimethyl-5,6,7,8-tetrahydroimidazo[1,5-a]pyrazine as light yellow oil (130 mg, 57%). LCMS (ESI, m z) 295 [M+H]⁺.

### Intermediate A-12. 1-(2-isopropylpyridin-3-yl)-3-methoxy-5,6,7,8-tetrahydroimidazo[1,5-a]pyrazine

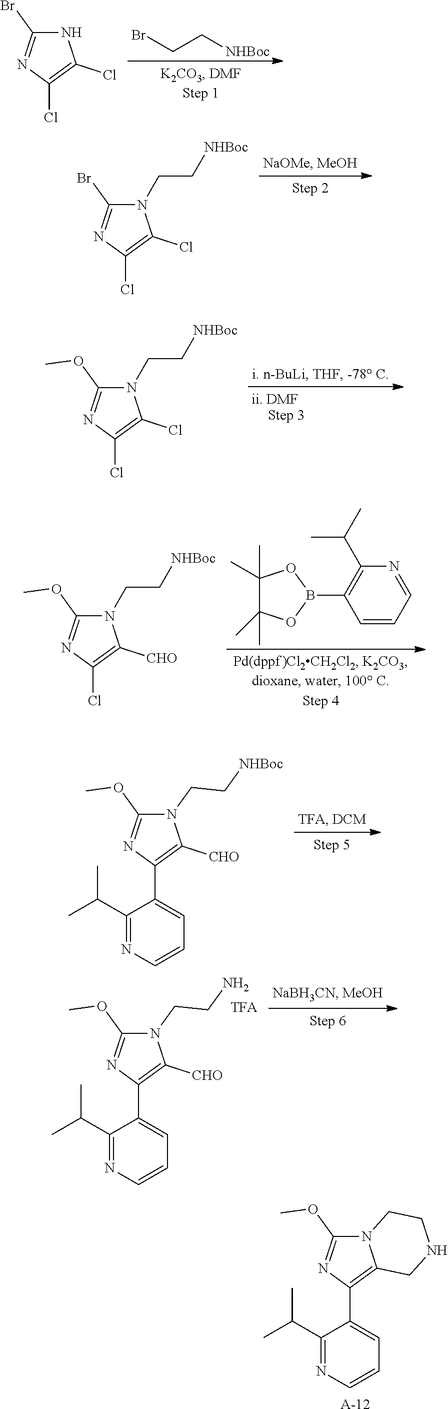

### Step 1. tert-butyl (2-(2-bromo-4,5-dichloro-1H-imidazol-1-yl)ethyl)carbamate

To a solution of 2-bromo-4,5-dichloro-1H-imidazole (5.00 g, 23.2 mmol) in DMF (70 mL) was added tert-butyl (2-bromoethyl)carbamate (5.70 g, 25.4 mmol), and potassium carbonate (9.60 g, 69.5 mmol). The resulting mixture was stirred for 16 h at 50° C. and cooled to rt. The reaction mixture was poured into water (80 mL) and then extracted with ethyl acetate (3×80 mL). The combined organic layers were dried over anhydrous sodium sulfate, filtered, and concentrated under vacuum. The resulting crude product was purified by silica gel chromatography (eluting with 0:100 to 50:50 ethyl acetate/petroleum ether) to afford tert-butyl (2-(2-bromo-4,5-dichloro-1H-imidazol-1-yl)ethyl)carbamate as a white solid (4.80 g, 58%). LCMS (ESI, m z) 358, 360, 362 [M+H]⁺.

### Step 2. tert-butyl (2-(4,5-dichloro-2-methoxy-1H-imidazol-1-yl)ethyl)carbamate

To a solution of tert-butyl (2-(2-bromo-4,5-dichloro-1H-imidazol-1-yl)ethyl)carbamate (1.50 g, 4.18 mmol) in MeOH (20 mL) was added sodium methoxide (2.26 g, 41.8 mmol). The resulting mixture was stirred for 3 days at 60° C. and cooled to rt. The reaction mixture was concentrated under vacuum and treated with water (15 mL). The resulting mixture was extracted with ethyl acetate (2×20 mL). The combined organic layers were washed with brine (10 mL), dried over anhydrous sodium sulfate, filtered, and concentrated under vacuum. The resulting crude product was purified by silica gel chromatography (eluting with 0:100 to 50:50 ethyl acetate/petroleum ether) to afford tert-butyl (2-(4,5-dichloro-2-methoxy-1H-imidazol-1-yl)ethyl)carbamate as a white solid (500 mg, 39%). LCMS (ESI, m z) 310, 312 [M+H]⁺.

### Step 3. tert-butyl (2-(4,5-dichloro-2-methoxy-1H-imidazol-1-yl)ethyl)carbamate

To a solution of tert-butyl (2-(4,5-dichloro-2-methoxy-1H-imidazol-1-yl)ethyl)carbamate (700 mg, 2.26 mmol) in THE (10 mL) was added n-BuLi (2.3 mL, 2.5 M in THF) dropwise with stirring at −78° C. The resulting mixture was stirred for 30 min at this temperature and was added DMF (0.70 mL, 9.03 mmol). The resulting mixture was stirred for another 1 h at −78° C. The reaction mixture was treated with saturated ammonium chloride solution (10 mL) and then extracted with ethyl acetate (3×10 mL). The combined organic layers were dried over anhydrous sodium sulfate, filtered, and concentrated under vacuum. The resulting crude product was purified by silica gel chromatography (eluting with 0:100 to 50:50 ethyl acetate/petroleum ether) to afford tert-butyl (2-(4,5-dichloro-2-methoxy-1H-imidazol-1-yl)ethyl)carbamate as a yellow solid (500 mg, 73%). LCMS (ESI, m z) 304, 306 [M+H]⁺.

### Step 4. tert-butyl (2-(5-formyl-4-(2-isopropylpyridin-3-yl)-2-methoxy-1H-imidazol-1-yl)ethyl)carbamate

To a solution of tert-butyl (2-(4,5-dichloro-2-methoxy-1H-imidazol-1-yl)ethyl)carbamate (500 mg, 1.65 mmol) in 1,4-dioxane (8 mL) and water (2 mL) was added 2-isopropyl-3-(4,4,5,5-tetramethyl-1,3,2-dioxaborolan-2-yl)pyridine (610 mg, 2.47 mmol), potassium carbonate (683 mg, 4.94 mmol), and Pd(dppf)Cl₂—CH₂Cl₂ (134 mg, 0.16 mmol). The resulting mixture was stirred overnight at 100° C. and cooled to rt. The reaction mixture was filtered and concentrated under vacuum. The resulting crude product was purified by prep-TLC (eluting with 100% EA) to afford tert-butyl (2-(5-formyl-4-(2-isopropylpyridin-3-yl)-2-methoxy-1H-imidazol-1-yl)ethyl)carbamate as a yellow solid (550 mg, 86%). LCMS (ESI, m z) 389 [M+H]⁺.

### Step 5. 1-(2-aminoethyl)-4-(2-isopropylpyridin-3-yl)-2-methoxy-1H-imidazole-5-carbaldehyde (TFA salt)

To a solution of tert-butyl (2-(5-formyl-4-(2-isopropylpyridin-3-yl)-2-methoxy-1H-imidazol-1-yl)ethyl)carbamate (550 mg, 1.42 mmol) in DCM (10 mL) was added TFA (1 mL). The resulting solution was stirred for 2 h at rt. The reaction mixture was concentrated under vacuum to afford 1-(2-aminoethyl)-4-(2-isopropylpyridin-3-yl)-2-methoxy-1H-imidazole-5-carbaldehyde (TFA salt) as brown oil (500 mg, crude). LCMS (ESI, m z) 289 [M+H]⁺

### Step 6. 1-(2-isopropylpyridin-3-yl)-3-methoxy-5,6,7,8-tetrahydroimidazo[1,5-a]pyrazine

To a solution of 1-(2-aminoethyl)-4-(2-isopropylpyridin-3-yl)-2-methoxy-1H-imidazole-5-carbaldehyde (TFA salt) (500 mg, 1.73 mmol) in MeOH (10 mL) was added sodium cyanoborohydride (218 mg, 3.47 mmol). The resulting mixture was stirred for 1.5 h at rt. The reaction mixture was poured into water (10 mL) and then extracted with ethyl acetate (3×10 mL). The combined organic layers were dried over anhydrous sodium sulfate, filtered, and concentrated under vacuum. The resulting crude product was purified by silica gel chromatography (eluting with 0:100 to 50:50 ethyl acetate/petroleum ether) to afford 1-(2-isopropylpyridin-3-yl)-3-methoxy-5,6,7,8-tetrahydroimidazo[1,5-a]pyrazine as a light yellow solid (400 mg, 85%). LCMS (ESI, m z) 273 [M+H]⁺.

### Intermediate A-13. 1-(2-isopropylpyridin-3-yl)-5,6,7,8-tetrahydroimidazo[1,5-a]pyrazine

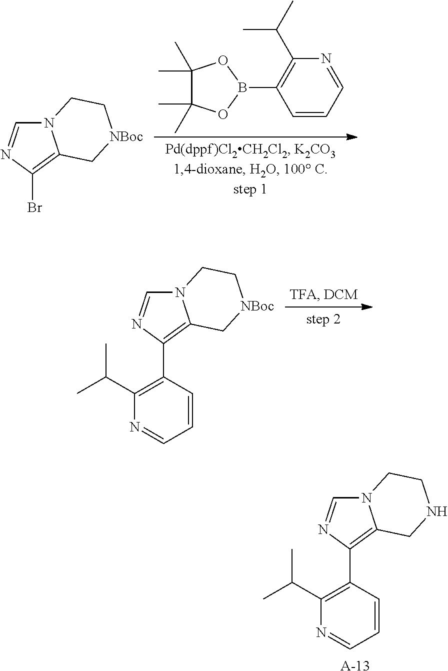

### Step 1. tert-butyl 1-(2-isopropylpyridin-3-yl)-5,6-dihydroimidazo[1,5-a]pyrazine-7(8H)-carboxylate

To a solution of tert-butyl 1-bromo-5,6-dihydroimidazo[1,5-a]pyrazine-7(8H)-carboxylate (500 mg, 1.65 mmol) in 1,4-dioxane (15 mL) was added potassium carbonate (451 mg, 3.26 mmol), 2-isopropyl-3-(4,4,5,5-tetramethyl-1,3,2-dioxaborolan-2-yl)pyridine (615 mg, 2.49 mmol), water (4 mL), and Pd(dppf)Cl₂—CH₂Cl₂ (136 mg, 0.17 mmol). The resulting mixture was stirred for 16 h at 100° C. After cooling to rt, the reaction mixture was poured into water (15 mL) and then extracted with ethyl acetate (3×25 mL). The combined organic layers were dried over anhydrous sodium sulfate, filtered, and concentrated under vacuum. The resulting crude product was purified by silica gel chromatography (eluting with 0:100 to 100:0 ethyl acetate/petroleum ether) to afford tert-butyl 1-(2-isopropylpyridin-3-yl)-5,6-dihydroimidazo[1,5-a]pyrazine-7(8H)-carboxylate as a yellow solid (400 mg, 71%). LCMS (ESI, m z) 343 [M+H]⁺.

### Step 2. 1-(2-isopropylpyridin-3-yl)-5,6,7,8-tetrahydroimidazo[1,5-a]pyrazine

To a solution of tert-butyl 1-(2-isopropylpyridin-3-yl)-5,6-dihydroimidazo[1,5-a]pyrazine-7(8H)-carboxylate (400 mg, 1.17 mmol) in DCM (15 mL), was added TFA (0.26 mL, 3.51 mmol). The resulting solution was stirred for 2 h at rt and concentrated under vacuum. The resulting mixture was basified to pH 8 with saturated potassium carbonate solution and then extracted with DCM (2×30 mL). The combined organic layers were dried over anhydrous sodium sulfate, filtered, and concentrated under vacuum to afford 1-(2-isopropylpyridin-3-yl)-5,6,7,8-tetrahydroimidazo[1,5-a]pyrazine as a yellow solid (250 mg, 88%). LCMS (ESI, m z) 243 [M+H]⁺.

### Intermediate B-1. 2-(4-bromophenyl)-1-methyl-4-(trifluoromethyl)-1H-imidazole

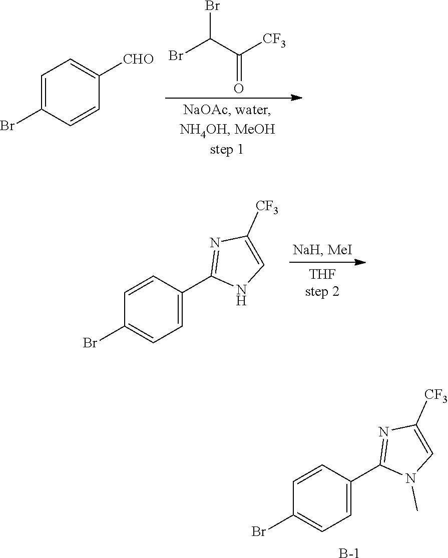

### Step 1. 2-(4-bromophenyl)-4-(trifluoromethyl)-1H-imidazole

A mixture of 3,3-dibromo-1,1,1-trifluoropropan-2-one (36.4 g, 133 mmol) and sodium acetate (22.2 g, 270 mmol) in water (58 mL) was stirred for 1 h at 100° C. then cooled to rt. A mixture of 4-bromobenzaldehyde (20.0 g, 107 mmol), MeOH (231 mL), and concentrated ammonium hydroxide (69 mL) was added, and the resulting mixture was stirred for 18 h at 25° C. The reaction mixture was concentrated and the precipitate was collected by filtration and washed with ethyl acetate/petroleum ether (1/5). The solids were collected and dried under vacuum to afford 2-(4-bromophenyl)-4-(trifluoromethyl)-1H-imidazole as a white solid (16 g, 51%). LCMS (ESI, m z) 291, 293 [M+H]⁺.

### Step 2. 2-(4-bromophenyl)-1-methyl-4-(trifluoromethyl)-1H-imidazole

A mixture of 2-(4-bromophenyl)-4-(trifluoromethyl)-1H-imidazole (16.0 g, 53.0 mmol) in DMF (150 mL) at 0° C. was treated portionwise with sodium hydride (4.40 g, 110 mmol, 60% dispersion in mineral oil). After 0.5 h at 0° C., iodomethane (3.76 mL, 60.5 mmol) was added and the resulting mixture was allowed to warm to 25° C. gradually and then stirred for 18 h. The reaction mixture was treated with water (300 mL) and then extracted with ethyl acetate (5×100 mL). The organic layers were combined, dried over anhydrous sodium sulfate, filtered and concentrated under vacuum. The resulting crude product was purified by silica gel chromatography (eluting with 0/100 to 10/90 ethyl acetate/petroleum ether) to afford 2-(4-bromophenyl)-1-methyl-4-(trifluoromethyl)-1H-imidazole as a colorless oil (10 g, 61%). LCMS (ESI, m z) 305, 307 [M+H]⁺.

### Intermediate B-2. 2-(4-bromophenyl)-1-(oxetan-3-yl)-4-(trifluoromethyl)-1H-imidazole

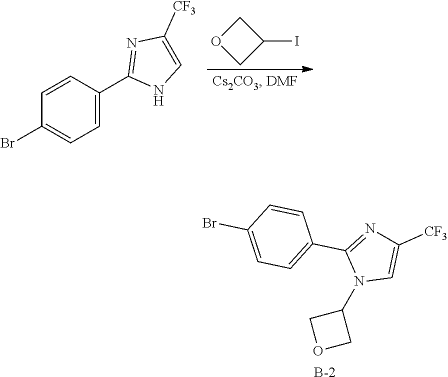

A mixture of 2-(4-bromophenyl)-4-(trifluoromethyl)-1H-imidazole (1.00 g, 3.44 mmol) and cesium carbonate (2.79 g, 8.56 mmol) in DMF (10 mL) was treated with 3-iodooxetane (1.26 g, 6.85 mmol), and the mixture was stirred for 72 h at 110° C. and cooled to rt. The reaction mixture was poured into water (20 mL) and then extracted with ethyl acetate (2×8 mL). The combined organic layers were dried over anhydrous sodium sulfate, filtered, and concentrated under vacuum. The resulting crude product was purified by silica gel chromatography (eluting with 0:100 to 30:70 ethyl acetate/petroleum ether) to afford 2-(4-bromophenyl)-1-(oxetan-3-yl)-4-(trifluoromethyl)-1H-imidazole as a yellow oil (200 mg, 17%). LCMS (ESI, m z) 347, 349 [M+H]⁺.

### Intermediate B-3. 2-chloro-5-(1-methyl-4-(trifluoromethyl)-H-imidazol-2-yl)pyridine

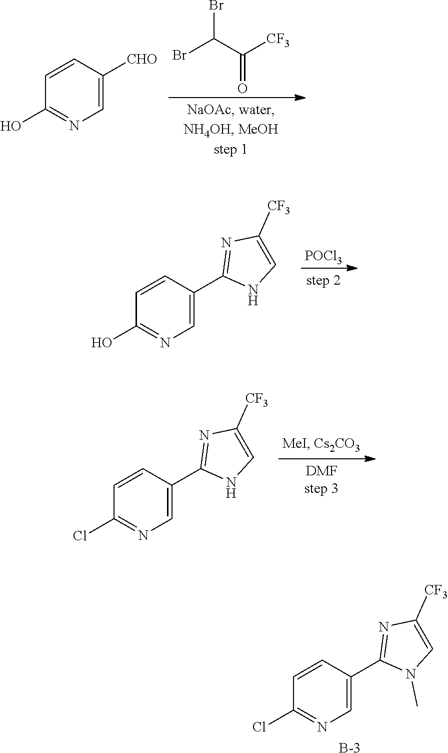

### Step 1. 5-(4-(trifluoromethyl)-1H-imidazol-2-yl)pyridin-2-ol

A mixture of 3,3-dibromo-1,1,1-trifluoropropan-2-one (13.6 g, 49.9 mmol) and sodium acetate (8.3 g, 101 mmol) in water (22 mL) was stirred for 1 h at 100° C. After cooling to rt, sequentially was added 6-hydroxynicotinaldehyde (5.00 g, 40.2 mmol), MeOH (86 mL), and concentrated ammonium hydroxide (26 mL). The resulting mixture was stirred for 18 h at rt then was concentrated under vacuum. The precipitate was collected by filtration and slurried in ethyl acetate/petroleum ether (1/3). The solids were collected by filtration and dried under vacuum to afford 5-(4-(trifluoromethyl)-1H-imidazol-2-yl)pyridin-2-ol as a yellow solid (4.2 g, 46%). LCMS (ESI, m z) 230 [M+H]⁺.

### Step 2. 2-chloro-5-(4-(trifluoromethyl)-1H-imidazol-2-yl)pyridine

A mixture of 5-(4-(trifluoromethyl)-1H-imidazol-2-yl)pyridin-2-ol (800 mg, 3.32 mmol) and phosphoroyl trichloride (8 mL) was stirred for 2 h at 90° C. then cooled to rt and concentrated under vacuum. The residue was treated with saturated sodium bicarbonate solution (10 mL) and was extracted with ethyl acetate (20 mL×3). The organic layers were combined, dried over anhydrous sodium sulfate, filtered, and concentrated under vacuum to afford 2-chloro-5-(4-(trifluoromethyl)-1H-imidazol-2-yl)pyridine as a yellow solid (860 mg, crude). LCMS (ESI, m z) 248, 250 [M+H]⁺.

### Step 3. 2-chloro-5-(1-methyl-4-(trifluoromethyl)-1H-imidazol-2-yl)pyridine

A solution of 2-chloro-5-(4-(trifluoromethyl)-1H-imidazol-2-yl)pyridine (500 mg, 2.02 mmol) in DMF (10 mL) was treated with cesium carbonate (1.30 g, 3.99 mmol) and cooled to 0° C. Iodomethane (0.13 mL, 2.01 mmol) was added dropwise with stirring at 0° C., and the resulting mixture was stirred for 1 h at rt. The reaction mixture was treated with water (20 mL) and then extracted with ethyl acetate (3×20 mL). The organic layers were combined, dried over anhydrous sodium sulfate, filtered, and concentrated under vacuum. The resulting crude product was purified by silica gel chromatography (eluting with 1/3 ethyl acetate/petroleum ether) to afford 2-chloro-5-(1-methyl-4-(trifluoromethyl)-1H-imidazol-2-yl)pyridine as a white solid (320 mg, 57%). LCMS (ESI, m z) 262, 264 [M+H]⁺.

### Intermediate B-4. 2-(4-bromophenyl)-1-cyclopropyl-4-(trifluoromethyl)-1H-imidazole

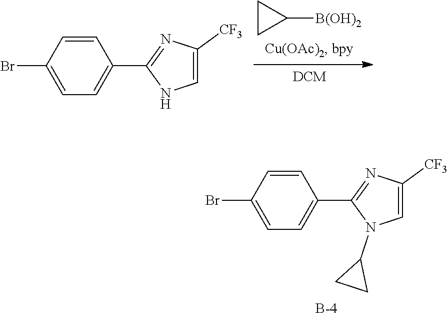

A mixture of 2-(4-bromophenyl)-4-(trifluoromethyl)-1H-imidazole (300 mg, 1.03 mmol), copper (II) acetate (565 mg, 3.11 mmol), 2,2′-bipyridine (2 mL) and cyclopropylboronic acid (270 mg, 3.11 mmol) in DCM (10 mL) was stirred for 3 days at rt. The reaction mixture was poured into water (15 mL) and then extracted with ethyl acetate (3×20 mL). The combined organic layers were dried over anhydrous sodium sulfate, filtered, and concentrated under vacuum. The resulting crude product was purified by silica gel chromatography (eluting with 0:100 to 20:80 ethyl acetate/petroleum ether) to afford 2-(4-bromophenyl)-1-cyclopropyl-4-(trifluoromethyl)-1H-imidazole as brown oil (276 mg, 81%). LCMS (ESI, m z) 331, 333 [M+H]⁺.

### Intermediate B-5. 2-(4-bromophenyl)-1-(1-methylazetidin-3-yl)-4-(trifluoromethyl)-1H-imidazole

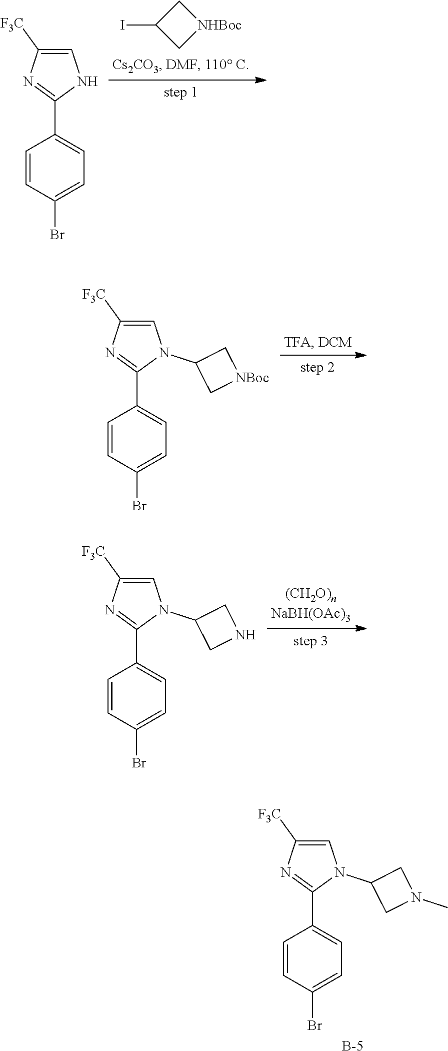

### Step 1. tert-butyl 3-(2-(4-bromophenyl)-4-(trifluoromethyl)-1H-imidazol-1-yl)azetidine-1-carboxylate

A mixture of 2-(4-bromophenyl)-4-(trifluoromethyl)-1H-imidazole (1.00 g, 3.44 mmol), tert-butyl 3-iodoazetidine-1-carboxylate (2.92 g, 10.3 mmol), and cesium carbonate (3.36 g, 10.3 mmol) in DMF (20 mL) was stirred for 18 h at 110° C. and then cooled to rt. The mixture was poured into water (20 mL) and then extracted with ethyl acetate (3×30 mL). The combined organic layers were dried over anhydrous sodium sulfate, filtered, and concentrated under vacuum. The resulting crude product was purified by silica gel chromatography (eluting with 0:100 to 60:40 ethyl acetate/petroleum ether) to afford tert-butyl 3-(2-(4-bromophenyl)-4-(trifluoromethyl)-1H-imidazol-1-yl)azetidine-1-carboxylate as orange oil (900 mg, 59%). LCMS (ESI, m z) 446, 448 [M+H]⁺.

### Step 2. 1-(azetidin-3-yl)-2-(4-bromophenyl)-4-(trifluoromethyl)-1H-imidazole

To a solution of tert-butyl 3-(2-(4-bromophenyl)-4-(trifluoromethyl)-1H-imidazol-1-yl)azetidine-1-carboxylate (900 mg, 2.02 mmol) in DCM (10 mL) was added TFA (2 mL). The resulting solution was stirred for 3 h at rt and concentrated under vacuum. The resulting mixture was basified to pH 8 with ammonia (7 M in MeOH) and then concentrated under vacuum to afford 1-(azetidin-3-yl)-2-(4-bromophenyl)-4-(trifluoromethyl)-1H-imidazole as brown oil (670 mg, crude). LCMS (ESI, m z) 346, 348 [M+H]⁺.

### Step 3. 2-(4-bromophenyl)-1-(1-methylazetidin-3-yl)-4-(trifluoromethyl)-1H-imidazole

To a solution of 1-(azetidin-3-yl)-2-(4-bromophenyl)-4-(trifluoromethyl)-1H-imidazole (700 mg, 2.02 mmol) in DCM (10 mL) was added paraformaldehyde (128 mg, 1.42 mmol). The resulting solution was stirred for 30 min and then sodium triacetoxyborohydride (1.28 g, 6.07 mmol) was added. The resulting mixture was stirred for 18 h at rt. The reaction mixture was poured into water (10 mL) and then extracted with ethyl acetate (3×10 mL). The combined organic layers were dried over anhydrous sodium sulfate, filtered, and concentrated under vacuum. The resulting crude product was purified by silica gel chromatography (eluting with 0:100 to 80:20 ethyl acetate/petroleum ether) to afford 2-(4-bromophenyl)-1-(1-methylazetidin-3-yl)-4-(trifluoromethyl)-1H-imidazole as a light yellow solid (120 mg, 16%). LCMS (ESI, m z) 360, 362 [M+H]⁺.

### Intermediate B-6. 2-(4-bromophenyl)-1-(2-methoxyethyl)-4-(trifluoromethyl)-1H-imidazole

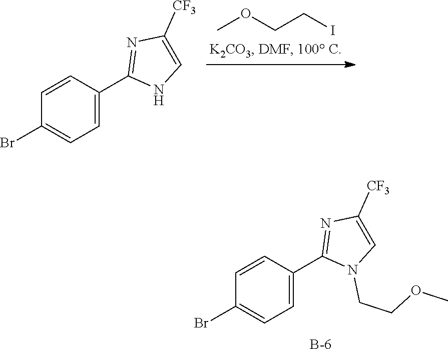

A mixture of 2-(4-bromophenyl)-4-(trifluoromethyl)-1H-imidazole (500 mg, 1.72 mmol), 1-iodo-2-methoxyethane (479 mg, 2.58 mmol), and potassium carbonate (475 mg, 3.44 mmol) in DMF (8 mL) was stirred for 16 h at 100° C. and cooled to rt. The reaction mixture was poured into water (10 mL) and then extracted with ethyl acetate (3×10 mL). The combined organic layers were dried over anhydrous sodium sulfate, filtered, and concentrated under vacuum. The resulting crude product was purified by silica gel chromatography (eluting with 0:100 to 40:60 ethyl acetate/petroleum ether) to afford 2-(4-bromophenyl)-1-(2-methoxyethyl)-4-(trifluoromethyl)-1H-imidazole as orange oil (500 mg, 83%). LCMS (ESI, m z) 349, 351 [M+H]⁺.

### Intermediate B-7. 4-(1,4-dimethyl-1H-imidazol-2-yl)phenyl trifluoromethanesulfonate

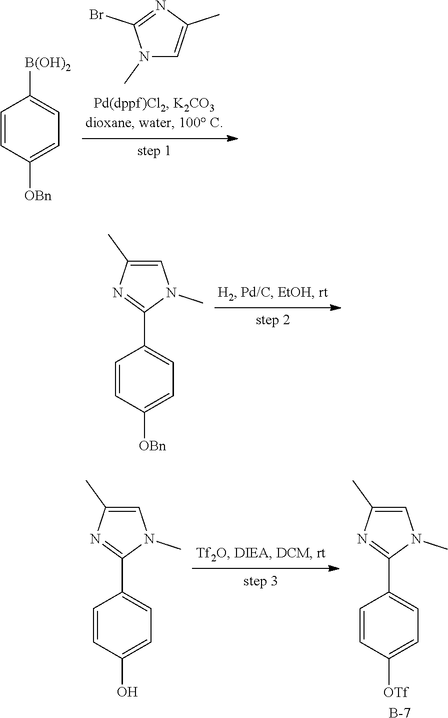

### Step 1. 2-(4-(benzyloxy)phenyl)-1,4-dimethyl-1H-imidazole

To a solution of (4-(benzyloxy)phenyl)boronic acid (1.30 g, 5.70 mmol) in 1,4-dioxane (20 mL) was added 2-bromo-1,4-dimethyl-1H-imidazole (1.00 g, 5.71 mmol), Pd(dppf)Cl₂ (417 mg, 0.58 mmol), potassium carbonate (1.60 g, 11.6 mmol), and water (6 mL). The resulting mixture was stirred for 18 h at 100° C. and then cooled to rt. The reaction mixture was poured into water (20 mL) and then extracted with ethyl acetate (3×30 mL). The combined organic layers were dried over anhydrous sodium sulfate, filtered, and concentrated under vacuum. The resulting crude product was purified by silica gel chromatography (eluting with 0:100 to 40:60 ethyl acetate/petroleum ether) to afford 2-(4-(benzyloxy)phenyl)-1,4-dimethyl-1H-imidazole as a black solid (1.00 g, 63%). LCMS (ESI, m z) 279 [M+H]⁺.

### Step 2. 4-(1,4-dimethyl-1H-imidazol-2-yl)phenol

To a solution of 2-(4-(benzyloxy)phenyl)-1,4-dimethyl-1H-imidazole (500 mg, 1.80 mmol) in ethanol (20 mL) was added palladium carbon (200 mg, 10 wt % palladium on active carbon). The resulting mixture was stirred for 3 h at rt under hydrogen atmosphere (2-3 atm). The reaction mixture was filtered and concentrated under vacuum. The resulting crude product was purified by prep-TLC (eluting with 2:1 ethyl acetate/petroleum ether) to afford 4-(1,4-dimethyl-1H-imidazol-2-yl)phenol as a yellow solid (300 mg, 88%). LCMS (ESI, m z) 189 [M+H]⁺.

### Step 3. 4-(1,4-dimethyl-1H-imidazol-2-yl)phenyl trifluoromethanesulfonate

To a solution of 4-(1,4-dimethyl-1H-imidazol-2-yl)phenol (300 mg, 1.59 mmol) in DCM (10 mL) was added DIEA (0.80 mL, 4.78 mmol) and trifluoromethanesulfonic anhydride (0.80 mL, 4.75 mmol) at 0° C. The resulting mixture was stirred for 16 h at rt. The reaction mixture was poured into water (10 mL) and then extracted with ethyl acetate (3×10 mL). The combined organic layers were dried over anhydrous sodium sulfate, filtered, and concentrated under vacuum. The resulting crude product was purified by prep-TLC (eluting with 1:1 ethyl acetate/petroleum ether) to afford 4-(1,4-dimethyl-1H-imidazol-2-yl)phenyl trifluoromethanesulfonate as yellow oil (270 mg, 53%). LCMS (ESI, m z) 321 [M+H]⁺.

### Intermediate B-8. 2-(4-bromophenyl)-4-chloro-1-methyl-1H-imidazole

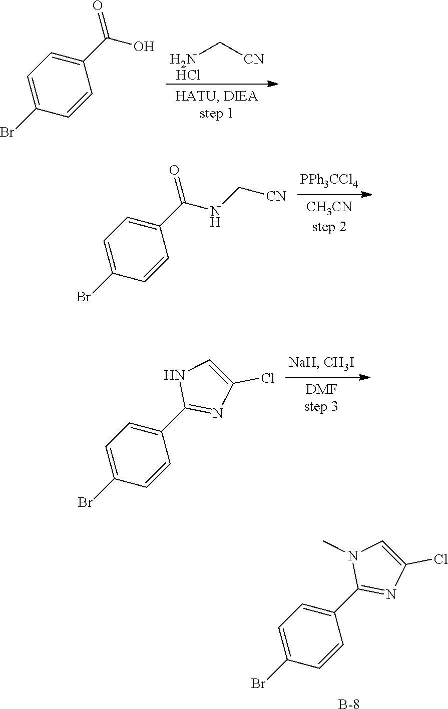

### Step 1. 4-bromo-N-(cyanomethyl)benzamide

To a solution of 4-bromobenzoic acid (3.00 g, 14.8 mmol) in DMF (50 mL) was added 2-aminoacetonitrile hydrochloride (2.10 g, 22.7 mmol), HATU (8.55 g, 22.3 mmol), and DIEA (4.94 mL, 29.9 mmol). The resulting mixture was stirred for 18 h at rt. The reaction mixture was poured into water (60 mL) and then extracted with ethyl acetate (3×100 mL). The combined organic layers were dried over anhydrous sodium sulfate, filtered, and concentrated under vacuum. The resulting crude product was purified by silica gel chromatography (eluting with 0:100 to 50:50 ethyl acetate/petroleum ether) to afford 4-bromo-N-(cyanomethyl)benzamide as a yellow solid (1.00 g, 28%). LCMS (ESI, m z) 239, 241 [M+H]⁺.

### Step 2. 2-(4-bromophenyl)-4-chloro-1H-imidazole

To a solution of 4-bromo-N-(cyanomethyl)benzamide (1.00 g, 3.89 mmol) in acetonitrile (30 mL) was added carbon tetrachloride (1.01 mL, 10.5 mmol) and triphenylphosphine (2.74 g, 10.4 mmol). The resulting mixture was stirred for 18 h at 50° C., then cooled to rt and concentrated under vacuum. The resulting mixture was poured into saturated sodium bicarbonate solution (30 mL) and then extracted with ethyl acetate (3×50 mL). The combined organic layers were dried over anhydrous sodium sulfate, filtered, and concentrated under vacuum. The resulting crude product was purified by silica gel chromatography (eluting with 0:100 to 50:50 ethyl acetate/petroleum ether) to afford 2-(4-bromophenyl)-4-chloro-1H-imidazole as a yellow solid (400 mg, 40%). LCMS (ESI, m z) 257, 259, 261 [M+H]⁺.

### Step 3. 2-(4-bromophenyl)-4-chloro-1-methyl-1H-imidazole

To a solution of 2-(4-bromophenyl)-4-chloro-1H-imidazole (400 mg, 1.44 mmol) in DMF (10 mL) was added sodium hydride (94 mg, 2.35 mmol, 60% dispersion in mineral oil) at 0° C. The resulting mixture was stirred for 30 min at 0° C. and then iodomethane (0.15 mL, 2.33 mmol) was added. The resulting mixture was stirred for 18 h at rt. The reaction mixture was poured into water (10 mL) and then extracted with ethyl acetate (3×20 mL). The combined organic layers were dried over anhydrous sodium sulfate, filtered, and concentrated under vacuum. The resulting crude product was purified by silica gel chromatography (eluting with 0:100 to 50:50 ethyl acetate/petroleum ether) to afford 2-(4-bromophenyl)-4-chloro-1-methyl-1H-imidazole as yellow oil (260 mg, 66%). LCMS (ESI, m z) 271, 273, 275 [M+H]⁺.

### Intermediate B-9. 4-(4-cyclopropyl-1-methyl-1H-imidazol-2-yl)phenyl trifluoromethanesulfonate

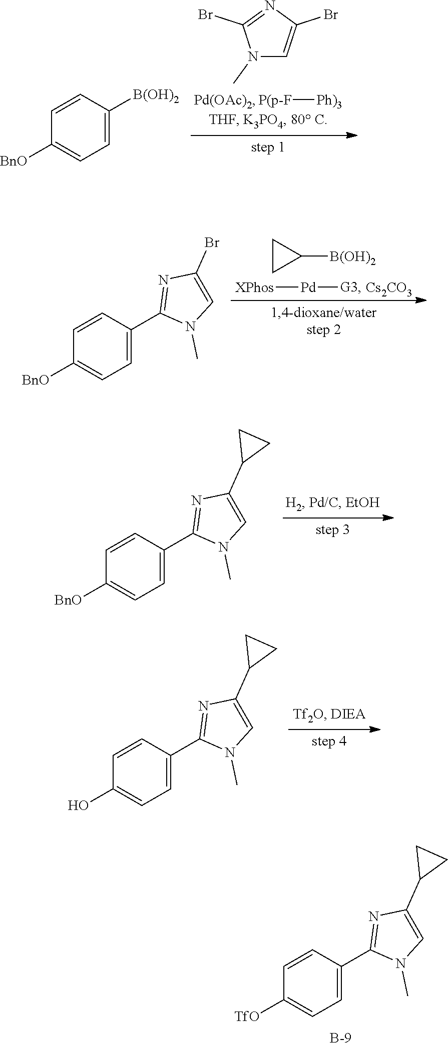

### Step 1. 2-(4-(benzyloxy)phenyl)-4-bromo-1-methyl-1H-imidazole

To a solution of (4-(benzyloxy)phenyl)boronic acid (4.90 g, 21.5 mmol) in THF (50 mL) was added 2,4-dibromo-1-methyl-1H-imidazole (4.65 g, 19.4 mmol), tris(4-fluorophenyl)phosphane (617 mg, 1.95 mmol), palladium(II) acetate (219 mg, 0.98 mmol) and potassium phosphate (8.28 g, 39.0 mmol). The resulting mixture was stirred for 12 h at 80° C. and cooled to rt. The reaction mixture was filtered and concentrated under vacuum. The resulting crude product was purified by silica gel chromatography (eluting with 0:100 to 50:50 ethyl acetate/petroleum ether) to afford 2-(4-(benzyloxy)phenyl)-4-bromo-1-methyl-1H-imidazole as light yellow solid (6.20 g, 84%). LCMS (ESI, m z) 343, 345 [M+H]⁺.

### Step 2. 2-(4-(benzyloxy)phenyl)-4-cyclopropyl-1-methyl-1H-imidazole

To a solution of 2-(4-(benzyloxy)phenyl)-4-bromo-1-methyl-1H-imidazole (1.00 g, 2.91 mmol) in 1,4-dioxane (5 mL) and water (1 mL) in a microwave tube was added cyclopropylboronic acid (377 mg, 4.39 mmol), XPhos-Pd-G3 (272 mg, 0.32 mmol), and cesium carbonate (1.90 g, 5.83 mmol). The resulting mixture was irradiated with microwave for 3 h at 110° C. and cooled to rt. The reaction mixture was poured into water (5 mL) and then extracted with ethyl acetate (3×10 mL). The combined organic layers were dried over anhydrous sodium sulfate, filtered, and concentrated under vacuum. The resulting crude product was purified by prep-TLC (eluting with 1:1 ethyl acetate/petroleum ether) to afford 2-(4-(benzyloxy)phenyl)-4-cyclopropyl-1-methyl-1H-imidazole as yellow oil (140 mg, 16%). LCMS (ESI, m z) 305 [M+H]⁺.

### Step 3. 4-(4-cyclopropyl-1-methyl-1H-imidazol-2-yl)phenol

To a solution of 2-(4-(benzyloxy)phenyl)-4-cyclopropyl-1-methyl-1H-imidazole (160 mg, 0.53 mmol) in ethanol (5 mL) was added palladium carbon (64 mg, 10 wt % palladium on active carbon). The resulting mixture was stirred for 3 h at rt under hydrogen atmosphere (2-3 atm). The reaction mixture was filtered and concentrated under vacuum. The resulting crude product was purified by prep-TLC (eluting with 99:1 ethyl acetate/petroleum ether) to afford 4-(4-cyclopropyl-1-methyl-1H-imidazol-2-yl)phenol as a black solid (80 mg, 71%). LCMS (ESI, m z) 215 [M+H]⁺.

### Step 4. 4-(4-cyclopropyl-1-methyl-1H-imidazol-2-yl)phenyl trifluoromethanesulfonate

To a solution of 4-(4-cyclopropyl-1-methyl-1H-imidazol-2-yl)phenol (100 mg, 0.47 mmol) in DCM (10 mL) was added DIEA (0.24 mL, 1.40 mmol) and (trifluoromethane)sulfonyl trifluoromethanesulfonate (0.08 mL, 0.47 mmol). The resulting solution was stirred for 2 h at rt and poured into water (3 mL). The resulting mixture was extracted with ethyl acetate (3×10 mL). The combined organic layers were dried over anhydrous sodium sulfate, filtered, and concentrated under vacuum. The resulting crude product was purified by prep-TLC (eluting with 1:10 ethyl acetate/petroleum ether) to afford 4-(4-cyclopropyl-1-methyl-1H-imidazol-2-yl)phenyl trifluoromethanesulfonate as yellow oil (70 mg, 43%). LCMS (ESI, m z) 347 [M+H]⁺.

### Intermediate B-10. 3-chloro-1-(4-iodophenyl)-5-methyl-1H-pyrazole and 5-chloro-1-(4-iodophenyl)-3-methyl-1H-pyrazole

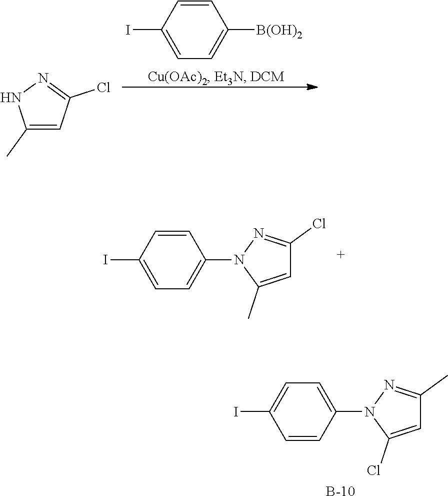

To a solution of 3-chloro-5-methyl-1H-pyrazole (500 mg, 4.29 mmol) in DCM (10 mL) was added (4-iodophenyl)boronic acid (2.14 g, 8.64 mmol), TEA (1.20 mL, 8.61 mmol), and copper(II) acetate (585 mg, 3.22 mmol). The resulting mixture was stirred for 3 h at rt under air atmosphere. The reaction mixture was treated with brine (15 mL) and then extracted with ethyl acetate (3×10 mL). The combined organic layers were dried over anhydrous sodium sulfate, filtered, and concentrated under vacuum. The resulting crude product was purified by prep-TLC (eluting with 2:1 ethyl acetate/petroleum ether) to afford the mixture of 3-chloro-1-(4-iodophenyl)-5-methyl-1H-pyrazole and 5-chloro-1-(4-iodophenyl)-3-methyl-1H-pyrazole as colorless oil (600 mg, 44%, ˜4:3 ratio based on LCMS). LCMS (ESI, m z) 319, 321 [M+H]⁺.

### Synthesis of Disclosed Compounds

### Compound 1. 1-(2-isopropylpyridin-3-yl)-3-methyl-7-(4-(1-methyl-4-(trifluoromethyl)-1H-imidazol-2-yl)phenyl)-5,6,7,8-tetrahydroimidazo[1,5-a]pyrazine

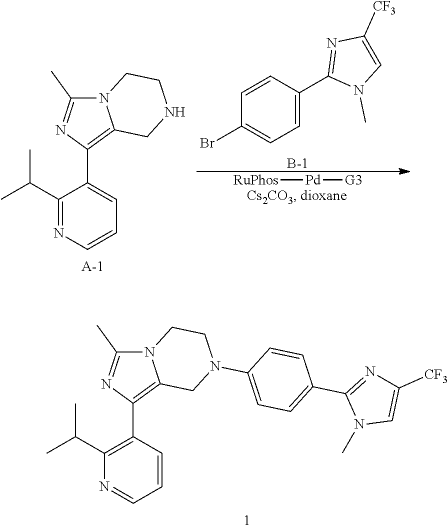

Into a 50 mL round-bottom flask, purged and maintained with an inert atmosphere of nitrogen, was placed Intermediate A-1 (200 mg, 0.78 mmol, 1.00 equiv), Intermediate B-1 (260 mg, 0.85 mmol, 1.10 equiv), cesium carbonate (509 mg, 1.56 mmol, 2.00 equiv), RuPhos-Pd-G3 (65 mg, 0.08 mmol, 0.10 equiv) and 1,4-dioxane (10 mL). The resulting mixture was stirred overnight at 110° C. After cooling to rt, the reaction mixture was filtered and concentrated under vacuum. The residue was purified by silica gel chromatography (eluting with 1/20 MeOH/DCM) and further purified by prep-HPLC (Column: XBridge BEH Shield RP18 OBD Prep Column, 130 Å, 5 μm, 19 mm×150 mm; Mobile phase: water (10 mmol NH₄HCO₃), MeCN (10% MeCN up to 40% over 7 min); Flow rate: 20 mL/min; Detector: 254 & 220 nm). This resulted in 36.1 mg (10%) of 1-(2-isopropylpyridin-3-yl)-3-methyl-7-(4-(1-methyl-4-(trifluoromethyl)-1H-imidazol-2-yl)phenyl)-5,6,7,8-tetrahydroimidazo[1,5-a]pyrazine as a white solid. ¹H NMR (300 MHz, CD₃OD) δ 8.52-8.50 (m, 1H), 7.68-7.65 (m, 1H), 7.61 (s, 1H), 7.52 (d, J=9.00 Hz, 2H), 7.32-7.28 (m, 1H), 7.09 (d, J=9.00 Hz, 2H), 4.39 (s, 2H), 4.16-4.12 (m, 2H), 3.92-3.89 (m, 2H), 3.74 (s, 3H), 3.40-3.31 (m, 1H), 2.41 (s, 3H), 1.22 (d, J=6.9 Hz, 6H). LCMS (ESI, m z) 481.1 [M+H]⁺.

### Compound 15. 2-(difluoromethoxy)-3-(3-methyl-7-{4-[1-methyl-4-(trifluoromethyl)-1H-imidazol-2-yl]phenyl}-5H,6H,7H,8H-imidazo[1,5-a]pyrazin-1-yl)pyridine

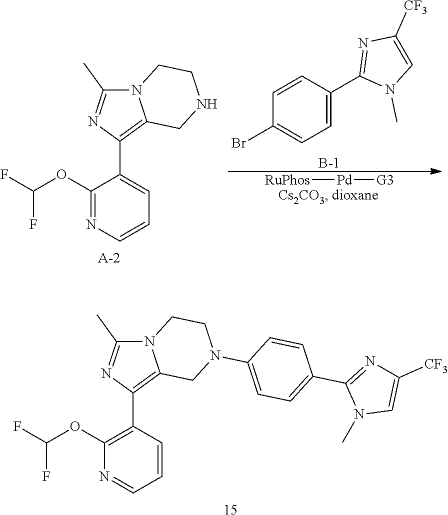

A mixture of Intermediate A-2 (100 mg, 0.36 mmol), Intermediate B-1 (109 mg, 0.36 mmol), RuPhos-Pd-G3 (30 mg, 0.04 mmol), and Cs₂CO₃ (233 mg, 0.72 mmol) in dioxane (10 mL) was stirred for 18 h at 110° C. in an oil bath. The mixture was cooled to room temperature, poured into water (20 mL) and extracted with ethyl acetate (3×20 mL). The combined organic layers were dried over anhydrous sodium sulfate, filtered, and concentrated under vacuum. The crude product was purified by Prep-HPLC with following conditions: Column: XBridge Prep Phenyl OBD Column 19×150 mm, 5 um; Mobile Phase A: water (10 mmol/L NH₄HCO₃), Mobile Phase B: ACN; Flow rate: 20 mL/min; Gradient: 25% B to 75% B in 7 min; 254 nm. The product fraction was lyophilized to afford 2-(difluoromethoxy)-3-(3-methyl-7-{4-[1-methyl-4-(trifluoromethyl)-1H-imidazol-2-yl]phenyl}-5H,6H,7H,8H-imidazo[1,5-a]pyrazin-1-yl)pyridine (21.4 mg, 12%) as a white solid. ¹H NMR (400 MHz, Methanol-d₄) δ 8.19-8.17 (m, 1H), 8.03-8.01 (m, 1H), 7.65 (s, 1H), 7.62 (d, J=1.2 Hz, 1H), 7.53 (d, J=8.8 Hz, 2H), 7.30-727 (m, 1H), 7.14 (d, J=8.8 Hz, 2H), 4.57 (s, 2H), 4.14-4.09 (m, 2H), 3.92-3.90 (m, 2H), 3.75 (s, 3H), 2.41 (s, 3H). LCMS (ES, m z): 505 [M+H]⁺.

TABLE 4

| Cmpd. No. | Purification Method |
|---|---|
| 8-1 and | The two enantiomers were separated by |
| 8-2 | CHIRAL-HPLC (Column: CHIRAL ART |
| | Cellulose-SB, 20 × 250 mm, 5 μm; Mobile |
| | Phase A: hexane-HPLC, Mobile Phase B: |
| | EtOH-HPLC; Flow rate: 20 mL/min; |
| | Gradient: 20 B to 20 B over 14 min; |
| | Detector: 220/254 nm; Retention time: |
| | 1^{st}: 9.60 min, 2^{nd}: 10.82 min). |
| 27-1 and | The two enantiomers were separated by |
| 27-2 | CHIRAL-HPLC (Column: Daicel |
| | CHIRALPAK IF, 20 × 250 mm, |
| | 5 μm; Mobile Phase A: hexane:DCM = |
| | 5:1, Mobile Phase B: EtOH-HPLC; Flow |
| | rate: 18 mL/min; Gradient: 40 B to 40 B |
| | over 10 min; Detector: 220/254 nm; Retention |
| | time: 1^{st}: 5.591 min, 2^{nd}: 6.341 min). |
| 52-1 and | The two enantiomers were separated by |
| 52-2 | CHIRAL-HPLC (Column: Daicel |
| | CHIRALPAK IE, 20 × 250 mm, |
| | 5 μm; Mobile Phase A: Hexane-HPLC, |
| | Mobile Phase B: EtOH-HPLC; Flow rate: |
| | 20 mL/min; Gradient: 30 B to 30 B over 24 min; |
| | Detector: 220/254 nm; Retention time: 1^{st}: |
| | 12.86 min, 2^{nd}: 17.16 min). |
| 72-1 and | The two enantiomers were separate |
| 72-2 | by CHIRAL-HPLC (Column: Daicel |
| | CHIRALPAK AD-H, 20 × 250 mm, |
| | 5 μm; Mobile Phase A: Hexane- |
| | HPLC, Mobile Phase B: iPrOH-HPLC; |
| | Flow rate: 20 mL/min; Gradient: |
| | 10 B to 10 B over 45 min; Detector: 220/254 |
| | nm; Retention time: 1^{st}: 20.317 min, 2^{nd}: 29.48 min). |

### Example 1. Biochemical Assay for USP1 Inhibition

The assay was performed in a final volume of 6 μL in assay buffer containing 20 mM Tris-HCl pH 8.0, (1 M Tris-HCl, pH 8.0 solution; Corning 46-031-CM), 1 mM GSH (L-glutathione reduced, Sigma-Aldrich, G4251-100G), 0.03% BGG (0.22 μM filtered, Sigma, G7516-25G), and 0.01% Triton X-100 (Sigma, T9284-10L). Nanoliter quantities of 10-point, 3-fold serial dilution in DMSO were pre-dispensed into 1536 assay plates (Corning, #3724BC) for a final test concentration of 25 μM to 1.3 nM, top to lowest dose, respectively. Concentration and incubation times were optimized for the maximal signal-to-background while maintaining initial velocity conditions at a fixed substrate concentration (<<Km). 3 μL of 2× Enzyme was added to assay plates (pre-stamped with compound), preincubated for 30 min, and then treated with 3 μL of 2× Substrate (Ub-Rho 110, UbiQ-126). Final concentrations of Enzyme and Substrate are 0.025 nM and 25 nM, respectively. Plates were read using a kinetic protocol continuously for 11 min on the Envision (Perkin Elmer) or PheraSTAR (BMG), excitation at 485 nm and emission at 535 nm or excitation at 520 nm and emission at 590 nm. For all assay formats, data were reported as percent inhibition compared with control wells based on the following equation: % inh=100*((FLU−AveLow)/(AveHigh−AveLow)), where FLU=measured Fluorescence, AveLow=average Fluorescence of no enzyme control (n=32), and AveHigh=average Fluorescence of DMSO control (n=32). IC₅₀ values were determined by curve fitting of the standard 4 parameter logistic fitting algorithm included in the Activity Base software package: TDBS XE Designer Model205. Data is fitted using the Levenburg Marquardt algorithm.

In Table 5, absolute stereochemistry has not been determined for some Compounds. Accordingly, assignment ofany Compounds as the “R” or “S” stereoisomer is arbitrary, unless otherwise noted. In some cases, Compounds are labeled with “1^{st} eluting isomer”, “2^{nd} eluting isomer”, etc. based on the purification method used to separate the stereoisomers.

As set forth in Tables 5 and 6 below, IC₅₀ values are defined as follows: ≤1 μM (+++); >1 μM and ≤10 μM (++); >10 μM and ≤25 μM (+); and >25 μM (−)

TABLE 5

| Cmpd. | IC₅₀ | | LCMS m/z | |
|---|---|---|---|---|
| No. | (μm) | Structure and Name | [M + H]⁺ | ¹H NMR |
| 1 | (+++) | 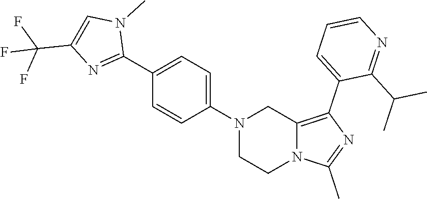 [SMILES: Cc1nc(-c2cccnc2C(C)C)c2n1CCN(c1ccc(-c3nc(C(F)(F)F)cn3C)cc1)C2] | 481.1 | (300 MHz, CD₃OD) δ 8.52- 8.50 (m, 1H), 7.68-7.65 (m, 1H), 7.61 (s, 1H), 7.52 (d, J = 9.00 Hz, 2H), 7.32-7.28 (m, 1H), 7.09 (d, J = 9.00 Hz, 2H), 4.39 (s, 2H), 4.16-4.12 (m, 2H), 3.92-3.89 (m, 2H), 3.74 (s, 3H), 3.40-3.31 (m, 1H), 2.41 (s, 3H), 1.22 (d, J = 6.9 Hz, 6H) |
| | | 3-(3-methyl-7-{4-[1-methyl-4-(trifluoromethyl)- | | |
| | | 1H-imidazol-2-yl]phenyl}-5H,6H,7H,8H- | | |
| | | imidazo[1,5-a]pyrazin-1-yl)-2-(propan-2- | | |
| | | yl)pyridine | | |
| 2 | (+++) | 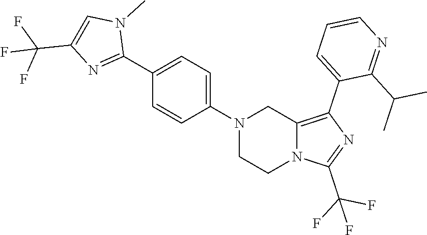 [SMILES: CC(C)c1ncccc1-c1nc(C(F)(F)F)n2c1CN(c1ccc(-c3nc(C(F)(F)F)cn3C)cc1)CC2] | 535.1 | (400 MHz, CD₃OD) δ 8.59- 8.56 (m, 1H), 7.74-7.72 (m, 1H), 7.65-7.63 (m, 1H), 7.59- 7.50 (m, 2H), 7.35-7.28 (m, 1H), 7.17-7.08 (m, 2H), 4.51- 4.39 (m, 4H), 3.95-3.92 (m, 2H), 3.74 (s, 3H), 3.27-3.24 (m, 1H), 1.24 (d, J = 6.80 Hz, 6H). |
| | | 3-(7-{4-[1-methyl-4-(trifluoromethyl)-1H- | | |
| | | imidazol-2-yl]phenyl}-3-(trifluoromethyl)- | | |
| | | 5H,6H,7H,8H-imidazo[1,5-a]pyrazin-1-yl)-2- | | |
| | | (propan-2-yl)pyridine | | |
| 3 | (+++) | 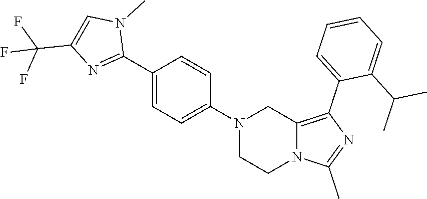 [SMILES: Cc1nc(-c2ccccc2C(C)C)c2n1CCN(c1ccc(-c3nc(C(F)(F)F)cn3C)cc1)C2] | 480.1 | (400 MHz, CD₃OD) δ 7.62 (d, J = 1.20 Hz, 1H), 7.56- 7.47 (m, 2H), 7.45-7.30 (m, 2H), 7.26-7.15 (m, 2H), 7.11- 7.03 (m, 2H), 4.36 (s, 2H), 4.12 (t, J = 5.60 Hz, 2H), 3.93-3.85 (m, 2H), 3.73 (s, 3H), 3.22-3.16 (m, 1H), 2.39 (s, 3H), 1.16 (d, J = 6.80 Hz, 6H). |
| | | 1-methyl-2-(4-{3-methyl-1-[2-(propan-2- | | |
| | | yl)phenyl]-5H,6H,7H,8H-imidazo[1,5-a]pyrazin- | | |
| | | 7-yl}phenyl)-4-(trifluoromethyl)-1H-imidazole | | |
| 4 | (+++) | 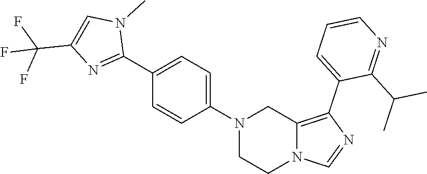 [SMILES: CC(C)c1ncccc1-c1ncn2c1CN(c1ccc(-c3nc(C(F)(F)F)cn3C)cc1)CC2] | 467 | (CD₃OD, 400 MHz) δ (ppm): 8.53-8.52 (m, 1H), 7.80 (s, 1H), 7.76-7.69 (m, 1H), 7.65-7.63 (m, 1H), 7.59- 7.51 (m, 2H), 7.38-7.30 (m, 1H), 7.16-7.09 (m, 2H), 4.47 (s, 2H), 4.39-4.32 (m, 2H), 3.96-3.88 (m, 2H), 3.77 (s, 3H), 3.43-3.33 (m, 1H), 1.25 (d, J = 6.8 Hz, 6H). |
| | | 3-(7-{4-[1-methyl-4-(trifluoromethyl)-1H- | | |
| | | imidazol-2-yl]phenyl}-5H,6H,7H,8H- | | |
| | | imidazo[1,5-a]pyrazin-1-yl)-2-(propan-2- | | |
| | | yl)pyridine | | |
| 5 | (−) | 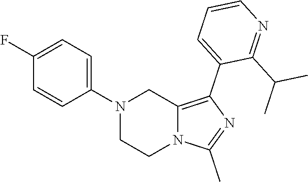 [SMILES: Cc1nc(-c2cccnc2C(C)C)c2n1CCN(c1ccc(F)cc1)C2] | 351.3 | |
| | | 3-[7-(4-fluorophenyl)-3-methyl-5H,6H,7H,8H- | | |
| | | imidazo[1,5-a]pyrazin-1-yl]-2-(propan-2- | | |
| | | yl)pyridine | | |
| 6 | (−) | 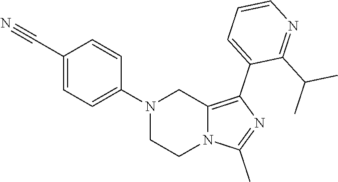 [SMILES: Cc1nc(-c2cccnc2C(C)C)c2n1CCN(c1ccc(C#N)cc1)C2] | 358.3 | |
| | | 4-{3-methyl-1-[2-(propan-2-yl)pyridin-3-yl]- | | |
| | | 5H,6H,7H,8H-imidazo[1,5-a]pyrazin-7- | | |
| | | yl}benzonitrile | | |
| 7 | (+++) | 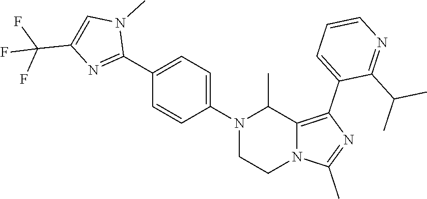 [SMILES: Cc1nc(-c2cccnc2C(C)C)c2n1CCN(c1ccc(-c3nc(C(F)(F)F)cn3C)cc1)C2C] | | |
| | | 3-(3,8-dimethyl-7-{4-[1-methyl-4- | | |
| | | (trifluoromethyl)-1H-imidazol-2-yl]phenyl}- | | |
| | | 5H,6H,7H,8H-imidazo[1,5-a]pyrazin-1-yl)-2- | | |
| | | (propan-2-yl)pyridine | | |
| 8-1 1^{st} eluting isomer 8-2 2^{nd} eluting isomer | (+++) (+++) | 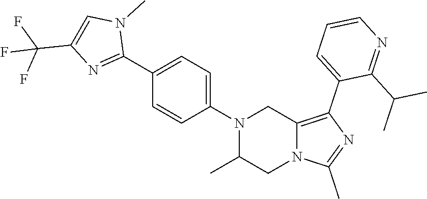 [SMILES: Cc1nc(-c2cccnc2C(C)C)c2n1CC(C)N(c1ccc(-c3nc(C(F)(F)F)cn3C)cc1)C2] | 495.4 | (400 MHz, CD₃OD) δ 8.53- 8.51 (m, 1H), 7.72-7.69 (m, 1H), 7.61 (s, 1H), 7.53 (d, J = 8.80 Hz, 2H), 7.32-7.30 (m, 1H), 7.03 (d, J = 8.80 Hz, 2H), 4.65-4.62 (m, 1H), 4.49 (d, J = 14.80 Hz, 1H), 4.24- 4.14 (m, 3H), 3.74 (s, 3H), 3.38-3.31 (m, 1H), 2.45 (s, 3H), 1.23-1.17 (m, 9H). |
| | | 3-[3,6-dimethyl-7-{4-[1-methyl-4- | | |
| | | (trifluoromethyl)-1H-imidazol-2-yl]phenyl}- | | |
| | | 5H,6H,7H,8H-imidazo[1,5-a]pyrazin-1-yl]-2- | | |
| | | (propan-2-yl)pyridine | | |
| 9 | (++) | 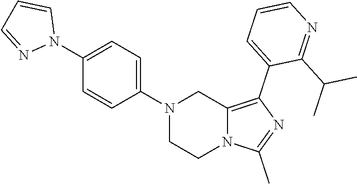 [SMILES: Cc1nc(-c2cccnc2C(C)C)c2n1CCN(c1ccc(-n3cccn3)cc1)C2] | 399.3 | |
| | | 3-{3-methyl-7-[4-(1H-pyrazol-1-yl)phenyl]- | | |
| | | 5H,6H,7H,8H-imidazo[1,5-a]pyrazin-1-yl}-2- | | |
| | | (propan-2-yl)pyridine | | |
| 10 | (−) | 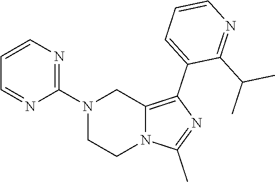 [SMILES: Cc1nc(-c2cccnc2C(C)C)c2n1CCN(c1ncccn1)C2] | 335.4 | |
| | | 2-{3-methyl-1-[2-(propan-2-yl)pyridin-3-yl]- | | |
| | | 5H,6H,7H,8H-imidazo[1,5-a]pyrazin-7- | | |
| | | yl}pyrimidine | | |
| 11 | (−) | 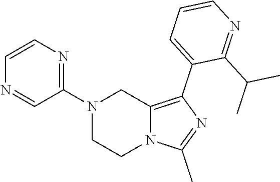 [SMILES: Cc1nc(-c2cccnc2C(C)C)c2n1CCN(c1cnccn1)C2] | 335.4 | |
| | | 2-{3-methyl-1-[2-(propan-2-yl)pyridin-3-yl]- | | |
| | | 5H,6H,7H,8H-imidazo[1,5-a]pyrazin-7- | | |
| | | yl}pyrazine | | |
| 12 | (−) | 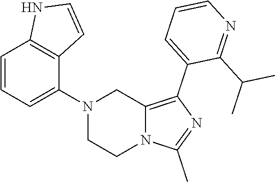 [SMILES: Cc1nc(-c2cccnc2C(C)C)c2n1CCN(c1cccc3[nH]ccc13)C2] | 372.4 | |
| | | 4-{3-methyl-1-[2-(propan-2-yl)pyridin-3-yl]- | | |
| | | 5H,6H,7H,8H-imidazo[1,5-a]pyrazin-7-yl}-1H- | | |
| | | indole | | |
| 13 | (+++) | 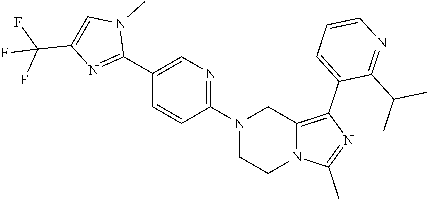 [SMILES: Cc1nc(-c2cccnc2C(C)C)c2n1CCN(c1ccc(-c3nc(C(F)(F)F)cn3C)cn1)C2] | 482.2 | (400 MHz, CD₃OD) δ 8.52- 8.50 (m, 1H), 7.40-8.39 (m, 1H), 7.85-7.82 (m, 1H), 7.68- 7.66 (m, 2H), 7.32-7.29 (m, 1H), 7.00-6.98 (m, 1H), 4.74 (s, 2H), 4.18-4.14 (m, 4H), 3.75 (s, 3H), 3.39-3.32 (m, 1H), 2.41 (s, 3H), 1.21 (d, J = 6.80 Hz, 6H). |
| | | 2-{3-methyl-1-[2-(propan-2-yl)pyridin-3-yl]- | | |
| | | 5H,6H,7H,8H-imidazo[1,5-a]pyrazin-7-yl}-5-[1- | | |
| | | methyl-4-(trifluoromethyl)-1H-imidazol-2- | | |
| | | yl]pyridine | | |
| 14 | (++) | 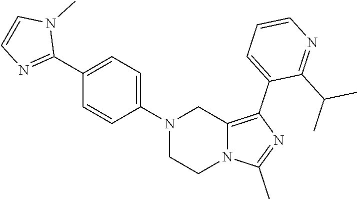 [SMILES: Cc1nc(-c2cccnc2C(C)C)c2n1CCN(c1ccc(-c3nccn3C)cc1)C2] | 413.4 | |
| | | 3-{3-methyl-7-[4-(1-methyl-1H-imidazol-2- | | |
| | | yl)phenyl]-5H,6H,7H,8H-imidazo[1,5-a]pyrazin- | | |
| | | 1-yl}-2-(propan-2-yl)pyridine | | |
| 15 | (+++) | 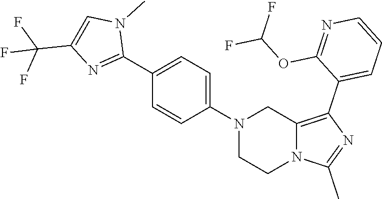 [SMILES: Cc1nc(-c2cccnc2OC(F)F)c2n1CCN(c1ccc(-c3nc(C(F)(F)F)cn3C)cc1)C2] | 505.0 | (400 MHz, CD₃OD) δ 8.24- 8.12 (m, 1H), 8.08-7.98 (m, 1H), 7.65-7.49 (m, 4H), 7.35- 7.20(m, 1H), 7.19-7.10 (m, 2H), 4.57 (s, 2H), 4.20-4.05 (m, 2H), 3.98-3.80(m, 2H), 3.75 (s, 3H, 2.41 (s, 3H). |
| | | 2-(difluoromethoxy)-3-(3-methyl-7-{4-[1- | | |
| | | methyl-4-(trifluoromethyl)-1H-imidazol-2- | | |
| | | yl]phenyl}-5H,6H,7H,8H-imidazo[1,5-a]pyrazin- | | |
| | | 1-yl)pyridine | | |
| 16 | (+++) | 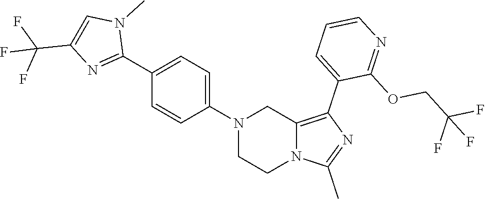 [SMILES: Cc1nc(-c2cccnc2OCC(F)(F)F)c2n1CCN(c1ccc(-c3nc(C(F)(F)F)cn3C)cc1)C2] | 537.0 | (400 MHz, CD₃OD) δ 8.18- 8.11 (m, 1H), 7.97-7.88(m, 1H), 7.65-7.60 (m, 1H), 7.56- 7.48 (m, 2H), 7.20-7.07 (m, 3H), 5.98-4.98 (m, 2H), 4.52 (s, 2H), 4.18-4.05 (m, 2H), 3.93-3.84(m, 2H), 3.74(s, 3H), 2.40 (s, 3H). |
| | | 3-(3-methyl-7-{4-[1-methyl-4-(trifluoromethyl)- | | |
| | | 1H-imidazol-2-yl]phenyl}-5H,6H,7H,8H- | | |
| | | imidazo[1,5-a)pyrazin-1-yl)-2-(2,2,2- | | |
| | | trifluoroethoxy)pyridine | | |
| 17 | (−) | 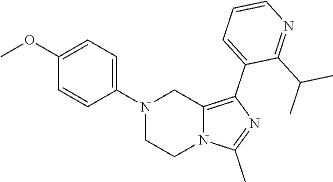 [SMILES: COc1ccc(N2CCn3c(C)nc(-c4cccnc4C(C)C)c3C2)cc1] | 363.4 | |
| | | 3-[7-(4-methoxyphenyl)-3-methyl-5H,6H,7H,8H- | | |
| | | imidazo[1,5-a]pyrazin-1-yl]-2-(propan-2- | | |
| | | yl)pyridine | | |
| 18 | (−) | 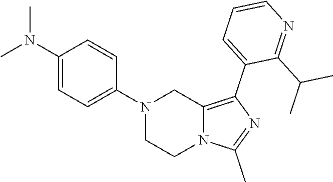 [SMILES: Cc1nc(-c2cccnc2C(C)C)c2n1CCN(c1ccc(N(C)C)cc1)C2] | 376.4 | |
| | | N,N-dimethyl-4-{3-methyl-1-[2-(propan-2- | | |
| | | yl)pyridin-3-yl]-5H,6H,7H,8H-imidazo[1,5- | | |
| | | a]pyrazin-7-yl}aniline | | |
| 19 | (+) | 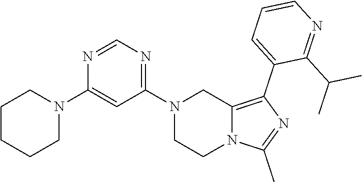 [SMILES: Cc1nc(-c2cccnc2C(C)C)c2n1CCN(c1cc(N3CCCCC3)ncn1)C2] | 418.4 | |
| | | 4-{3-methyl-1-[2-(propan-2-yl)pyridin-3-yl]- | | |
| | | 5H,6H,7H,8H-imidazo[1,5-a]pyrazin-7-yl}-6- | | |
| | | (piperidin-1-yl)pyrimidine | | |
| 20 | (+++) | 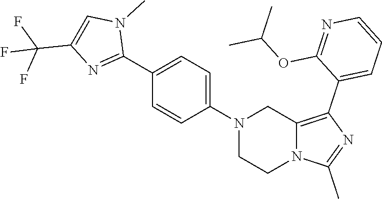 [SMILES: Cc1nc(-c2cccnc2OC(C)C)c2n1CCN(c1ccc(-c3nc(C(F)(F)F)cn3C)cc1)C2] | 497.1 | (400 MHz, CD₃OD) δ 8.17- 8.05(m, 1H), 7.90-7.90 (m, 1H), 7.65-7.48(m, 1H), 7.56- 7.48 (m, 2H), 7.20-7.07 (m, 3H), 5.51-5.39 (m, 1H), 4.52(s, 2H), 4.20-4.05 (m, 2H), 3.95-3.81 (m, 2H), 3.31 (s, 3H), 2.40 (s, 3H), 1.45- 1.31(m, 6H). |
| | | 3-(3-methyl-7-{4-[1-methyl-4-(trifluoromethyl)- | | |
| | | 1H-imidazol-2-yl]phenyl}-5H,6H,7H,8H- | | |
| | | imidazo[1,5-a]pyrazin-1-yl)-2-(propan-2- | | |
| | | yloxy)pyridine | | |
| 21 | (+) | 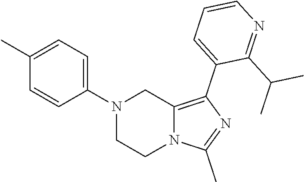 [SMILES: Cc1ccc(N2CCn3c(C)nc(-c4cccnc4C(C)C)c3C2)cc1] | 347.5 | |
| | | 3-[3-methyl-7-(4-methylphenyl)-5H,6H,7H,8H- | | |
| | | imidazo[1,5-a]pyrazin-1-yl]-2-(propan-2- | | |
| | | yl)pyridine | | |
| 22 | (+) | 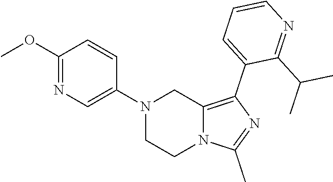 [SMILES: COc1ccc(N2CCn3c(C)nc(-c4cccnc4C(C)C)c3C2)cn1] | 364.4 | |
| | | 2-methoxy-5-{3-methyl-1-[2-(propan-2- | | |
| | | yl)pyridin-3-yl]-5H,6H,7H,8H-imidazo[1,5- | | |
| | | a]pyrazin-7-yl}pyridine | | |
| 23 | (+) | 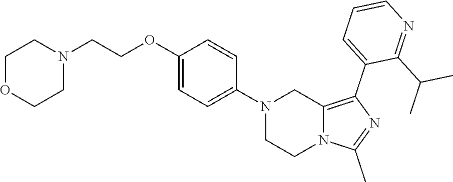 [SMILES: Cc1nc(-c2cccnc2C(C)C)c2n1CCN(c1ccc(OCCN3CCOCC3)cc1)C2] | 462.5 | |
| | | 4-[2-(4-{3-methyl-1-[2-(propan-2-yl)pyridin-3- | | |
| | | yl]-5H,6H,7H,8H-imidazo[1,5-a]pyrazin-7- | | |
| | | yl}phenoxy)ethyl]morpholine | | |
| 24 | (+++) | 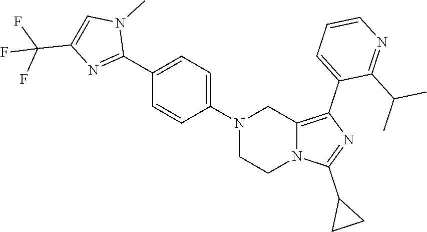 [SMILES: CC(C)c1ncccc1-c1nc(C2CC2)n2c1CN(c1ccc(-c3nc(C(F)(F)F)cn3C)cc1)CC2] | 507.1 | (400 MHz, CD₃OD) δ 8.53- 8.52 (m, 1H), 7.69-7.64 (m, 2H), 7.57-7.53 (m, 2H), 7.33- 7.30 (m, 1H), 7.13-7.11 (m, 2H), 4.41 (s, 2H), 4.31-4.29 (m, 2H), 3.95-3.93 (m, 2H), 3.76 (s, 3H), 3.33-3.29 (m, 1H), 2.02-1.98 (m, 1H), 1.23 (d, J = 7.20 Hz, 6H), 1.06- 0.95 (m, 4H). |
| | | 3-(3-cyclopropyl-7-{4-[1-methyl-4- | | |
| | | (trifluoromethyl)-1H-imidazol-2-yl]phenyl}- | | |
| | | 5H,6H,7H,8H-imidazo[1,5-a]pyrazin-1-yl)-2- | | |
| | | (propan-2-yl)pyridine | | |
| 25 | (+++) | 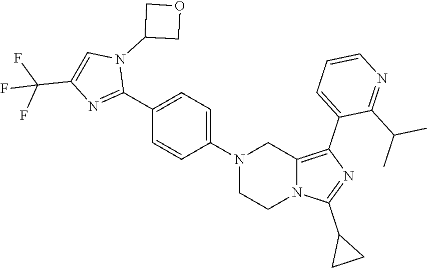 [SMILES: CC(C)c1ncccc1-c1nc(C2CC2)n2c1CN(c1ccc(-c3nc(C(F)(F)F)cn3C3COC3)cc1)CC2] | 523 | (CD₃OD, 400 MHz) δ (ppm): 8.57-8.55 (m, 1H), 8.22 (s, 1H), 7.73-7.70 (m, 1H), 7.42-7.33 (m, 3H), 7.12 (d, J = 8.8 Hz, 2H), 5.57-5.51 (m, 1H), 4.99 (t, J = 7.2 Hz, 2H), 4.87-4.83 (m, 2H), 4.44 (s, 2H), 4.21-4.18 (m, 2H), 3.96-3.94 (m, 2H), 3.38-3.34 (m, 1H), 2.47 (s, 3H), 1.25 (d, J = 6.8 Hz, 6H). |
| | | 3-(3-methyl-7-{4-[1-(oxetan-3-yl)-4- | | |
| | | (trifluoromethyl)-1H-imidazol-2-yl]phenyl}- | | |
| | | 5H,6H,7H,8H-imidazo[1,5-a]pyrazin-1-yl)-2- | | |
| | | (propan-2-yl)pyridine | | |
| 26 | (−) | 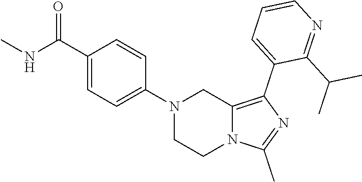 [SMILES: CNC(=O)c1ccc(N2CCn3c(C)nc(-c4cccnc4C(C)C)c3C2)cc1] | 390.4 | |
| | | N-methyl-4-{3-methyl-1-[2-(propan-2-yl)pyridin- | | |
| | | 3-yl]-5H,6H,7H,8H-imidazo[1,5-a]pyrazin-7- | | |
| | | yl}benzamide | | |
| 27-1 1^{st} eluting isomer 27-2 2^{nd} eluting isomer | (+++) (+++) | 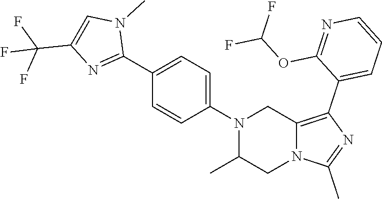 [SMILES: Cc1nc(-c2cccnc2OC(F)F)c2n1CC(C)N(c1ccc(-c3nc(C(F)(F)F)cn3C)cc1)C2] | 519 | ¹H-NMR (CD₃OD, 400 MHz) δ (ppm): 8.22-8.19 (m, 1H), 8.19-8.05 (m, 1H), 7.76 (t, J = 73.2 Hz, 1H), 7.65- 7.52 (m, 3H), 7.21-7.17 (m, 1H), 7.11-7.05 (m, 2H), 4.70- 4.60 (m, 2H), 4.40-4.36 (m, 1H), 4.21-4.11 (m, 2H), 3.76 (s, 3H), 2.45 (s, 3H), 1.18 (d, J = 6.4 Hz, 3H). |
| | | 2-(difluoromethoxy)-3-[-3,6-dimethyl-7-{4-[1- | | |
| | | methyl-4-(trifluoromethyl)-1H-imidazol-2- | | |
| | | yl]phenyl}-5H,6H,7H,8H-imidazo[1,5-a]pyrazin- | | |
| | | 1-yl]pyridine | | |
| 28 | (−) | 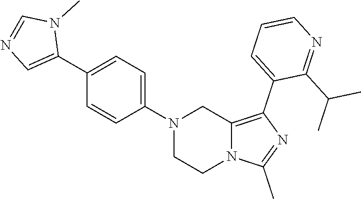 [SMILES: Cc1nc(-c2cccnc2C(C)C)c2n1CCN(c1ccc(-c3cncn3C)cc1)C2] | 413.5 | |
| | | 3-{3-methyl-7-[4-(1-methyl-1H-imidazol-5- | | |
| | | yl)phenyl]-5H,6H,7H,8H-imidazo[1,5-a]pyrazin- | | |
| | | 1-yl}-2-(propan-2-yl)pyridine | | |
| 29 | (−) | 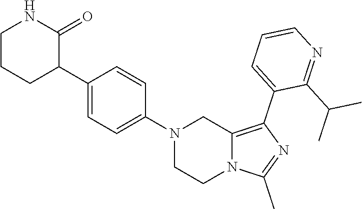 [SMILES: Cc1nc(-c2cccnc2C(C)C)c2n1CCN(c1ccc(C3CCCNC3=O)cc1)C2] | 430.5 | |
| | | 3-(4-{3-methyl-1-[2-(propan-2-yl)pyridin-3-yl]- | | |
| | | 5H,6H,7H,8H-imidazo[1,5-a]pyrazin-7- | | |
| | | yl}phenyl)piperidin-2-one | | |
| 30 | (−) | 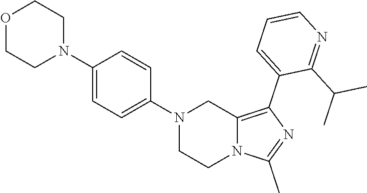 [SMILES: Cc1nc(-c2cccnc2C(C)C)c2n1CCN(c1ccc(N3CCOCC3)cc1)C2] | 418.5 | |
| | | 4-(4-{3-methyl-1-[2-(propan-2-yl)pyridin-3-yl]- | | |
| | | 5H,6H,7H,8H-imidazo[1,5-a]pyrazin-7- | | |
| | | yl}phenyl)morpholine | | |
| 31 | (+++) | 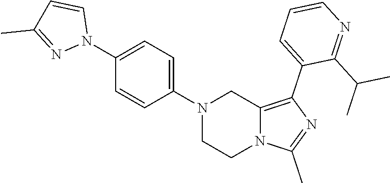 [SMILES: Cc1ccn(-c2ccc(N3CCn4c(C)nc(-c5cccnc5C(C)C)c4C3)cc2)n1] | 413.5 | |
| | | 3-{3-methyl-7-[4-(3-methyl-1H-pyrazol-1- | | |
| | | yl)phenyl]-5H,6H,7H,8H-imidazo[1,5-a]pyrazin- | | |
| | | 1-yl}-2-(propan-2-yl)pyridine | | |
| 32 | (++) | 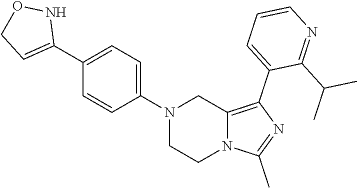 [SMILES: Cc1nc(-c2cccnc2C(C)C)c2n1CCN(c1ccc(C3=CCON3)cc1)C2] | 402.4 | |
| | | 3-{7-[4-(2,5-dihydro-1,2-oxazol-3-yl)phenyl]-3- | | |
| | | methyl-5H,6H,7H,8H-imidazo[1,5-a]pyrazin-1- | | |
| | | yl}-2-(propan-2-yl)pyridine | | |
| 33 | (+) | 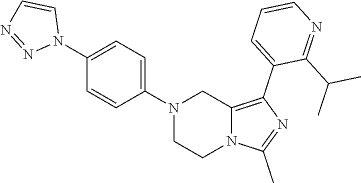 [SMILES: Cc1nc(-c2cccnc2C(C)C)c2n1CCN(c1ccc(-n3ccnn3)cc1)C2] | 400.4 | |
| | | 3-{3-methyl-7-[4-(1H-1,2,3-triazol-1-yl)phenyl]- | | |
| | | 5H,6H,7H,8H-imidazo[1,5-a]pyrazin-1-yl}-2- | | |
| | | (propan-2-yl)pyridine | | |
| 34 | (−) | 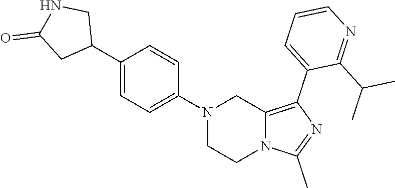 [SMILES: Cc1nc(-c2cccnc2C(C)C)c2n1CCN(c1ccc(C3CNC(=O)C3)cc1)C2] | 416.5 | |
| | | 4-(4-{3-methyl-1-[2-(propan-2-yl)pyridin-3-yl]- | | |
| | | 5H,6H,7H,8H-imidazo[1,5-a]pyrazin-7- | | |
| | | yl}phenyl)pyrrolidin-2-one | | |
| 35 | (−) | 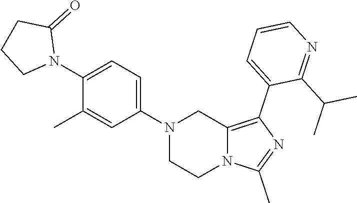 [SMILES: Cc1cc(N2CCn3c(C)nc(-c4cccnc4C(C)C)c3C2)ccc1N1CCCC1=O] | 430.5 | |
| | | 1-(2-methyl-4-{3-methyl-1-[2-(propan-2- | | |
| | | yl)pyridin-3-yl]-5H,6H,7H,8H-imidazo[1,5- | | |
| | | a]pyrazin-7-yl}phenyl)pyrrolidin-2-one | | |
| 36 | (+) | 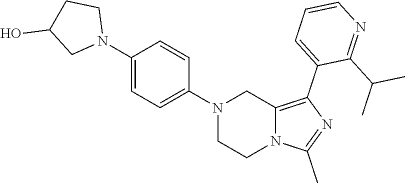 [SMILES: Cc1nc(-c2cccnc2C(C)C)c2n1CCN(c1ccc(N3CCC(O)C3)cc1)C2] | 418.5 | |
| | | 1-(4-{3-methyl-1-[2-(propan-2-yl)pyridin-3-yl]- | | |
| | | 5H,6H,7H,8H-imidazo[1,5-a]pyrazin-7- | | |
| | | yl}phenyl)pyrrolidin-3-ol | | |
| 37 | (−) | 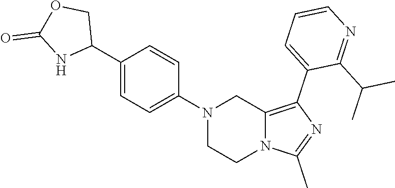 [SMILES: Cc1nc(-c2cccnc2C(C)C)c2n1CCN(c1ccc(C3COC(=O)N3)cc1)C2] | 418.4 | |
| | | 4-(4-{3-methyl-1-[2-(propan-2-yl)pyridin-3-yl]- | | |
| | | 5H,6H,7H,8H-imidazo[1,5-a]pyrazin-7- | | |
| | | yl}phenyl)-1,3-oxazolidin-2-one | | |
| 38 | (−) | 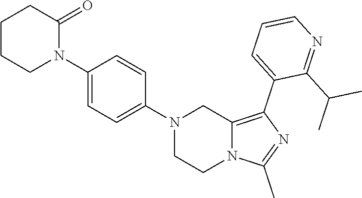 [SMILES: Cc1nc(-c2cccnc2C(C)C)c2n1CCN(c1ccc(N3CCCCC3=O)cc1)C2] | 430.5 | |
| | | 1-(4-{3-methyl-1-[2-(propan-2-yl)pyridin-3-yl]- | | |
| | | 5H,6H,7H,8H-imidazo[1,5-a]pyrazin-7- | | |
| | | yl}phenyl)piperidin-2-one | | |
| 39 | (−) | 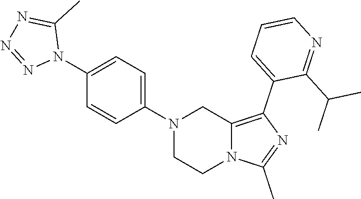 [SMILES: Cc1nnnn1-c1ccc(N2CCn3c(C)nc(-c4cccnc4C(C)C)c3C2)cc1] | 415.5 | |
| | | 3-{3-methyl-7-[4-(5-methyl-1H-1,2,3,4-tetrazol- | | |
| | | 1-yl)phenyl]-5H,6H,7H,8H-imidazo[1,5- | | |
| | | a]pyrazin-1-yl}-2-(propan-2-yl)pyridine | | |
| 40 | (++) | 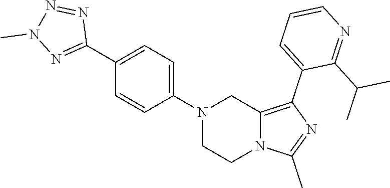 [SMILES: Cc1nc(-c2cccnc2C(C)C)c2n1CCN(c1ccc(-c3nnn(C)n3)cc1)C2] | 415.5 | |
| | | 3-{3-methyl-7-[4-(2-methyl-2H-1,2,3,4-tetrazol- | | |
| | | 5-yl)phenyl]-5H,6H,7H,8H-imidazo[1,5- | | |
| | | a]pyrazin-1-yl}-2-(propan-2-yl)pyridine | | |
| 41 | (++) | 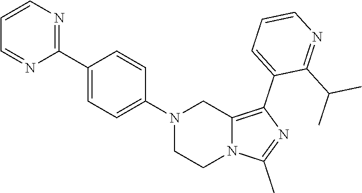 [SMILES: Cc1nc(-c2cccnc2C(C)C)c2n1CCN(c1ccc(-c3ncccn3)cc1)C2] | 411.5 | |
| | | 2-(4-{3-methyl-1-[2-(propan-2-yl)pyridin-3-yl]- | | |
| | | 5H,6H,7H,8H-imidazo[1,5-a]pyrazin-7- | | |
| | | yl}phenyl)pyrimidine | | |
| 42 | (−) | 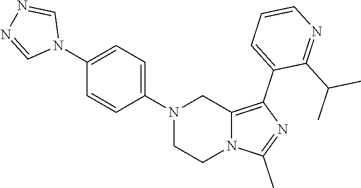 [SMILES: Cc1nc(-c2cccnc2C(C)C)c2n1CCN(c1ccc(-n3cnnc3)cc1)C2] | 400.5 | |
| | | 3-{3-methyl-7-[4-(4H-1,2,4-triazol-4-yl)phenyl]- | | |
| | | 5H,6H,7H,8H-imidazo[1,5-a]pyrazin-1-yl}-2- | | |
| | | (propan-2-yl)pyridine | | |
| 43 | (++) | 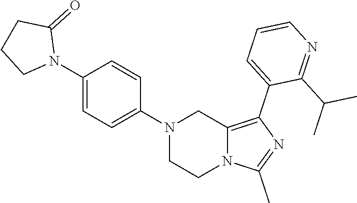 [SMILES: Cc1nc(-c2cccnc2C(C)C)c2n1CCN(c1ccc(N3CCCC3=O)cc1)C2] | 416.5 | |
| | | 1-(4-{3-methyl-1-[2-(propan-2-yl)pyridin-3-yl]- | | |
| | | 5H,6H,7H,8H-imidazo[1,5-a]pyrazin-7- | | |
| | | yl}phenyl)pyrrolidin-2-one | | |
| 44 | (++) | 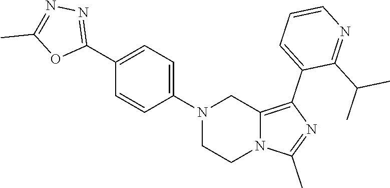 [SMILES: Cc1nnc(-c2ccc(N3CCn4c(C)nc(-c5cccnc5C(C)C)c4C3)cc2)o1] | 415.5 | |
| | | 3-{3-methyl-7-[4-(5-methyl-1,3,4-oxadiazol-2- | | |
| | | yl)phenyl]-5H,6H,7H,8H-imidazo[1,5-a]pyrazin- | | |
| | | 1-yl}-2-(propan-2-yl)pyridine | | |
| 45 | (+++) | 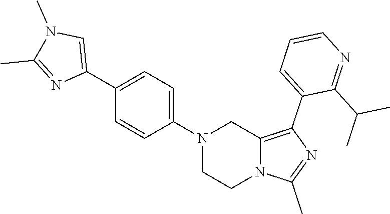 [SMILES: Cc1nc(-c2ccc(N3CCn4c(C)nc(-c5cccnc5C(C)C)c4C3)cc2)cn1C] | 427.5 | |
| | | 3-{7-[4-(1,2-dimethyl-1H-imidazol-4-yl)phenyl]- | | |
| | | 3-methyl-5H,6H,7H,8H-imidazo[1,5-a]pyrazin-1- | | |
| | | yl}-2-(propan-2-yl)pyridine | | |
| 46 | (−) | 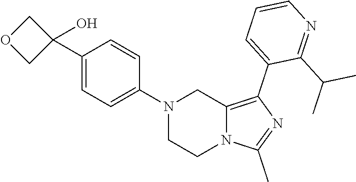 [SMILES: Cc1nc(-c2cccnc2C(C)C)c2n1CCN(c1ccc(C3(O)COC3)cc1)C2] | 405.4 | |
| | | 3-(4-{3-methyl-1-[2-(propan-2-yl)pyridin-3-yl]- | | |
| | | 5H,6H,7H,8H-imidazo[1,5-a]pyrazin-7- | | |
| | | yl}phenyl)oxetan-3-ol | | |
| 47 | (−) | 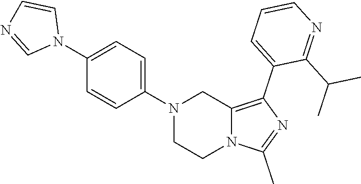 [SMILES: Cc1nc(-c2cccnc2C(C)C)c2n1CCN(c1ccc(-n3ccnc3)cc1)C2] | 399.5 | |
| | | 3-{7-[4-(1H-imidazol-1-yl)phenyl]-3-methyl- | | |
| | | 5H,6H,7H,8H-imidazo[1,5-a]pyrazin-1-yl}-2- | | |
| | | (propan-2-yl)pyridine | | |
| 48 | (−) | 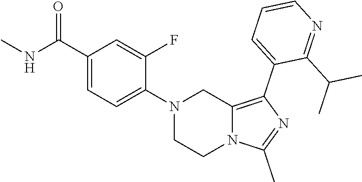 [SMILES: CNC(=O)c1ccc(N2CCn3c(C)nc(-c4cccnc4C(C)C)c3C2)c(F)c1] | 408.4 | |
| | | 3-fluoro-N-methyl-4-{3-methyl-1-[2-(propan-2- | | |
| | | yl)pyridin-3-yl]-5H,6H,7H,8H-imidazo[1,5- | | |
| | | a]pyrazin-7-yl}benzamide | | |
| 49 | (+) | 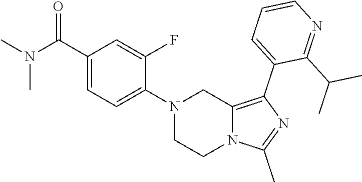 [SMILES: Cc1nc(-c2cccnc2C(C)C)c2n1CCN(c1ccc(C(=O)N(C)C)cc1F)C2] | 422.4 | |
| | | 3-fluoro-N,N-dimethyl-4-{3-methyl-1-[2- | | |
| | | (propan-2-yl)pyridin-3-yl]-5H,6H,7H,8H- | | |
| | | imidazo[1,5-a]pyrazin-7-yl}benzamide | | |
| 50 | (+) | 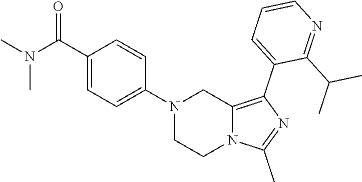 [SMILES: Cc1nc(-c2cccnc2C(C)C)c2n1CCN(c1ccc(C(=O)N(C)C)cc1)C2] | 404.5 | |
| | | N,N-dimethyl-4-{3-methyl-1-[2-(propan-2- | | |
| | | yl)pyridin-3-yl]-5H,6H,7H,8H-imidazo[1,5- | | |
| | | a]pyrazin-7-yl}benzamide | | |
| 51 | (+++) | 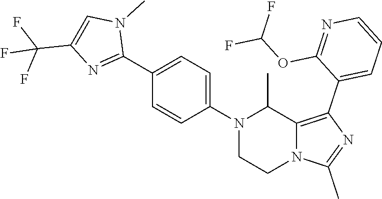 [SMILES: Cc1nc(-c2cccnc2OC(F)F)c2n1CCN(c1ccc(-c3nc(C(F)(F)F)cn3C)cc1)C2C] | 519.5 | |
| | | 2-(difluoromethoxy)-3-(3,8-dimethyl-7-{4-[1- | | |
| | | methyl-4-(trifluoromethyl)-1H-imidazol-2- | | |
| | | yl]phenyl}-5H,6H,7H,8H-imidazo[1,5-a]pyrazin- | | |
| | | 1-yl)pyridine | | |
| 51 | (−) | 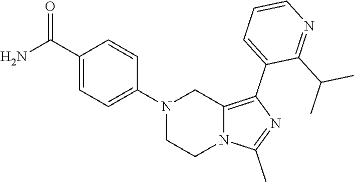 [SMILES: Cc1nc(-c2cccnc2C(C)C)c2n1CCN(c1ccc(C(N)=O)cc1)C2] | 376.4 | |
| | | 4-{3-methyl-1-[2-(propan-2-yl)pyridin-3-yl]- | | |
| | | 5H,6H,7H,8H-imidazo[1,5-a]pyrazin-7- | | |
| | | yl}benzamide | | |
| 52-1 1^{st} eluting isomer 52-2 2^{nd} eluting isomer | (+++) (++) | 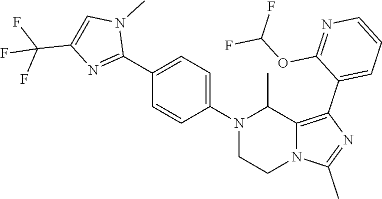 [SMILES: Cc1nc(-c2cccnc2OC(F)F)c2n1CCN(c1ccc(-c3nc(C(F)(F)F)cn3C)cc1)C2C] | 519 | (CD₃OD, 400 MHz) δ (ppm): 8.21-8.19 (m, 1H), 8.04-7.97 (m, 1H), 7.81 (t, J = 73.6 Hz, 1H), 7.62 (s, 1H), 7.56-7.53 (m, 2H), 7.31-7.27 (m, 1H), 7.19-7.16 (m, 2H), 5.66-5.61 (m, 1H), 4.14-4.09 (m, 2H), 4.01-3.99 (m, 1H), 3.77 (s, 3H), 3.68-3.61 (m, 1H), 2.41 (s, 3H), 1.17 (d, J = 6.8 Hz, 3H). |
| | | 2-(difluoromethoxy)-3-[-3,8-dimethyl-7-{4-[1- | | |
| | | methyl-4-(trifluoromethyl)-1H-imidazol-2- | | |
| | | yl]phenyl}-5H,6H,7H,8H-imidazo[1,5-a]pyrazin- | | |
| | | 1-yl]pyridine | | |
| 53 | (++) | 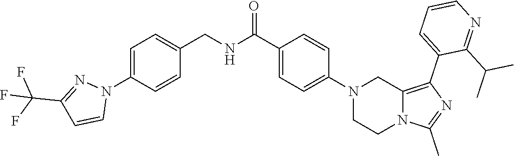 [SMILES: Cc1nc(-c2cccnc2C(C)C)c2n1CCN(c1ccc(C(=O)NCc3ccc(-n4ccc(C(F)(F)F)n4)cc3)cc1)C2] | 600.6 | |
| | | 4-{3-methyl-1-[2-(propan-2-yl)pyridin-3-yl]- | | |
| | | 5H,6H,7H,8H-imidazo[1,5-a]pyrazin-7-yl}-N- | | |
| | | ({4-[3-(trifluoromethyl)-1H-pyrazol-1- | | |
| | | yl]phenyl}methyl)benzamide | | |
| 54-1 | (+) | 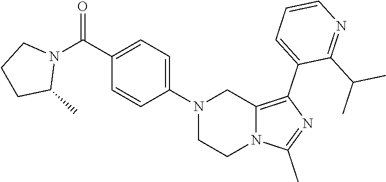 [SMILES: Cc1nc(-c2cccnc2C(C)C)c2n1CCN(c1ccc(C(=O)N3CCC[C@H]3C)cc1)C2] | 444.5 | |
| | | 3-(3-methyl-7-{4-[(2R)-2-methylpyrrolidine-1- | | |
| | | carbonyl]phenyl}-5H,6H,7H,8H-imidazo[1,5- | | |
| | | a]pyrazin-1-yl)-2-(propan-2-yl)pyridine | | |
| 55 | (++) | 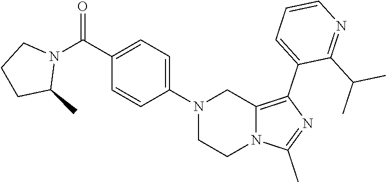 [SMILES: Cc1nc(-c2cccnc2C(C)C)c2n1CCN(c1ccc(C(=O)N3CCC[C@@H]3C)cc1)C2] | 444.5 | |
| | | 3-(3-methyl-7-{4-[(2S)-2-methylpyrrolidine-1- | | |
| | | carbonyl]phenyl}-5H,6H,7H,8H-imidazo[1,5- | | |
| | | a]pyrazin-1-yl)-2-(propan-2-yl)pyridine | | |
| 56 | (+++) | 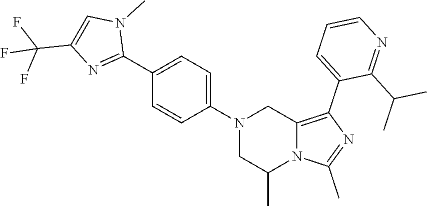 [SMILES: Cc1nc(-c2cccnc2C(C)C)c2n1C(C)CN(c1ccc(-c3nc(C(F)(F)F)cn3C)cc1)C2] | 495.5 | |
| | | 3-(3,5-dimethyl-7-{4-[1-methyl-4- | | |
| | | (trifluoromethyl)-1H-imidazol-2-yl]phenyl}- | | |
| | | 5H,6H,7H,8H-imidazo[1,5-a]pyrazin-1-yl)-2- | | |
| | | (propan-2-yl)pyridine | | |
| 57 | (−) | 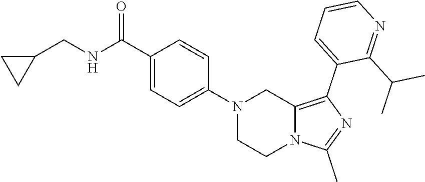 [SMILES: Cc1nc(-c2cccnc2C(C)C)c2n1CCN(c1ccc(C(=O)NCC3CC3)cc1)C2] | 430.5 | |
| | | N-(cyclopropylmethyl)-4-{3-methyl-1-[2- | | |
| | | (propan-2-yl)pyridin-3-yl]-5H,6H,7H,8H- | | |
| | | imidazo[1,5-a]pyrazin-7-yl}benzamide | | |
| 58 | (++) | 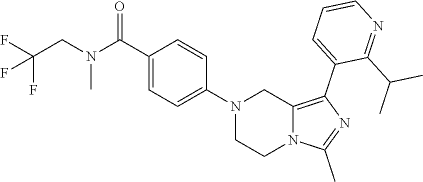 [SMILES: Cc1nc(-c2cccnc2C(C)C)c2n1CCN(c1ccc(C(=O)N(C)CC(F)(F)F)cc1)C2] | 472.5 | |
| | | N-methyl-4-{3-methyl-1-[2-(propan-2-yl)pyridin- | | |
| | | 3-yl]-5H,6H,7H,8H-imidazo[1,5-a]pyrazin-7-yl}- | | |
| | | N-(2,2,2-trifluoroethyl)benzamide | | |
| 59 | (++) | 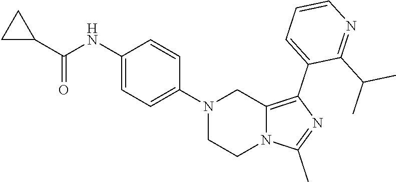 [SMILES: Cc1nc(-c2cccnc2C(C)C)c2n1CCN(c1ccc(NC(=O)C3CC3)cc1)C2] | 416.5 | |
| | | N-(4-{3-methyl-1-[2-(propan-2-yl)pyridin-3-yl]- | | |
| | | 5H,6H,7H,8H-imidazo[1,5-a]pyrazin-7- | | |
| | | yl}phenyl)cyclopropanecarboxamide | | |
| 60 | (−) | 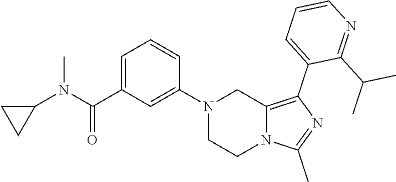 [SMILES: Cc1nc(-c2cccnc2C(C)C)c2n1CCN(c1cccc(C(=O)N(C)C3CC3)c1)C2] | 430.5 | |
| | | N-cyclopropyl-N-methyl-3-{3-methyl-1-[2- | | |
| | | (propan-2-yl)pyridin-3-yl]-5H,6H,7H,8H- | | |
| | | imidazo[1,5-a]pyrazin-7-yl}benzamide | | |
| 61 | (−) | 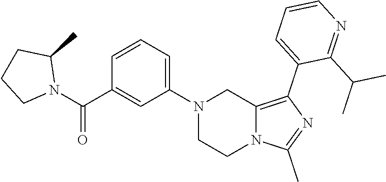 [SMILES: Cc1nc(-c2cccnc2C(C)C)c2n1CCN(c1cccc(C(=O)N3CCC[C@H]3C)c1)C2] | 444.5 | |
| | | 3-(3-methyl-7-{3-[(2R)-2-methylpyrrolidine-1- | | |
| | | carbonyl]phenyl}-5H,6H,7H,8H-imidazo[1,5- | | |
| | | a]pyrazin-1-yl)-2-(propan-2-yl)pyridine | | |
| 62 | (−) | 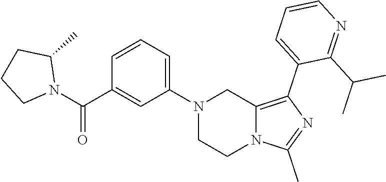 [SMILES: Cc1nc(-c2cccnc2C(C)C)c2n1CCN(c1cccc(C(=O)N3CCC[C@@H]3C)c1)C2] | 444.6 | |
| | | 3-(3-methyl-7-{3-[(2S)-2-methylpyrrolidine-1- | | |
| | | carbonyl]phenyl}-5H,6H,7H,8H-imidazo[1,5- | | |
| | | a]pyrazin-1-yl)-2-(propan-2-yl)pyridine | | |
| 63 | (+++) | 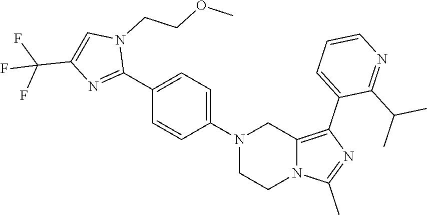 [SMILES: COCCn1cc(C(F)(F)F)nc1-c1ccc(N2CCn3c(C)nc(-c4cccnc4C(C)C)c3C2)cc1] | 525 | (CD₃OD, 300 MHz) δ (ppm): 8.52-8.51 (m, 1H), 7.77-7.64 (m, 2H), 7.52-7.47 (m, 2H), 7.35-7.27 (m, 1H), 7.09-7.06 (m, 2H), 4.42 (s, 2H), 4.22-4.10 (m, 4H), 3.95- 3.87 (m, 2H), 3.69-3.61 (m, 2H), 3.40-3.29 (m, 4H), 2.43 (s, 3H), 1.18 (d, J = 6.4 Hz, 6H). |
| | | 3-(7-{4-[1-(2-methoxyethyl)-4-(trifluoromethyl)- | | |
| | | 1H-imidazol-2-yl]phenyl}-3-methyl- | | |
| | | 5H,6H,7H,8H-imidazo[1,5-a]pyrazin-1-yl)-2- | | |
| | | (propan-2-yl)pyridine | | |
| 64 | (+) | 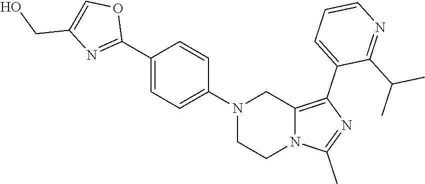 [SMILES: Cc1nc(-c2cccnc2C(C)C)c2n1CCN(c1ccc(-c3nc(CO)co3)cc1)C2] | 430.5 | |
| | | [2-(4-{3-methyl-1-[2-(propan-2-yl)pyridin-3-yl]- | | |
| | | 5H,6H,7H,8H-imidazo[1,5-a]pyrazin-7- | | |
| | | yl}phenyl)-1,3-oxazol-4-yl]methanol | | |
| 65 | (+++) | 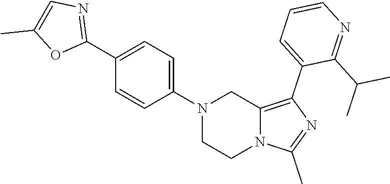 [SMILES: Cc1cnc(-c2ccc(N3CCn4c(C)nc(-c5cccnc5C(C)C)c4C3)cc2)o1] | 414.5 | |
| | | 3-{3-methyl-7-[4-(5-methyl-1,3-oxazol-2- | | |
| | | yl)phenyl]-5H,6H,7H,8H-imidazo[1,5-a]pyrazin- | | |
| | | 1-yl}-2-(propan-2-yl)pyridine | | |
| 66 | (+) | 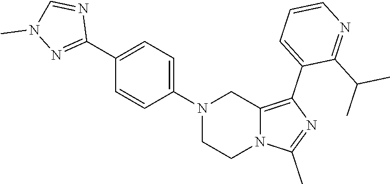 [SMILES: Cc1nc(-c2cccnc2C(C)C)c2n1CCN(c1ccc(-c3ncn(C)n3)cc1)C2] | 414.5 | |
| | | 3-{3-methyl-7-[4-(1-methyl-1H-1,2,4-triazol-3- | | |
| | | yl)phenyl]-5H,6H,7H,8H-imidazo[1,5-a]pyrazin- | | |
| | | 1-yl}-2-(propan-2-yl)pyridine | | |
| 67 | (++) | 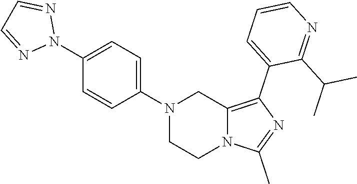 [SMILES: Cc1nc(-c2cccnc2C(C)C)c2n1CCN(c1ccc(-n3nccn3)cc1)C2] | 400.5 | |
| | | 3-{3-methyl-7-[4-(2H-1,2,3-triazol-2-yl)phenyl]- | | |
| | | 5H,6H,7H,8H-imidazo[1,5-a]pyrazin-1-yl}-2- | | |
| | | (propan-2-yl)pyridine | | |
| 68 | (+++) | 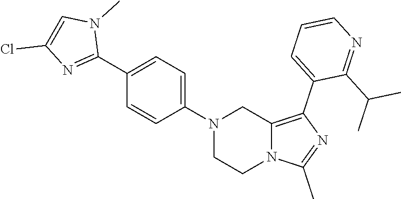 [SMILES: Cc1nc(-c2cccnc2C(C)C)c2n1CCN(c1ccc(-c3nc(Cl)cn3C)cc1)C2] | 447, 449 | (DMSO-d₆, 400 MHz) δ (ppm): 8.49-8.48 (m, 1H), 7.58-7.44 (m, 3H), 7.28-7.17 (m, 2H), 7.07-7.00 (m, 2H), 4.43 (s, 2H), 4.07-4.04 (m, 2H), 3.86-3.84 (m, 2H), 3.65- 3.58 (m, 4H), 2.32 (s, 3H), 1.14 (d, J = 6.8 Hz, 6H). |
| | | 3-{7-[4-(4-chloro-1-methyl-1H-imidazol-2- | | |
| | | yl)phenyl]-3-methyl-5H,6H,7H,8H-imidazo[1,5- | | |
| | | a]pyrazin-1-yl}-2-(propan-2-yl)pyridine | | |
| 69 | (++) | 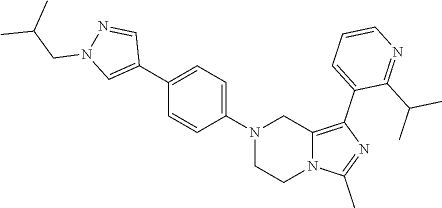 [SMILES: Cc1nc(-c2cccnc2C(C)C)c2n1CCN(c1ccc(-c3cnn(CC(C)C)c3)cc1)C2] | 455.5 | |
| | | 3-(3-methyl-7-{4-[1-(2-methylpropyl)-1H- | | |
| | | pyrazol-4-yl]phenyl}-5H,6H,7H,8H-imidazo[1,5- | | |
| | | a]pyrazin-1-yl)-2-(propan-2-yl)pyridine | | |
| 70 | (+) | 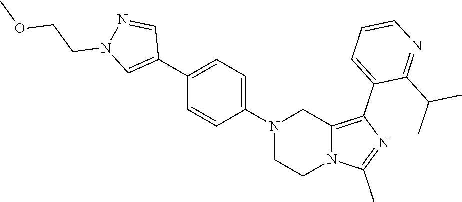 [SMILES: COCCn1cc(-c2ccc(N3CCn4c(C)nc(-c5cccnc5C(C)C)c4C3)cc2)cn1] | 457.5 | |
| | | 3-(7-{4-[1-(2-methoxyethyl)-1H-pyrazol-4- | | |
| | | yl]phenyl}-3-methyl-5H,6H,7H,8H-imidazo[1,5- | | |
| | | a]pyrazin-1-yl)-2-(propan-2-yl)pyridine | | |
| 71 | (−) | 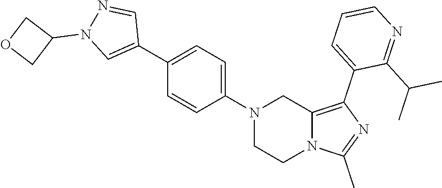 [SMILES: Cc1nc(-c2cccnc2C(C)C)c2n1CCN(c1ccc(-c3cnn(C4COC4)c3)cc1)C2] | 455.5 | |
| | | 3-(3-methyl-7-{4-[1-(oxetan-3-yl)-1H-pyrazol-4- | | |
| | | yl]phenyl}-5H,6H,7H,8H-imidazo[1,5-a]pyrazin- | | |
| | | 1-yl)-2-(propan-2-yl)pyridine | | |
| 72-1 1^{st} eluting isomer 72-2 2^{nd} eluting isomer | (+++) (+++) | 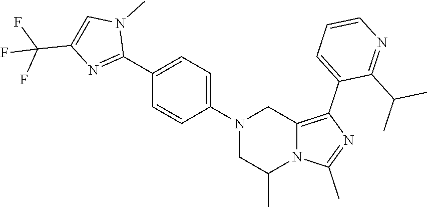 [SMILES: Cc1nc(-c2cccnc2C(C)C)c2n1C(C)CN(c1ccc(-c3nc(C(F)(F)F)cn3C)cc1)C2] | 495 | (CD₃OD, 300 MHz) δ (ppm): 8.38-8.51(m, 1H), 7.78-7.70 (m, 1H), 7.68-7.50 (m, 3H), 7.36-7.28 (m, 1H), 7.12-7.00 (m, 2H), 4.72-4.61 (m, 1H), 4.52 (d, J = 15.1 Hz, 1H), 4.31-4.13 (m, 3H), 3.77 (s, 3H), 3.45-3.34 (m, 1H), 2.47 (s, 3H), 1.35-1.16 (m, 9H). |
| | | 3-[-3,5-dimethyl-7-{4-[1-methyl-4- | | |
| | | (trifluoromethyl)-1H-imidazol-2-yl]phenyl}- | | |
| | | 5H,6H,7H,8H-imidazo[1,5-a]pyrazin-1-yl]-2- | | |
| | | (propan-2-yl)pyridine | | |
| 73 | (+++) | 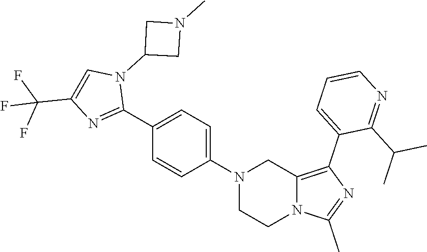 [SMILES: Cc1nc(-c2cccnc2C(C)C)c2n1CCN(c1ccc(-c3nc(C(F)(F)F)cn3C3CN(C)C3)cc1)C2] | 536 | (CD₃OD, 400 MHz) δ (ppm): 8.52-8.50 (m, 1H), 8.06 (s, 1H), 7.68-7.65 (m, 1H), 7.39-7.36 (m, 2H), 7.32- 7.29 (m, 1H), 7.09-7.07 (m, 2H), 4.94-4.91 (m, 1H), 4.40 (s, 2H), 4.15-4.13 (m, 2H), 3.92-3.89 (m, 2H), 3.74-3.70 (m, 2H), 3.41-3.3 (m, 3H), 2.40-2.38 (m, 6H), 1.22-1.19 (m, 6H). |
| | | 3-(3-methyl-7-{4-[1-(1-methylazetidin-3-yl)-4- | | |
| | | (trifluoromethyl)-1H-imidazol-2-yl]phenyl}- | | |
| | | 5H,6H,7H,8H-imidazo[1,5-a]pyrazin-1-yl)-2- | | |
| | | (propan-2-yl)pyridine | | |
| 74 | (+) | 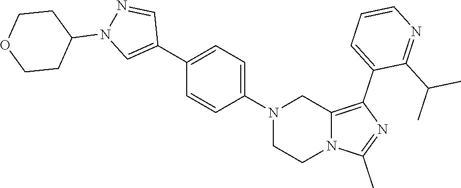 [SMILES: Cc1nc(-c2cccnc2C(C)C)c2n1CCN(c1ccc(-c3cnn(C4CCOCC4)c3)cc1)C2] | 483.6 | |
| | | 3-(3-methyl-7-{4-[1-(oxan-4-yl)-1H-pyrazol-4- | | |
| | | yl]phenyl}-5H,6H,7H,8H-imidazo[1,5-a]pyrazin- | | |
| | | 1-yl)-2-(propan-2-yl)pyridine | | |
| 75 | (+++) | 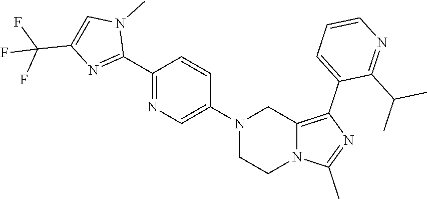 [SMILES: Cc1nc(-c2cccnc2C(C)C)c2n1CCN(c1ccc(-c3nc(C(F)(F)F)cn3C)nc1)C2] | 482.5 | |
| | | 5-{3-methyl-1-[2-(propan-2-yl)pyridin-3-yl]- | | |
| | | 5H,6H,7H,8H-imidazo[1,5-a]pyrazin-7-yl}-2-[1- | | |
| | | methyl-4-(trifluoromethyl)-1H-imidazol-2- | | |
| | | yl]pyridine | | |
| 76 | (+++) | 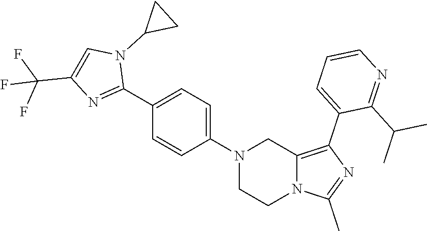 [SMILES: Cc1nc(-c2cccnc2C(C)C)c2n1CCN(c1ccc(-c3nc(C(F)(F)F)cn3C3CC3)cc1)C2] | 507 | (CD₃OD, 400 MHz) δ (ppm): 8.52-8.50 (m, 1H), 7.70-7.64 (m, 4H), 7.32-7.29 (m, 1H), 7.09-7.07 (m, 2H), 4.42-4.39 (m, 2H), 4.16-4.13 (m, 2H), 3.92-3.89 (m, 2H), 3.60-3.57(m, 1H), 3.38-3.31 (m, 1H), 2.41 (s, 3H), 1.22- 1.19 (m, 6H), 1.02-0.98 (m, 2H), 0.90-0.83 (m, 2H). |
| | | 3-(7-{4-[1-cyclopropyl-4-(trifluoromethyl)-1H- | | |
| | | imidazol-2-yl]phenyl}-3-methyl-5H,6H,7H,8H- | | |
| | | imidazo[1,5-a]pyrazin-1-yl)-2-(propan-2- | | |
| | | yl)pyridine | | |
| 77 | (+++) | 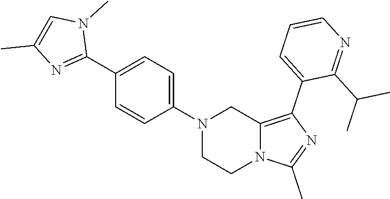 [SMILES: Cc1cn(C)c(-c2ccc(N3CCn4c(C)nc(-c5cccnc5C(C)C)c4C3)cc2)n1] | 427 | (CD₃OD, 400 MHz) δ (ppm): 8.55-8.47(m, 1H), 7.71-7.62 (m, 1H), 7.53-7.44 (m, 2H), 7.35-7.25 (m, 1H), 7.13-7.04 (m, 2H), 6.79-6.77 (m, 1H), 4.39 (s, 2H), 4.14- 4.11 (m, 2H), 3.91-3.82 (m, 2H), 3.62-3.60 (m, 3H), 3.45- 3.34 (m, 1H), 2.43 (s, 3H), 2.16 (s, 3H), 1.22-1.17 (m, 6H). |
| | | 3-{7-[4-(1,4-dimethyl-1H-imidazol-2-yl)phenyl]- | | |
| | | 3-methyl-5H,6H,7H,8H-imidazo[1,5-a]pyrazin-1- | | |
| | | yl}-2-(propan-2-yl)pyridine | | |
| 78 | (+++) | 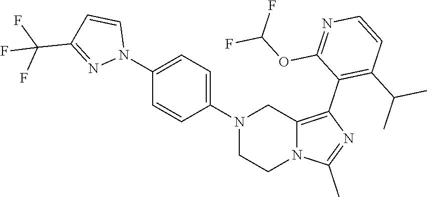 [SMILES: Cc1nc(-c2c(C(C)C)ccnc2OC(F)F)c2n1CCN(c1ccc(-n3ccc(C(F)(F)F)n3)cc1)C2] | 491.4 | |
| | | 2-(difluoromethoxy)-3-(3-methyl-7-{4-[3- | | |
| | | (trifluoromethyl)-1H-pyrazol-1-yl]phenyl}- | | |
| | | 5H,6H,7H,8H-imidazo[1,5-a]pyrazin-1- | | |
| | | yl)pyridine | | |
| 79 | (+++) | 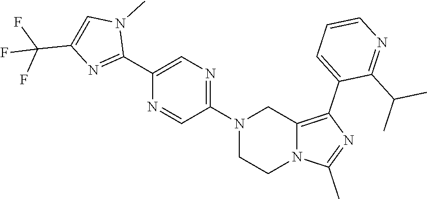 [SMILES: Cc1nc(-c2cccnc2C(C)C)c2n1CCN(c1cnc(-c3nc(C(F)(F)F)cn3C)cn1)C2] | 483.5 | |
| | | 2-{3-methyl-1-[2-(propan-2-yl)pyridin-3-yl]- | | |
| | | 5H,6H,7H,8H-imidazo[1,5-a]pyrazin-7-yl}-5-[1- | | |
| | | methyl-4-(trifluoromethyl)-1H-imidazol-2- | | |
| | | yl]pyrazine | | |
| 80 | (+++) | 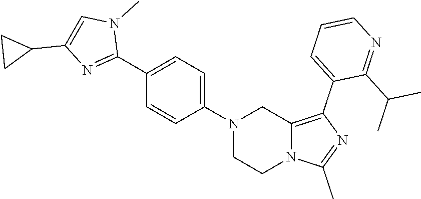 [SMILES: Cc1nc(-c2cccnc2C(C)C)c2n1CCN(c1ccc(-c3nc(C4CC4)cn3C)cc1)C2] | 453 | (CD₃OD, 400 MHz) δ (ppm): 8.55-8.48(m, 1H), 7.70-7.62(m, 1H), 7.51-7.43 (m, 2H), 7.34-7.25 (m, 1H), 7.13-7.04 (m, 2H), 6.78 (s, 1H), 4.39 (s, 2H), 4.18-4.10 (m, 2H), 3.91-3.84 (m, 2H), 3.62 (s, 3H), 3.44-3.34 (m, 1H), 2.43 (s, 3H), 1.85-1.74 (m, 1H), 1.24 (d, J = 6.8 Hz, 6H), 0.88-0.77 (m, 2H), 0.72- 0.61 (m, 2H). |
| | | 3-{7-[4-(4-cyclopropyl-1-methyl-1H-imidazol-2- | | |
| | | yl)phenyl]-3-methyl-5H,6H,7H,8H-imidazo[1,5- | | |
| | | a]pyrazin-1-yl}-2-(propan-2-yl)pyridine | | |
| 81 | (+++) | 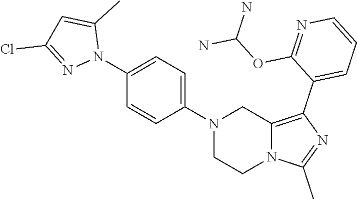 [SMILES: Cc1cc(Cl)nn1-c1ccc(N2CCn3c(C)nc(-c4cccnc4OC(N)N)c3C2)cc1] | 471, 473 | (CD₃OD, 400 MHz) δ (ppm): 8.18-8.16 (m, 1H), 8.04-8.02 (m, 1H), 7.78 (t, J = 72.8 Hz, 1H), 7.34-7.25 (m, 3H), 7.19-7.06 (m, 2H), 6.18 (s, 1H), 4.52 (s, 2H), 4.09 (t, J = 5.6 Hz, 2H), 3.84 (t, J = 5.6 Hz, 2H), 2.41 (s, 3H), 2.24 (s, 3H). |
| | | 3-{7-[4-(3-chloro-5-methyl-1H-pyrazol-1- | | |
| | | yl)phenyl]-3-methyl-5H,6H,7H,8H-imidazo[1,5- | | |
| | | a]pyrazin-1-yl}-2-(difluoromethoxy)pyridine | | |
| 82 | (+++) | 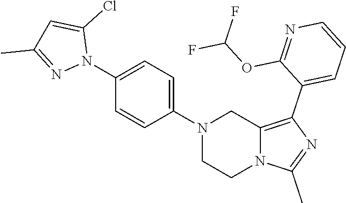 [SMILES: Cc1cc(Cl)n(-c2ccc(N3CCn4c(C)nc(-c5cccnc5OC(F)F)c4C3)cc2)n1] | 471, 473 | (CD₃OD, 400 MHz) δ (ppm): 8.09-8.07 (m, 1H), 7.93-7.90 (m, 1H), 7.66 (t, J = 73.2 Hz, 1H), 7.27-7.16 (m, 3H), 7.04-7.01 (m, 2H), 6.19-6.17 (m, 1H), 4.44 (s, 2H), 4.14 (t, J = 5.6 Hz, 2H), 3.76 (t, J = 5.6 Hz, 2H), 2.32 (s, 3H), 2.15 (s, 3H). |
| | | 3-{7-[4-(5-chloro-3-methyl-1H-pyrazol-1- | | |
| | | yl)phenyl]-3-methyl-5H,6H,7H,8H-imidazo[1,5- | | |
| | | a]pyrazin-1-yl}-2-(difluoromethoxy)pyridine | | |
| 83 | (−) | 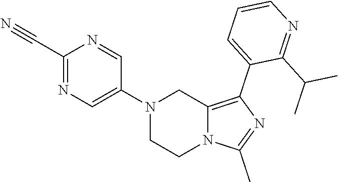 [SMILES: Cc1nc(-c2cccnc2C(C)C)c2n1CCN(c1cnc(C#N)nc1)C2] | 360.4 | |
| | | 5-{3-methyl-1-[2-(propan-2-yl)pyridin-3-yl]- | | |
| | | 5H,6H,7H,8H-imidazo[1,5-a]pyrazin-7- | | |
| | | yl}pyrimidine-2-carbonitrile | | |
| 84 | (+++) | 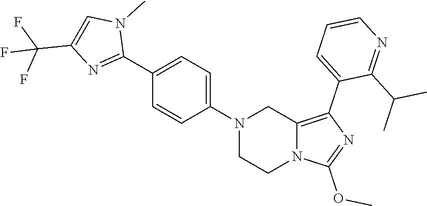 [SMILES: COc1nc(-c2cccnc2C(C)C)c2n1CCN(c1ccc(-c3nc(C(F)(F)F)cn3C)cc1)C2] | 497 | (CD₃OD, 400 MHz) δ (ppm): 8.51-8.49 (m, 1H), 7.70-7.68 (m, 1H), 7.61 (d, J = 0.8 Hz, 1H), 7.52-7.49 (m, 2H), 7.31-7.28 (m, 1H), 7.08- 7.06 (m, 2H), 4.37 (s, 2H), 4.04 (s, 3H), 3.99-3.92 (m, 2H), 3.90-3.85 (m, 2H), 3.76 (s, 3H), 3.52-3.44 (m, 1H), 1.24 (d, J = 6.8 Hz, 6H). |
| | | 3-(3-methoxy-7-{4-[1-methyl-4- | | |
| | | (trifluoromethyl)-1H-imidazol-2-yl]phenyl}- | | |
| | | 5H,6H,7H,8H-imidazo[1,5-a]pyrazin-1-yl)-2- | | |
| | | (propan-2-yl)pyridine | | |
| 85 | (+++) | 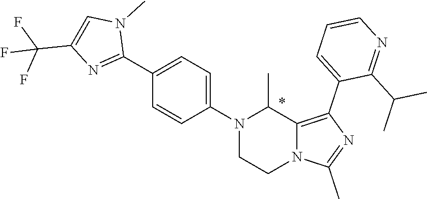 [SMILES: Cc1nc(-c2cccnc2C(C)C)c2n1CCN(c1ccc(-c3nc(C(F)(F)F)cn3C)cc1)C2C] | 495 | (CD₃OD, 300 MHz) δ (ppm): 8.55-8.53 (m, 1H), 7.72-7.69 (m, 1H), 7.63 (s, 1H), 7.55 (d, J = 9.0 Hz, 2H), 7.34-7.30 (m, 1H), 7.10 (d, J = 9.0 Hz, 2H), 5.19-5.12 (m, 1H), 4.20-4.03 (m, 3H), 3.77- 3.68 (m, 4H), 3.27-3.22 (m, 1H), 2.42 (s, 3H), 1.31 (d, J = 6.9 Hz, 3H), 1.23 (d, J = 6.6 Hz, 3H), 1.13 (d, J = 6.9 Hz, 3H). |
| | | 3-[3,8-dimethyl-7-{4-[1-methyl-4- | | |
| | | (trifluoromethyl)-1H-imidazol-2-yl]phenyl}- | | |
| | | 5H,6H,7H,8H-imidazo[1,5-a]pyrazin-1-yl]-2- | | |
| | | (propan-2-yl)pyridine | | |
| 86 | (+++) | 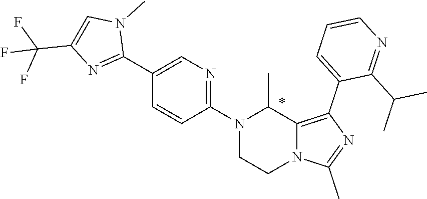 [SMILES: Cc1nc(-c2cccnc2C(C)C)c2n1CCN(c1ccc(-c3nc(C(F)(F)F)cn3C)cn1)C2C] | 496 | (CD₃OD, 300 MHz) δ (ppm): 8.60-8.59 (m, 1H), 8.43-8.42 (m, 1H), 7.87-7.83 (m, 1H), 7.76-7.73 (m, 1H), 7.67-7.66 (m, 1H), 7.38-7.33 (m, 1H), 7.02 (d, J = 9.0 Hz, 1H), 5.85-5.83 (m, 1H), 4.69- 4.64 (m, 1H), 4.28-4.23 (m, 1H), 4.12-4.03 (m, 1H), 3.77- 3.74 (m, 4H), 3.19-3.16 (m, 1H), 2.48 (s, 3H), 1.33 (d, J = 6.9 Hz, 3H), 1.28 (d, J = 6.6 Hz, 3H), 1.14 (d, J = 6.9 Hz, 3H). |
| | | 2-[3,8-dimethyl-1-[2-(propan-2-yl)pyridin-3-yl]- | | |
| | | 5H,6H,7H,8H-imidazo[1,5-a]pyrazin-7-yl]-5-[1- | | |
| | | methyl-4-(trifluoromethyl)-1H-imidazol-2- | | |
| | | yl]pyridine | | |

*Absolute stereochemistry not determined.

TABLE 6
Biological Data of Additional Compounds.

| Cmpd. No. | IC₅₀ (μM) | Name | Structure |
|---|---|---|---|
| C-1 | (++) | 1-(2-isopropylpyridin-3-yl)-3- methyl-7-(4-(1-methyl-4- (trifluoromethyl)-1H-imidazol-2- yl)phenyl)-6,7,8,9-tetrahydro-5H- imidazo[1,5-d][1,4]diazepine | 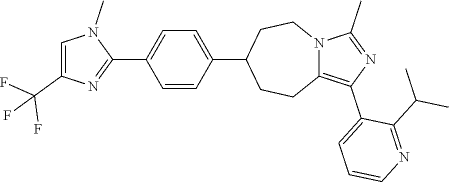 [SMILES: Cc1nc(-c2cccnc2C(C)C)c2n1CCC(c1ccc(-c3nc(C(F)(F)F)cn3C)cc1)CC2] |
| C-2 | (++) | 1-(2-isopropylpyridin-3-yl)-3- methyl-8-(4-(1-methyl-4- (trifluoromethyl)-1H-imidazol-2- yl)benzyl)-6,7,8,9-tetrahydro-5H- imidazo[1,5-a][1,4]diazepine | 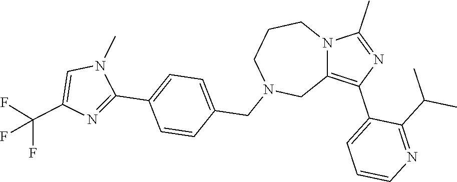 [SMILES: Cc1nc(-c2cccnc2C(C)C)c2n1CCCN(Cc1ccc(-c3nc(C(F)(F)F)cn3C)cc1)C2] |
| C-3 | (−) | 1-(2-isopropylpyridin-3-yl)-3- methyl-7-(4-(1-methyl-4- (trifluoromethyl)-1H-imidazol-2- yl)phenyl)-7,8-dihydroimidazo[1,5- a]pyrazin-6(5H)-one | 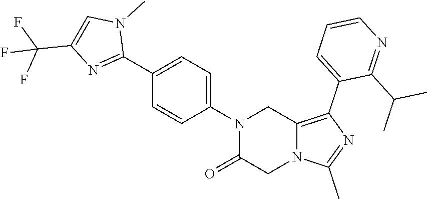 [SMILES: Cc1nc(-c2cccnc2C(C)C)c2n1CC(=O)N(c1ccc(-c3nc(C(F)(F)F)cn3C)cc1)C2] |
| C-4 | (−) | 3-(2-isopropylphenyl)-1-methyl-6-(4- (1-methyl-4-(trifluoromethyl)-1H- imidazol-2-yl)piperidin-1- yl)imidazo[1,5-a]pyridine | 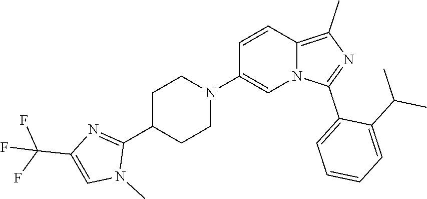 [SMILES: Cc1nc(-c2ccccc2C(C)C)n2cc(N3CCC(c4nc(C(F)(F)F)cn4C)CC3)ccc12] |
| C-5 | (−) | 1-(2-isopropylpyridin-3-yl)-3- methyl-7-(4-(1-methyl-4- (trifluoromethyl)-1H-imidazol-2- yl)phenyl)-6,7-dihydroimidazo[1,5- a]pyrazin-8(5H)-one | 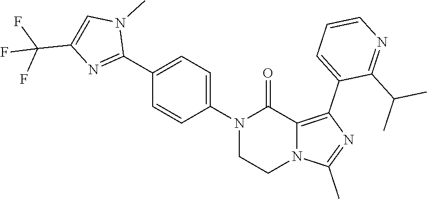 [SMILES: Cc1nc(-c2cccnc2C(C)C)c2n1CCN(c1ccc(-c3nc(C(F)(F)F)cn3C)cc1)C2=O] |
| C-6 | (−) | 3-(3-methyl-7-{6-[1-(propan-2-yl)- 1H-pyrazol-5-yl]pyridine-3- carbonyl}-5H,6H,7H,8H- imidazo[1,5-a]pyrazin-1-yl)-2- (propan-2-yl)pyridine | 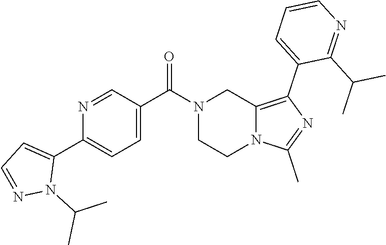 [SMILES: Cc1nc(-c2cccnc2C(C)C)c2n1CCN(C(=O)c1ccc(-c3ccnn3C(C)C)nc1)C2] |

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

We claim:

1. A compound of Formula I: 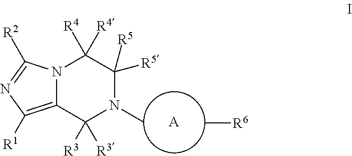
or a pharmaceutically acceptable salt thereof, wherein:
Ring A is —C₃-C₁₂ cycloalkyl, 3- to 14-membered heterocyclyl having 1 to 4 heteroatoms selected from oxygen, nitrogen, and sulfur, —C₆-C₁₀ aryl, or 5- to 14-membered heteroaryl having 1 to 4 heteroatoms selected from oxygen, nitrogen, and sulfur,
wherein each cycloalkyl, heterocyclyl, aryl, or heteroaryl of Ring A is optionally substituted with one or more substituents selected from the group consisting of halogen, —C₁-C₆ alkyl, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, and —S(O)₂NR₂;
R¹ is —C₆-C₁₀ aryl or 5- to 14-membered heteroaryl having 1 to 4 heteroatoms selected from oxygen, nitrogen, and sulfur,
wherein each aryl or heteoraryl of R¹ is optionally substituted with one or more R^{1a};
each R^{1a} is independently halogen, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, —C₁-C₆ alkyl, —C₂-C₆ alkenyl, —C₂-C₆ alkynyl, —C₃-C₁₂ cycloalkyl, —C₄-C₁₂ cycloalkenyl, 3- to 14-membered heterocyclyl having 1 to 4 heteroatoms selected from oxygen, nitrogen, or sulfur, —C₆-C₁₀ aryl, or 5- to 14-membered heteroaryl having 1 to 4 heteroatoms selected from oxygen, nitrogen, or sulfur;
R² is —H, halogen, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, —C₁-C₆ alkyl, —C₂-C₆ alkenyl, —C₂-C₆ alkynyl, —C₃-C₁₂ cycloalkyl, —C₄-C₁₂ cycloalkenyl, 3- to 14-membered heterocyclyl having 1 to 4 heteroatoms selected from oxygen, nitrogen, or sulfur, —C₆-C₁₀ aryl, or 5- to 14-membered heteroaryl having 1 to 4 heteroatoms selected from oxygen, nitrogen, or sulfur,
wherein each alkyl, alkenyl, alkynyl, cycloalkyl, cycloalkenyl, heterocyclyl, aryl, or heteroaryl of R² is optionally substituted with one or more halogen, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, —C₁-C₆ alkyl, —C₃-C₁₂ cycloalkyl, 3- to 14-membered heterocyclyl having 1 to 4 heteroatoms selected from oxygen, nitrogen, or sulfur, —C₆-C₁₀ aryl, or 5- to 14-membered heteroaryl having 1 to 4 heteroatoms selected from oxygen, nitrogen, or sulfur;
R³, R^{3′}, R⁴, R^{4′}, R⁵, and R^{5′} are each independently selected from the group consisting of —H, —C₁-C₆ alkyl, —C₂-C₆ alkenyl, —C₂-C₆ alkynyl, and —C₃-C₇ cycloalkyl;
or each of (R³ and R^{3′}), or (R⁴ and R^{4′}), or (R⁵ and R^{5′}) can combine with the atom to which they are attached to form a —C₃-C₁₂ cycloalkyl ring or 3- to 14-membered heterocyclyl ring having 1 to 4 heteroatoms selected from oxygen, nitrogen, or sulfur;
R⁶ is —H, halogen, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, —C₁-C₆ alkyl, —C₂-C₆ alkenyl, —C₂-C₆ alkynyl, —C₃-C₁₂ cycloalkyl, —C₄-C₁₂ cycloalkenyl, 3- to 14-membered heterocyclyl having 1 to 4 heteroatoms selected from oxygen, nitrogen, or sulfur, —C₆-C₁₀ aryl, or 5- to 14-membered heteroaryl having 1 to 4 heteroatoms selected from oxygen, nitrogen, or sulfur,
wherein each alkyl, alkenyl, alkynyl, cycloalkyl, cycloalkenyl, heterocyclyl, aryl or heteroaryl of R⁶ is optionally substituted with one or more R^{6a};
each R^{6a} is independently selected from the group consisting of halogen, oxo, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, —S(O)₂NR₂, —C₁-C₆ alkyl, —C₂-C₆ alkenyl, —C₂-C₆ alkynyl, —C₃-C₁₂ cycloalkyl, —C₄-C₁₂ cycloalkenyl, 3- to 14-membered heterocyclyl having 1 to 4 heteroatoms selected from oxygen, nitrogen, or sulfur, —C₆-C₁₀ aryl, and 5- to 14-membered heteroaryl having 1 to 4 heteroatoms selected from oxygen, nitrogen, or sulfur,
wherein each alkyl, alkenyl, alkynyl, cycloalkyl, cycloalkenyl, heterocyclyl, aryl, or heteroaryl of R^{6a} is optionally substituted with one or more halogen, —C₁-C₆ alkyl, or —OR;
each R is independently selected from the group consisting of —H, —C₁-C₆ alkyl, —C₀-C₆ alkylene-C₆-C₁₀ aryl, —C₃-C₁₂ cycloalkyl, 3- to 14-membered heterocyclyl having 1 to 4 heteroatoms selected from oxygen, nitrogen, or sulfur, or 5- to 14-membered heteroaryl having 1 to 4 heteroatoms selected from oxygen, nitrogen, or sulfur,
wherein each alkyl, aryl, cycloalkyl, heterocyclyl, or heteroaryl of R is optionally substituted with one or more R^{a};
or two R groups can combine with the atom to which they are attached to form a 5- to 6-membered heterocyclyl having 1 to 4 heteroatoms selected from oxygen, nitrogen, or sulfur, optionally substituted with —C₁-C₆ alkyl;
each R^{a} is independently halogen, —O(C₁-C₆ alkyl), —NH(C₁-C₆ alkyl), —N(C₁-C₆ alkyl)₂, —C₃-C₆ cycloalkyl, 5- to 6-membered heterocyclyl having 1 to 4 heteroatoms selected from oxygen, nitrogen, or sulfur, or 5- to 6-membered heteroaryl having 1 to 4 heteroatoms selected from oxygen, nitrogen, or sulfur,
wherein each cycloalkyl, heterocyclyl, or heteroaryl of R^{a} is optionally substituted with —C₁-C₆ alkyl or —C₁-C₆ alkyl substituted with one or more halogen; and
each R′ is independently selected from the group consisting of —C₁-C₆ alkyl, —C₂-C₆ alkenyl, —C₂-C₆ alkynyl, —C₃-C₁₂ cycloalkyl, —C₄-C₁₂ cycloalkenyl, 3- to 14-membered heterocyclyl having 1 to 4 heteroatoms selected from oxygen, nitrogen, or sulfur, —C₆-C₁₀ aryl, and 5- to 14-membered heteroaryl having 1 to 4 heteroatoms selected from oxygen, nitrogen, or sulfur.

2. The compound of claim 1, wherein Ring A is phenyl or 6-membered heteroaryl having 1 to 3 nitrogen atoms, wherein each phenyl or heteroaryl of Ring A is optionally substituted with one or more substituents selected from the group consisting of halogen, —C₁-C₆ alkyl, —OR, —OC(O)R′, —NR₂, —NRC(O)R′, —NRS(O)₂R′, —CN, —NO₂, —SR, —C(O)R′, —C(O)OR, —C(O)NR₂, —S(O)₂R′, and —S(O)₂NR₂.

3. The compound of claim 1 or claim 2, wherein R¹ is phenyl or 6-membered heteroaryl having 1 to 3 nitrogen atoms, wherein each phenyl or heteroaryl is substituted with 0, 1, 2, 3, or 4 R^{1a}.

4. The compound of any one of claims 1-3, wherein R^{1a} is —OR or —C₁-C₆ alkyl.

5. The compound of any one of claims 1-4, wherein R^{1a} is isopropyl or —OCHF₂.

6. The compound of any one of claims 1-5, wherein R² is —H, —OR, —C₁-C₆ alkyl, or —C₃-C₁₂ cycloalkyl, wherein each alkyl or cycloalkyl of R² is optionally substituted with one or more halogen.

7. The compound of any one of claims 1-6, wherein R³, R^{3′}, R⁴, R^{4′}, R⁵, and R^{5′} are each independently selected from the group consisting of —H and —C₁-C₆ alkyl.

8. The compound of any one of claims 1-7, wherein R⁶ is selected from the group consisting of —H, halogen, —OR, —NR₂, —NRC(O)R′, —CN, —C(O)NR₂, —C₁-C₆ alkyl, 3- to 14-membered heterocyclyl having 1 to 4 heteroatoms selected from oxygen, nitrogen, or sulfur, or 5- to 14-membered heteroaryl having 1 to 4 heteroatoms selected from oxygen, nitrogen, or sulfur, wherein each alkyl, heterocyclyl, aryl, or heteroaryl of R⁶ is substituted with 0, 1, 2, 3, or 4 R^{6a}.

9. The compound of any one of claims 1-8, wherein R⁶ is 5-membered heteroaryl having 1 to 4 heteroatoms selected from oxygen, nitrogen, and sulfur, wherein the heteroaryl of R⁶ is substituted with 0, 1, or 2 R^{6a}.

10. The compound of any one of claims 1-9, wherein each R is independently selected from the group consisting of —H and —C₁-C₆ alkyl and wherein each alkyl of R is substituted with 0, 1, 2, 3, or 4 R^{a}.

11. The compound of any one of claims 1-10, wherein the compound is of Formula IIIa or IIIb: 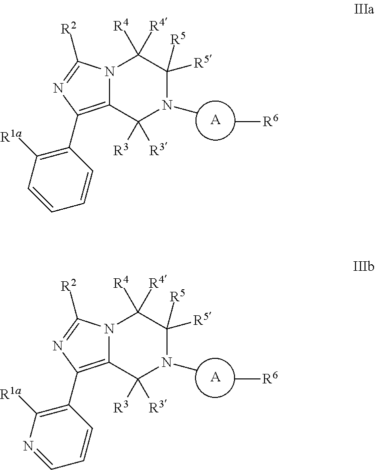
or a pharmaceutically acceptable salt thereof.

12. The compound of any one claims 1-11, wherein the compound is of Formula IVa or Formula IVb: 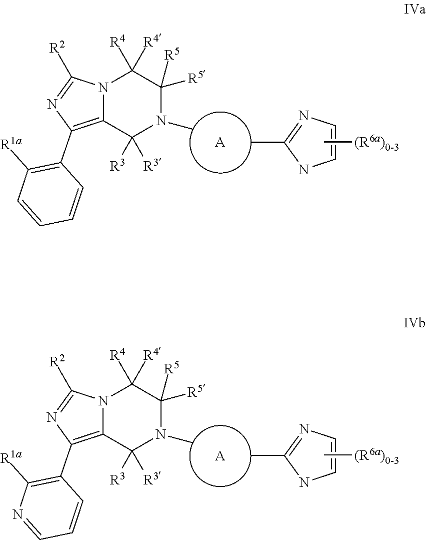
or a pharmaceutically acceptable salt thereof.

13. The compound of any one of the preceding embodiments, wherein the compound is of Formula Va or Formula Vb: 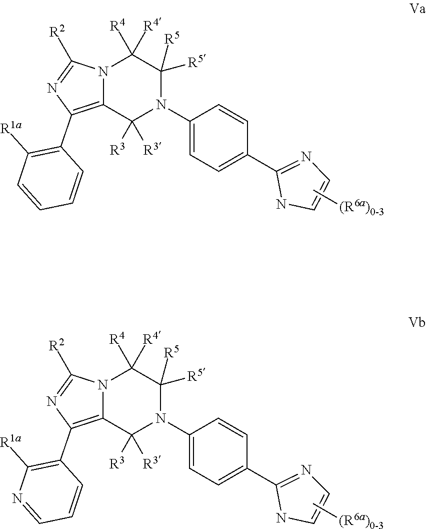
or a pharmaceutically acceptable salt thereof.

14. A compound, or a pharmaceutically acceptable salt thereof, selected from Table 1.

15. The compound of claim 14, wherein the compound is: 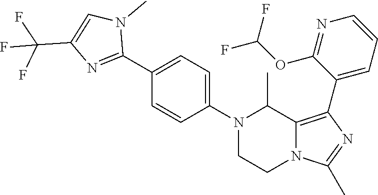 [SMILES: Cc1nc(-c2cccnc2OC(F)F)c2n1CCN(c1ccc(-c3nc(C(F)(F)F)cn3C)cc1)C2C]
or a pharmaceutically acceptable salt thereof.

16. The compound of claim 14, wherein the compound is: 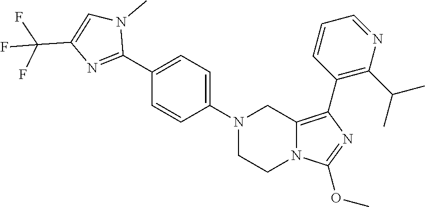 [SMILES: COc1nc(-c2cccnc2C(C)C)c2n1CCN(c1ccc(-c3nc(C(F)(F)F)cn3C)cc1)C2]
or a pharmaceutically acceptable salt thereof.

17. The compound of claim 14, wherein the compound is: 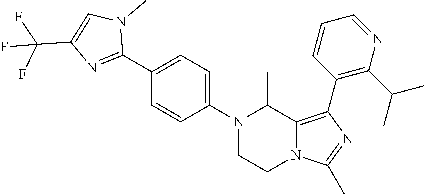 [SMILES: Cc1nc(-c2cccnc2C(C)C)c2n1CCN(c1ccc(-c3nc(C(F)(F)F)cn3C)cc1)C2C]
or a pharmaceutically acceptable salt thereof.

18. The compound of claim 14, wherein the compound is: 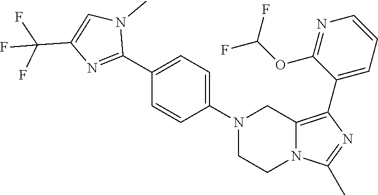 [SMILES: Cc1nc(-c2cccnc2OC(F)F)c2n1CCN(c1ccc(-c3nc(C(F)(F)F)cn3C)cc1)C2]
or a pharmaceutically acceptable salt thereof.

19. A pharmaceutical composition comprising a compound of any one of claims 1-18, or a pharmaceutically acceptable salt thereof, and a pharmaceutically acceptable carrier.
